(19) United States
(12) Patent Application Publication
(10) Pub. No.: US 2024/0335585 A1
(43) Pub. Date: Oct. 10, 2024

(54) TISSUE-ENGINEERED SCAFFOLDS AND METHODS OF MAKING

(71) Applicant: GEORGIA TECH RESEARCH CORPORATION, Atlanta, GA (US)

(72) Inventors: Chen JIANG, Atlanta, GA (US); Kan WANG, Atlanta, GA (US); Ben WANG, Atlanta, GA (US)

(21) Appl. No.: 18/573,671

(22) PCT Filed: Apr. 6, 2022
(86) PCT No.: PCT/US2022/023678

§ 371 Date: Dec. 22, 2023

Related U.S. Application Data
(60) Provisional application No. 63/215,045, filed on Jun. 25, 2021.
(60) Provisional application No. 63/215,049, filed on Jun. 25, 2021.

(51) Int. Cl. A61L 27/18; A61L 27/34; A61L 27/38

(52) U.S. Cl. CPC A61L 27/18; A61L 27/34; A61L 27/38

(57) ABSTRACT

Described are scaffolds for tissue engineering and method of making and using the scaffold. The method of making can include providing a textile layer formed of a plurality of yarns, wherein the plurality of yarns are formed of interlocking bundles of fibers formed from a first polymer or a first polymer mixture; and forming one or more substrate layer of a second polymer or a second polymer mixture onto the textile layer having a pre-defined thickness.

### CROSS-REFERENCE TO RELATED APPLICATIONS

The application claims the benefit of U.S. Provisional Application No. 63/215,045, filed Jun. 25, 2021, and U.S. Provisional Application No. 63/215,049, filed Jun. 25, 2021, which are incorporated herein by reference in their entirety.

### BACKGROUND

The demand for organ repair and transplantation, and a shortage of available donors, necessitate the clinical need to develop innovative strategies for the repair and regeneration of injured or diseased tissues and organs. Tissue engineering, which aims to create living biological substitutes, offers promising avenues to address the organ shortage. One of the key elements in tissue engineering is to design and fabricate scaffolds with tissue-like properties. Failure to mimic the properties of human tissues can have negative results in tissue integration and regeneration.

A challenge in scaffold fabrication is to mimic the mechanical properties of human tissues. While human tissues have strain-stiffening properties, most synthetic biopolymers, e.g., currently employed for tissue engineering applications, have strain-softening properties. There are different tissue types, each having a specific strain-stiffening property.

There is a benefit to engineering new tissues with strain-stiffening properties.

### SUMMARY

Provided herein are methods of making a scaffold for tissue engineering including: providing a textile layer formed of a plurality of yarns, wherein the plurality of yarns are formed of interlocking bundles of fibers formed from a first polymer or a first polymer mixture; and forming one or more substrate layer of a second polymer or a second polymer mixture onto the textile layer having a predefined thickness.

In some embodiments, the method can further include: forming a yarn of the plurality of fibers by electrospinning the first polymer or the first polymer mixture into the plurality of fibers; drawing the plurality of fibers out of an electrospinning bath to form the bundles; and forming the yarn from the bundles. In some embodiments, the step of drawing the plurality of fibers out of the electrospinning bath to form the bundles can further include winding the plurality of drawn fibers around a roller. In some embodiments, the method can further include varying a drawing speed to generate (i) a desired fiber alignment of the plurality of fibers, (ii) a desired bundle diameter of the plurality of fibers, or a combination thereof. In some embodiments, the method can further include seeding a population of cells onto the scaffold.

In some embodiments, forming the textile layer can include crocheting the plurality of yarns with a pre-defined crochet hook size to provide the scaffold with a pre-defined mechanical property selected from a pre-defined resilience or range, a pre-defined elastic modulus or range, a pre-defined maximum strain or range, pre-defined maximum stress or range, or any combination thereof. In some embodiments, forming the textile layer can include coating the plurality of yarns with a biomaterial that promotes cell adhesion.

In some embodiments, the method can further include: electrospinning the one or more substrate layers; and varying the duration of the electrospinning step to achieve a desired substrate layer thickness.

In some embodiments, the electrospinning bath can include a liquid having a concentration in the electrospinning bath to form the plurality of fibers having a pre-defined mechanical property with the one or more substrate layers. In some embodiments, the electrospinning bath can further include a carbon nanomaterial to generate the plurality of fibers with a carbon nanomaterial coating.

Described herein are also composite yarns including: a yarn core comprising one or more polymers and carbon nanomaterial on the surface of the yarn core. In some embodiments, the composite yarns can be used to form a textile layer of a scaffold for tissue engineering. In some embodiments, the composite yarns can be formed of interlocking bundles of fibers, including the one or more polymers and the carbon nanomaterial.

Described herein are also methods of fabricating composite yarns, the method can include: forming fibers of one or more polymers in a carbon nanomaterial bath, the carbon nanomaterial bath can include a carbon nanomaterial suspended in a liquid; coating the fibers with the carbon nanomaterial to form fibers; extracting the fibers from the carbon nanomaterial bath; and interlocking bundles of fibers to form composite yarns, the composite yarns can include the one or more polymers and carbon nanomaterial. In some embodiments, the method can further include coating the composite yarns with a biomaterial that promotes cell adhesion.

Described herein are also methods of fabricating a scaffold for tissue engineering, the method can include: fabricating composite yarns according to the methods described herein; and forming a scaffold including the composite yarns described herein. In some embodiments, forming the scaffold can include coating the composite yarns with a biomaterial that promotes cell adhesion.

Provided herein are also scaffolds for tissue engineering formed by the methods described herein. In some embodiments, the scaffold can include: a textile layer formed of a plurality of yarns, wherein the plurality of yarns can be formed of interlocking bundles of fibers formed from a first polymer or a first polymer mixture; and one or more substrate layers, including a second polymer or a second polymer mixture formed onto or attached to the textile layer. In some embodiments, the method can further include a second textile layer. In some embodiments, the second textile layer can be attached to (i) the textile layer, (ii) the one or more substrate layers of the textile layer, or any combination thereof. In some embodiments, the scaffolds for tissue engineering can include the composite yarns described herein.

Described herein are also therapeutic methods can include: providing the scaffold described herein; and implanting the scaffold into or onto a subject.

Described herein are also methods of promoting cell adhesion to a tissue-engineered scaffold, the method can include: fabricating composite yarns according to the method described herein; fabricating a tissue-engineered scaffold according to the methods described herein; and contacting the tissue-engineered scaffold with cells in an environment that promotes cell viability. In some embodiments, the method can further include promoting cell proliferation on the tissue-engineered scaffold.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows an example tissue engineering system 100 (shown as 100a) comprising a textile layer 102 formed of a plurality of yarns 104 in which the yarns 104 are formed of interlocking bundles of fibers 106 (not shown—see FIG. 2) to provide a scaffold for one or more substrate layers 108.

FIG. 2 shows a method 200 of fabricating a scaffold textile layer 102 in accordance with an illustrative embodiment.

FIG. 3 shows textile patterns that can be fabricated to form a pillar-shaped tissue, a mesh-shaped tissue, or a pillar mesh-shaped tissue, e.g., for vascular tissues, among others.

FIGS. 4A-4I show different example configurations of the exemplary scaffold textile layer(s) with the substrate layer(s) (e.g., one or more outer layers).

FIG. 5 shows an illustration of hierarchical structures of human skin and a designed skin scaffold. Reproduced with permission.

FIGS. 6A-6C show schematic illustrations of (6A) wet electrospinning, (6B) experimental setup for yarn fabrication, and (6C) the fibers-bundle-yarn formation process.

FIGS. 7A-7C show images of (7A) crochet process to fabricate a single chain. and (7B) crochet process to fabricate multiple chains by using different sizes of hooks. (7C) Schematic illustration of fabricating outer layers of the sandwich scaffold by electrospinning on both sides of the textile fabric for the same time duration.

FIGS. 8A-8B show graphs of (8A) the percentage of fibers aligned within ±10° along the bundle direction at different drawing speed (** represents p<0.01). (8B) Bundle diameters at different drawing speed.

FIGS. 9A-9C show photos of deposited fibers in (9A) the water bath and (9B) the ethanol bath. (9C) The surface tension of the liquid bath hinders the fiber drawing process.

FIGS. 10A-10C show (10A) Model plots by JMP Pro software for the crystallinity degree study. (10B) Schematic illustration of the crystallization process of PCL fibers. (10C) Experimental results of the crystalline degree of PCL fibers collected by different collectors (** represents p<0.01).

FIGS. 11A-11C show (11A) Fluorescent images of HUVECs (stained with green) grown on wet electrospun yarns and Petri dishes (control). (11B) The aspect ratios of cells grown on wet electrospun yarns and Petri dishes (* represents p<0.05). (11C) The percentage of cells aligned within ±10° of the direction of aligned fibers (** represents p<0.01).

FIGS. 12A-12E show tensile testing results of different components of the sandwich scaffold (12A) yarn, (12B) single chain, (12C) multiple chains, (12D) electrospun mat, (12E) sandwich scaffold. (Scaffold-20 was used since no significant difference in mechanical properties existed between Scaffold-10, Scaffold-20, and Scaffold-40).

FIGS. 13A-13B show (13A) Model plots by JMP Pro software for the mechanical properties study. (13B) Schematic illustration of the crocheting stitches of different hook sizes.

FIG. 14 shows SEM images of sandwich scaffolds before and after seeding cells.

FIG. 15 shows use the aperture card to fix the sample for the tensile testing.

FIGS. 16A-16H show fibers alignment quantification at different drawing speeds (16A) 0.2 m/s, (16B) 0.4 m/s, (16C) 0.6 m/s, (16D) 0.8 m/s, (16E) 1 m/s, (16F) 1.2 m/s, (16G) 1.4 m/s, and (16H) 1.6 m/s.

FIGS. 17A-17H show SEM images of PCL bundles fabricated at different drawing speeds (17A) 0.2 m/s, (17B) 0.4 m/s, (17C) 0.6 m/s, (17D) 0.8 m/s, (17E) 1 m/s, (17F) 1.2 m/s, (17G) 1.4 m/s, and (17H) 1.6 m/s.

FIGS. 18A-18D show cellular alignment quantification on yarns and Petri dishes (control) (18A) 4 hours, (18B) 3 days, (18C) 7 days, (18D) control.

FIG. 19 shows a table comparing applications of textile methods.

FIGS. 20A-20C show mechanical properties of textile scaffolds used in bone tissue engineering: (20A) woven scaffolds[11,14,43-45], (20B) knitted scaffolds[46], (20C) braided scaffolds[28].

FIGS. 21A-21B show mechanical properties of textile scaffolds used in cardiac tissue engineering: (21A) woven scaffolds[47], (21B) knitted scaffolds[48].

FIG. 22 shows one type of composite scaffolds which is comprised of textile fabrics and matrix have been widely used in skin tissue engineering.

FIG. 23 shows a table comparing different fiber-fabrication technologies.

FIG. 24 illustrates a research roadmap.

FIGS. 25A-25D show textile tools: (25A) home-made weaving loom, (25B) commercial knitting tool, (25C) crochet hook, (25D) commercial braiding tool.

FIG. 26 shows different textile patterns by using wet electrospun yarns.

FIGS. 27A-27B show (27A) a schematic of fabricating a vascular scaffold. (27B) Mold fabricated by 3D printing.

FIGS. 28A-28H show photos of wet electrospun PCL yarns and SEM images of wet electrospun PCL bundles fabricated from baths with different CNT concentrations: (28A, 28B) 0 mg/L, (28C, 28D) 60 mg/L, (28E, 28F) 120 mg/L, and (28G, 28H) 180 mg/L. The images of wet electrospun yarns changed from white to black as CNT concentration in the bath increased.

FIGS. 29A-29B show (29A) representative heating curves of PCL yarns fabricated from baths with different CNT concentrations. (29B) Crystalline degree of yarns fabricated from baths with different CNT concentrations (error bars represent the standard deviation, * represents p<0.05, and ** represents p<0.01). FIG. The CNT in the bath affected Thermal properties of wet electrospun yarns.

FIGS. 30A-30C show the effect of CNT in the bath on mechanical properties of wet electrospun yarns (30A) maximum stress, (30B) maximum strain, and (30C) modulus of wet electrospun yarns (error bars represent the standard deviation, * represents p<0.05, and ** represents p<0.01). Increase in galectin concentration decreased maximum stress and modulus of yarns. Mechanical properties of polymer/CNT composite yarns were greatly dependent on the content of CNTs.

FIGS. 31A-31L show SEM images of wet electrospun yarns at different degradation time points for PCL at 1 week (31A), 4 weeks (31B), and 8 weeks (31C); P8/G2 at 1 week (31D), 4 weeks (31E), and 8 weeks (31F); PCL/CNT120 at 1 week (31G), 4 weeks (31H), and 8 weeks (31I); and P8/G2/CNT120 at 1 week (31J), 4 weeks (31K), and 8 weeks (31L). The addition of CNT and gelatin accelerated the degradation of PCL yarn. The accelerated degradation rate with the addition of gelatin can be attributed to the increased hydrophilicity of PCL yarns. The accelerated degradation rate with the addition of CNT can be attributed to potentially higher enzyme binding to the CNTs in the composite yarns.

FIGS. 32A-32C show residual mass % of wet electrospun yarns PCL (32A), P9/G1 (32B), and P8/G2 (32C) at different degradation time points (error bars represent the standard deviation).

FIG. 33 show residual mass % of wet electrospun yarns after 8 weeks of degradation (error bars represent the standard deviation, * represents p<0.05).

FIG. 34 show cell viability % after cultivation for 2 days (error bars represent the standard deviation, * represents p<0.05).

FIGS. 35A-35C show (35A) Cell elongation was estimated by aspect ratio (maximum/minimum Feret diameter), and cell alignment was evaluated by the angle between the long axis of the cells and the direction of aligned fibers. (35B) Quantification of cell elongation on wet electrospun yarns after cultivation for 2 days. (35C) Quantification of cell alignment on wet electrospun yarns after cultivation for 2 days (error bars represent the standard deviation, * represents p<0.05, and ** represents p<0.01).

FIG. 36A-36C show (36A) Textile fabrics fabricated by knitting wet electrospun P8/G2/CNT120 yarns. (36B) Side view of the fabricated vascular scaffold. (36C) Front view of the fabricated vascular scaffold (scale bar: 10 mm; the textile fabrics in (36B,36C) were stained by the fluorescent dye).

FIGS. 37A-37B show representative stress-strain plots from textile tests for textile-based scaffolds and various native vessels^{75,76} in (37A) longitudinal direction and (37B) circumferential direction.

FIG. 38 shows tracking cells (red) on scaffolds fabricated by different yarns (scale bar: 200 μm; white dashed lines are the locations of yarns).

FIG. 39 shows SEM images of wet electrospun bundles fabricated from different parameters.

FIGS. 40A-40C show mechanical characterization of wet electrospun yarns. Representative stress-strain plots from the textile tests for wet electrospun yarns for PCL (40A), P9/G1 (40B), and P8/G2 (40C).

FIG. 41 shows fluorescent images of living cells (green) and dead cells (red) on wet electrospun yarns after cell cultivation for 2 days. (scale bar: 200 μm)

FIG. 42 shows fluorescent images of cells (green: Alexa Fluor, blue: DAPI) on wet electrospun yarns after cell cultivation for 2 days. (scale bar: 200 μm)

FIG. 43 illustrates a method for generating yarns with controllable mechanical properties for textile-based tissue engineering.

FIG. 44 shows a diagram of extending the solute-solvent-bath system in wet electrospinning to build a yarn database.

FIG. 45 shows a method for generating scaffolds with designable textile patterns to mimic physical, biological, and mechanical properties of human tissues.

FIG. 46 illustrates design scaffolds for vascular tissue engineering.

FIG. 47 illustrates vascular scaffold fabrication.

FIG. 48 shows a table of physical biological and mechanical requirements for human vessels and vascular scaffold.

FIG. 49 shows a table exemplifying biological requirements of scaffolds.

FIG. 50 shows a table exemplifying mechanical requirements of scaffolds.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

“Exemplary” means “an example of” and is not intended to convey an indication of a preferred or ideal aspect. “Such as” is not used in a restrictive sense, but for explanatory purposes.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

A “control” is an alternative subject or sample used in an experiment for comparison purposes. A control can be “positive” or “negative.”

The terms “culture medium” or “culture media” are to be interpreted broadly to include any medium that facilitates the growth of cell and tissues. As a non-limiting example, culture media formulations that support the growth of cells and tissue include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Endothelial Cell Growth Medium, Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's salt base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with nonessential amino acids), and the like. It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium can be from 10 to 200 units per ml. The concentration of streptomycin in the culture medium can be from 10 to 200 μg/ml. The growth factor can be fibroblast growth factor (FGF). For example, any combination of FGF10, FGF7, FGF2. The culture media may include serum of bovine or other species at a concentration at least 1% to 30%. Embryonic extract of bovine or other species can be present at a concentration of about 1% to 30%. In some embodiments, the serum can be fetal bovine serum (FBS) but other sera may be used, including horse serum or human serum. A skilled artisan will recognize that the culturing conditions can be modified to the suitable cell.

The term “electrospinning” or “electrospun,” as used herein, refers to methods where materials are streamed, sprayed, sputtered, dripped, or otherwise transported in the presence of an electric field. The electrospun material can be deposited from the direction of a charged container towards a grounded target, or from a grounded container in the direction of a charged target. In particular, the term “electrospinning” means a process in which fibers are formed from a charged solution comprising one biodegradable synthetic polymer, biopolymer, or any combination thereof by streaming the electrically charged solution through an opening or orifice towards a grounded target. The terms “solution” and “fluid” is used in the context of producing an electrospun matrix and describes a liquid that is capable of being charged.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

### Scaffolds

Described herein are scaffolds for tissue engineering formed by the methods described herein. In some embodiments, the scaffold can include: a textile layer formed of a plurality of yarns, wherein the plurality of yarns can be formed of interlocking bundles of fibers formed from a first polymer or a first polymer mixture; and one or more substrate layers including a second polymer or a second polymer mixture formed onto or attached to the textile layer. In some embodiments, the one or more substrate layers can be an outer layer. In some embodiments, the scaffold can include a second textile layer. In some embodiments, the one or more substrate layers can be positioned between a textile layer and another textile layer (e.g., as a second textile layer). In some embodiments, the second textile layer can be attached to (i) the textile layer, (ii) the one or more substrate layers of the textile layer, or any combination thereof. In some embodiments, the scaffolds for tissue engineering can include the composite yarns described herein. In some embodiments, the second textile layer can be formed of a plurality of yarns, wherein the plurality of yarns can be formed of interlocking bundles of fibers formed from a first polymer or a first polymer mixture. In some embodiments, the second textile layer can be formed of a plurality of composite yarns, wherein the plurality of composite yarns can be formed of interlocking bundles of fibers formed from a first polymer or a first polymer mixture and a carbon nanomaterial.

In some embodiments, the scaffold can include: a textile layer formed of a plurality of composite yarns, wherein the plurality of composite yarns can be formed of interlocking bundles of fibers formed from a first polymer or a first polymer mixture and a carbon nanomaterial; and one or more substrate layers including a second polymer or a second polymer mixture formed onto or attached to the textile layer. In some embodiments, the yarn can be a composite yarn described herein. In some embodiments, the fibers can further include an active agent. In some embodiments, the first polymer or first polymer mixture can further include an active agent.

In some embodiments, the fibers of the textile layer can be wet electrospun. In some embodiments, the one or more substrate layers can be wet electrospun. In some embodiments, the fibers and at least one of the one or more substrate layers can be wet electrospun. In some embodiments, the fibers and the one or more substrate layers can be wet electrospun.

In some embodiments, the one or more substrate layers can include (i) a first substrate layer that is formed of the second polymer or the second polymer mixture and (ii) a second substrate layer that is formed of a third polymer or a third polymer mixture.

In some embodiments, the scaffold can further include: a population of cells attached to the textile layer, the one or more substrate layers, or any combination thereof. In some embodiments, the population of cells can be attached to the textile layer. In some embodiments, the population of cells can be attached to the one or more substrate layers. In some embodiments, the population of cells can be attached to at least one of the one or more substrate layers. In some embodiments, the population of cells can be attached to the textile layer and the one or more substrate layers. In some embodiments, the population of cells can include, but is not limited, to connective tissue cells, organ cells, muscle cells, nerve cells, tenocytes, fibroblasts, ligament cells, endothelial cells, lung cells, epithelial cells, smooth muscle cells, cardiac muscle cells, skeletal muscle cells, islet cells, nerve cells, hepatocytes, kidney cells, bladder cells, urothelial cells, chondrocytes, bone-forming cells, induced pluripotent stem cells, adipose stem cells, bone marrow stem cells, synovium stem cells, dental pulp stem cells, neural stem cells, mesenchymal stem cells, chondrocytes, osteoblasts, myoblasts, myeloid cells, endothelial progenitor cells, any other cells from which a tissue scaffold may be generated, or any combination thereof.

In some embodiments, the scaffold can further include a coating with a biomaterial that promotes cell adhesion. In some embodiments, textile layer can be coated with a biomaterial that promotes cell adhesion. In some embodiments, the one or more substrate layer can be coated with a biomaterial that promotes cell adhesion. In some embodiments, the biomaterial can be a hydrogel. In some embodiments, the hydrogel can further include an active agent. In some embodiments, the biomaterial can include extracellular matrix components. In some embodiments, the biomaterial can include, but is not limited to gelatin, alginate, chitosan, agarose, fibrin, collagen, hyaluronic acid, or copolymers thereof, and blends thereof. In some embodiments, the biomaterial can include gelatin. In some embodiment, the scaffold can be strain-stiffened.

Suitable first polymer or the first polymer mixture, second polymer or the second polymer mixture, the third polymer or the third polymer mixture, or any combination thereof can include, but are not limited to, a biodegradable synthetic polymers (e.g., a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), a polyethylene glycol (PEG), polyurethane (PU), a poly(phosphazine), a poly(phosphate ester) or copolymers thereof, and blends thereof), a biopolymer (e.g., a gelatin, a collagen, alginate, chitosan, agarose, fibrin, hyaluronic acid, elastin, silk fibroin, or copolymers thereof, and blends thereof), or copolymers thereof, and blends thereof.

In some embodiments, the first polymer or the first polymer mixture, second polymer or the second polymer mixture, the third polymer or the third polymer mixture, or any combination thereof can include a biodegradable synthetic polymer, a biopolymer, or any combination thereof. In some embodiments, the first polymer or the first polymer mixture, second polymer or the second polymer mixture, the third polymer or the third polymer mixture, or any combination thereof can include a biodegradable synthetic polymer. In some embodiments, the first polymer or the first polymer mixture, second polymer or the second polymer mixture, the third polymer or the third polymer mixture, or any combination thereof can include a biopolymer. In some embodiments, the first polymer or the first polymer mixture, second polymer or the second polymer mixture, the third polymer or the third polymer mixture, or any combination thereof can include a biodegradable synthetic polymer and a biopolymer. In some embodiments, the fiber can include a biodegradable synthetic polymer, a biopolymer, or any combination thereof. In some embodiments, the fiber can include a biodegradable synthetic polymer. In some embodiments, the fiber can include a biopolymer. In some embodiments, the fiber can include a biodegradable synthetic polymer and a biopolymer.

In some embodiments, the fiber can include a biodegradable synthetic polymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the fiber can include a biodegradable synthetic polymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The fiber can include a biodegradable synthetic polymer in an amount ranging from any of the minimum values described above to any of the maximum values described above. For example, in some embodiments, the fiber can include a biodegradable synthetic polymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the fiber can include a biopolymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the fiber can include a biopolymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The fiber can include a biopolymer in an amount ranging from any of the minimum values described above to any of the maximum values described above. For example, in some embodiments, the fiber can include a biopolymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the fiber can include a biodegradable synthetic polymer from greater than 0% to 100% by weight and a biopolymer from 0% to 100% by weight (e.g., a biodegradable synthetic polymer from 0% to 100% by weight and a biopolymer from greater than 0% to 100% by weight, a biodegradable synthetic polymer from 0% to 10% by weight and a biopolymer from greater than 0% to 90% by weight, a biodegradable synthetic polymer from 0% to 20% by weight and a biopolymer from greater than 0% to 80% by weight, a biodegradable synthetic polymer from 0% to 30% by weight and a biopolymer from greater than 0% to 70% by weight, a biodegradable synthetic polymer from 0% to 40% by weight and a biopolymer from greater than 0% to 60% by weight, a biodegradable synthetic polymer fro 0% to 50% by weight and a biopolymer from greater than 0% to 50% by weight, a biodegradable synthetic polymer from 0% to 60% by weight and a biopolymer from greater than 0% to 40% by weight, a biodegradable synthetic polymer from 0% to 70% by weight and a biopolymer from greater than 0% to 30% by weight, a biodegradable synthetic polymer from 0% to 80% by weight and a biopolymer from greater than 0% to 20% by weight, a biodegradable synthetic polymer from 0% to 90% by weight and a biopolymer from greater than 0% to 10% by weight, a biodegradable synthetic polymer from greater than 0% to 100% by weight and a biopolymer 0%-100% by weight).

In some embodiments, the first polymer or the first polymer mixture, the second polymer or the second polymer mixture, the third polymer or the third polymer mixture, or any combination thereof can include polycaprolactone. In some embodiments, the first polymer or the first polymer mixture can include polycaprolactone. In some embodiments, the second polymer or the second polymer mixture can include polycaprolactone. In some embodiments, the third polymer mixture can include polycaprolactone. In some embodiments, the first polymer or the first polymer mixture and the second polymer or the second polymer mixture can include polycaprolactone. In some embodiments, the second polymer or the second polymer mixture and the third polymer or the third polymer mixture can include polycaprolactone. In some embodiments, the first polymer or the first polymer mixture, the second polymer or the second polymer mixture, and the third polymer or the third polymer mixture can include polycaprolactone. In some embodiments, at least one of the first polymer or the first polymer mixture, the second polymer or the second polymer mixture, the third polymer or the third polymer mixture can include polycaprolactone.

In some embodiments, a substantial portion (e.g., from 60% to 99%, from 70 5 to 99%, from 80% to 99%, or from 90% to 99%) of the wet electrospun fibers are aligned within 10 degrees along the bundle direction.

In some embodiments, the plurality of fibers can have a pre-defined mechanical property with the one or more substrate layers. In some embodiments, the pre-defined mechanical property can be selected from a pre-defined average crystalline degree, a pre-defined resilience or range, a pre-defined elastic modulus or range, a pre-defined maximum strain or range, and a pre-defined maximum stress or range, or any combination thereof.

In some embodiments, the scaffold can have a mechanical property selected from a crystalline degree between 0.2 and 0.4 (e.g., between 0.25 and 0.35), a maximum stress of between 1 MPa and 13 MPa (e.g., between 2 MPa and 13 MPa, between 1 MPa and 3 MPa, or between 3.5 MPa and 12 MPa), a maximum strain of between 0.05 and 5 (e.g., between 2 and 5, between 0.05 and 3.5, or between 0.15 and 2.5), an elastic modulus of between 1 MPa and 22 MPa (e.g., between 2 MPa and 19 MPa), and a resilience of between 0.05 MJ/m³ and 12 MJ/m³ (e.g., between 0.2 MJ/m³ and 9.5 MJ/m³), or any combination thereof.

In some embodiments, the scaffold can have a longitudinal-maximum stress of between 1 MPa and 3 MPa such as between 1.2 MPa and 1.7 MPa. In some embodiments, the scaffold can have a longitudinal-maximum strain of between 2 and 5, such as between 2 and 3, between 2 and 4. In some embodiments, the scaffold can have a circumferential-maximum stress of between 1 MPa and 3 MPa such as between 1.2 MPa and 1.7 MPa. In some embodiments, the scaffold can have a circumferential-maximum strain of between 2 and 5, such as between 3 and 5, between 4 and 5.

In some embodiments, the scaffolds may be used as a drug delivery device. In some embodiments, the scaffolds can include an active agent. In some embodiments, the scaffolds can release the active agent (e.g., a therapeutic agent such as a drug or a diagnostic agent such as a marker, dye, or other marker of that will allow visualization of a diseased state). In some examples, the scaffolds incorporate an active agent that can be released in a controlled release manner.

In some embodiments, the active agent can be incorporated onto or into the scaffold described herein by (a) coating the scaffold described herein with biomaterial such as a hydrogel further including the active agent, (b) constructing the scaffold using fibers formed from a first polymer or a first polymer mixture further including the active agent, or (c) affixing (directly or indirectly), incorporating, or impregnating the scaffold described herein with an active agent (e.g., by either a spraying process or dipping process as described above, with or without a carrier).

### Active Agents

As used herein, an “active agent” or “therapeutic agent” refers to one or more therapeutic agents, active ingredients, or substances that can be used to treat a medical condition of the eye, a cancer, heart, or contraception. As discussed herein, the therapeutic agents can be released from the disclosed scaffolds in a biologically active form.

It is further understood, that as used herein, the term “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term, therefore, encompasses those compounds or chemicals traditionally regarded as drugs, vaccines, and biopharmaceuticals, including molecules such as proteins, peptides, hormones, nucleic acids, gene constructs, and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary applications, and in agriculture, such as with plants, as well as other areas. The term therapeutic agent also includes, without limitations, medicaments; vitamins; mineral supplements; substances used for the treatment, prevention, diagnosis, cure, or mitigation of disease or illness; or substances which affect the structure or function of the body; or pro-drugs, which become biologically active or more active after they have been placed in a predetermined physiological environment.

In some embodiments, the therapeutic agent may comprise an agent useful as a contraceptive such as a progestins, estrogens, or any combination thereof. For example, suitable progestins include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. For example, suitable estrogens include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators.

In some embodiments, the therapeutic agent may comprise gonadotropin releasing hormone (GnRh) or anologs thereof such as deslorelin, avorelin, leuprolide, triptorelin, nafarelin, goserelin, buserelin, and fertirelin.

In some embodiments, the therapeutic agent may comprise an agent useful in the treatment of an ophthalmological disorder or an eye disease such as: beta-blockers including timolol, betaxolol, levobetaxolol, and carteolol; miotics including pilocarpine; carbonic anhydrase inhibitors; serotonergics; muscarinics; dopaminergic agonists; adrenergic agonists including apraclonidine and brimonidine; anti-angiogenesis agents; anti-infective agents including quinolones such as ciprofloxacin and aminoglycosides such as tobramycin and gentamicin; non-steroidal and steroidal anti-inflammatory agents, such as suprofen, diclofenac, ketorolac, rimexolone, and tetrahydrocortisol; growth factors, such as EGF; immunosuppressant agents; and anti-allergic agents including olopatadine; prostaglandins such as latanoprost; 15-keto latanoprost; travoprost; and unoprostone isopropyl.

In some embodiments, the therapeutic agent is selected from the group consisting of an anti-inflammatory agent, a calcineurin inhibitor, an antibiotic, a nicotinic acetylcholine receptor agonist, and an anti-lymphangiogenic agent. In some embodiments, the anti-inflammatory agent may be cyclosporine. In some embodiments, the calcineurin inhibitor may be voclosporin. In some embodiments, the antibiotic may be selected from the group consisting of amikacin, gentamycin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, and tetracycline. In some embodiments, the nicotinic acetylcholine receptor agonist may be any of pilocarpine, atropine, nicotine, epibatidine, lobeline, or imidacloprid. In some embodiments, the anti-lymphangiogenic agent may be a vascular endothelial growth factor C (VEGF-C) antibody, a VEGF-D antibody, or a VEGF-3 antibody.

In some aspects, the therapeutic agent may be selected from: a beta-blocker, including levobunolol (BETAGAN), timolol (BETIMOL, TIMOPTIC), betaxolol (BETOPTIC), and metipranolol (OPTIPRANOLOL); alpha-agonists, such as apraclonidine (IOPIDINE) and brimonidine (ALPHAGAN); carbonic anhydrase inhibitors, such as acetazolamide, methazolamide, dorzolamide (TRUSOPT) and brinzolamide (AZOPT); prostaglandins or prostaglandin analogs such as latanoprost (XALATAN), bimatoprost (LUMIGAN) and travoprost (TRAVATAN); miotic or cholinergic agents, such as pilocarpine (ISOPTO CARPINE, PILOPINE) and carbachol (ISOPTO CARBACHOL); epinephrine compounds, such as dipivefrin (PROPINE); forskolin; or neuroprotective compounds, such as brimonidine and memantine; a steroid derivative, such as 2-methoxyestradiol or analogs or derivatives thereof, or an antibiotic.

The term “VEGF” refers to a vascular endothelial growth factor that induces angiogenesis or an angiogenic process, including, but not limited to, increased permeability. As used herein, the term “VEGF” includes the various subtypes of VEGF (also known as vascular permeability factor (VPF) and VEGF-A) that arise by, e.g., alternative splicing of the VEGF-A/VPF gene including VEGF121, VEGF165, and VEGF189. Further, as used herein, the term “VEGF” includes VEGF-related angiogenic factors such as PIGF (placental growth factor), VEGF-B, VEGF-C, VEGF-D, and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1), VEGFR-2 (KDR/Flk-1), or VEGFR-3 (FLT-4). The term “VEGF” can be used to refer to a “VEGF” polypeptide or a “VEGF” encoding gene or nucleic acid.

The term “anti-VEGF agent” refers to an agent that reduces, or inhibits, either partially or fully, the activity or production of a VEGF. An anti-VEGF agent can directly or indirectly reduce or inhibit the activity or production of a specific VEGF such as VEGF165. Furthermore, “anti-VEGF agents” include agents that act on either a VEGF ligand or its cognate receptor so as to reduce or inhibit a VEGF-associated receptor signal. Non-limiting examples of “anti-VEGF agents” include antisense molecules, ribozymes, or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

In some embodiments, the therapeutic agent may comprise an anti-VEGF agent. Representative examples of anti-VEGF agents include ranibizumab, bevacizumab, aflibercept, KH902 VEGF receptor-Fc, fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-, hydroxyisovalerylshikonin, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, G6-31 antibody, a fusion antibody and an antibody that binds to an epitope of VEGF. Additional non-limiting examples of anti-VEGF agents useful in the present methods include a substance that specifically binds to one or more of a human vascular endothelial growth factor-A (VEGF-A), human vascular endothelial growth factor-B (VEGF-B), human vascular endothelial growth factor-C (VEGF-C), human vascular endothelial growth factor-D (VEGF-D) and human vascular endothelial growth, factor-E (VEGF-E), and an antibody that binds, to an epitope of VEGF.

In various aspects, the anti-VEGF agent is the antibody ranibizumab or a pharmaceutically acceptable salt thereof. Ranibizumab is commercially available under the trademark LUCENTIS. In another embodiment, the anti-VEGF agent is the antibody bevacizumab or a pharmaceutically acceptable salt thereof. Bevacizumab is commercially available under the trademark AVASTIN. In another embodiment, the anti-VEGF agent is aflibercept or a pharmaceutically acceptable salt thereof. Aflibercept is commercially available under the trademark EYLEA. In one embodiment, the anti-VEGF agent is pegaptanib or a pharmaceutically acceptable salt thereof. Pegaptinib is commercially available under the trademark MACUGEN. In another embodiment, the anti-VEGF agent is an antibody or an antibody fragment that binds to an epitope of VEGF, such as an epitope of VEGF-A, VEGF-B, VEGF-C, VEGF-D, or VEGF-E. In some embodiments, the VEGF antagonist binds to an epitope of VEGF such that the binding of VEGF and VEGFR are inhibited. In one embodiment, the epitope encompasses a component of the three-dimensional structure of VEGF that is displayed, such that the epitope is exposed on the surface of the folded VEGF molecule. In one embodiment, the epitope is a linear amino acid sequence from VEGF.

In various aspects, the therapeutic agent may comprise an agent that blocks or inhibits VEGF-mediated activity, e.g., one or more VEGF antisense nucleic acids. The present disclosure provides the therapeutic or prophylactic use of nucleic acids comprising at least six nucleotides that are antisense to a gene or cDNA encoding VEGF or a portion thereof. As used herein, a VEGF “antisense” nucleic acid refers to a nucleic acid capable of hybridizing by virtue of some sequence complementarity to a portion of an RNA (preferably mRNA) encoding VEGF. The antisense nucleic acid may be complementary to a coding and/or noncoding region of an mRNA encoding VEGF. Such antisense nucleic acids have utility as compounds that prevent VEGF expression and can be used in the treatment of diabetes. The antisense nucleic acids of the disclosure are double-stranded or single-stranded oligonucleotides, RNA or DNA or a modification or derivative thereof, and can be directly administered to a cell or produced intracellularly by transcription of exogenous, introduced sequences.

The VEGF antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisense molecules include short double-stranded RNAs, known as siRNAs, and short hairpin RNAs, and long dsRNA (>50 bp but usually ≥500 bp).

In various aspects, the therapeutic agent may comprise one or more ribozyme molecules designed to catalytically cleave gene mRNA transcripts encoding VEGF, preventing translation of target gene mRNA and, therefore, expression of the gene product.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA and must include the well-known catalytic sequence responsible for mRNA cleavage. For this sequence, see, e.g., U.S. Pat. No. 5,093,246. While ribozymes that cleave mRNA at site-specific recognition sequences can be used to destroy mRNAs encoding VEGF, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes are well known in the art. The ribozymes of the present disclosure also include RNA endoribonucleases (hereinafter “Cech-type ribozymes”), such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place. The disclosure encompasses those Cech-type ribozymes that target eight base-pair active site sequences that are present in the gene encoding VEGF.

In further aspects, the therapeutic agent may comprise an antibody that inhibits VEGF, such as bevacizumab or ranibizumab. In still further aspects, the therapeutic agent may comprise an agent that inhibits VEGF activity, such as a tyrosine kinases stimulated by VEGF, examples of which include, but are not limited to lapatinib, sunitinib, sorafenib, axitinib, and pazopanib.

The term “anti-RAS agent” or “anti-Renin Angiotensin System agent” refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

In some embodiments, the therapeutic agent may comprise a renin-angiotensin system (RAS) inhibitor. In some embodiments, the renin-angiotensin system (RAS) inhibitor is one or more of an angiotensin-converting enzyme (ACE) inhibitor, an angiotensin-receptor blocker, and a renin inhibitor.

Non limiting examples of angiotensin-converting enzyme (ACE) inhibitors which are useful in the present invention include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof.

Non-limiting examples of angiotensin-receptor blockers which are useful in the present invention include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

Non-limiting examples of renin inhibitors which may be used as therapeutic agents include, but are not limited to: aliskiren, ditekiren, enalkiren, remikiren, terlakiren, ciprokiren and zankiren, pharmaceutically acceptable salts thereof, and mixtures thereof.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methyl-prednisolone, triamcinolone, fluocinolone, aldosterone, spironolactone, danazol (otherwise known as OPTINA), and others. In some embodiments, the therapeutic agent may comprise a steroid.

The terms “peroxisome proliferator-activated receptor gamma agent,” or “PPAR-7 agent,” or “PPARG agent,” or “PPAR-gamma agent” refer to agents which directly or indirectly act upon the peroxisome proliferator-activated receptor. This agent may also influence PPAR-alpha, “PPARA” activity.

In some embodiments, the therapeutic agent may comprise a modulator of macrophage polarization. Illustrative modulators of macrophage polarization include peroxisome proliferator activated receptor gamma (PPAR-g) modulators, including, for example, agonists, partial agonists, antagonists, or combined PPAR-gamma/alpha agonists. In some embodiments, the therapeutic agent may comprise a PPAR gamma modulator, including PPAR gamma modulators that are full agonists or partial agonists. In some embodiments, the PPAR gamma modulator is a member of the drug class of thiazolidinediones (TZDs, or glitazones). By way of non-limiting example, the PPAR gamma modulator may be one or more of rosiglitazone (AVANDIA), pioglitazone (ACTOS), troglitazone (REZULIN), netoglitazone, rivoglitazone, ciglitazone, rhodanine. In some embodiments, the PPAR gamma modulator is one or more of irbesartan and telmesartan. In some embodiments, the PPAR gamma modulator is a nonsteroidal anti-inflammatory drug (NSAID, such as, for example, ibuprofen) or an indole. Known inhibitors include the experimental agent GW-9662. Further examples of PPAR gamma modulators are described in WIPO Publication Nos. WO/1999/063983, WO/2001/000579, Nat Rev Immunol. 2011 Oct. 25; 11(11):750-61, or agents identified using the methods of WO/2002/068386, the contents of which are hereby incorporated by reference in their entireties.

In some embodiments, the PPAR gamma modulator is a “dual,” or “balanced,” or “pan” PPAR modulator. In some embodiments, the PPAR gamma modulator is a glitazar, which bind two or more PPAR isoforms, e.g., muraglitazar (Pargluva) and tesaglitazar (Galida) and aleglitazar.

In some embodiments, the therapeutic agent may comprise semapimod (CNI-1493) as described in Bianchi, et al. (March 1995). Molecular Medicine (Cambridge, Mass.) 1 (3): 254-266, the contents of which is hereby incorporated by reference in its entirety.

In some embodiments, the therapeutic agent may comprise a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7,294,753 the contents of which are hereby incorporated by reference in their entireties. In some embodiments, the MIF inhibitor is (S,R)-3-(4-hydroxyphenyl)-4,5-dihydro-5-isoxazole acetic acid methyl ester (ISO-1), isoxazoline, p 425 (J. Biol. Chem., 287, 30653-30663), epoxyazadiradione, or vitamin E.

In some embodiments, the therapeutic agent may comprise a chemokine receptor 2 (CCR2) inhibitor as described in, for example, U.S. patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872, CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH351125, TAK779, Teijin, RS-504393, compound 2, compound 14, or compound 19 (Plos ONE 7(3): e32864).

In some embodiments, the therapeutic agent may comprise an agent that modulates autophagy, microautophagy, mitophagy, or other forms of autophagy. In some embodiments, the therapeutic agent may comprise sirolimus, tacrolimis, rapamycin, everolimus, bafilomycin, chloroquine, hydroxychloroquine, spautin-1, metformin, perifosine, resveratrol, trichostatin, valproic acid, Z-VAD-FMK, or others known to those in the art. Without wishing to be bound by theory, agent that modulates autophagy, microautophagy, mitophagy or other forms of autophagy may alter the recycling of intracellular components, for example, but not limited to, cellular organelles, mitochondria, endoplasmic reticulum, lipid or others. Without further wishing to be bound by theory, this agent may or may not act through microtubule-associated protein 1A/1B-light chain 3 (LC3).

In some embodiments, the therapeutic agent may comprise an agent used to treat cancer, i.e., a cancer drug or anti-cancer agent. Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells, such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect, the antimitotic agent is a taxane. More preferably in this aspect, the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane), and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, histrelin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, rnedroxyprogesterone acetate, and megestrol acetate; estrogens such as diethylstilbestrol and ethinyl estradiol, and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade names of these and additional examples of cancer drugs, and their methods of use, including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation-inducing agents, cell growth arrest inducing agents, apoptosis-inducing agents, cytotoxic agents, agents affecting cell bioenergetics, i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

According to certain aspects, the therapeutic agent may comprise a biologic drug, particularly an antibody. According to some aspects, the antibody is selected from the group consisting of cetuximab, anti-CD24 antibody, panitumumab, and bevacizumab.

Therapeutic agents as used in the present disclosure may comprise peptides, proteins such as hormones, enzymes, antibodies, monoclonal antibodies, antibody fragments, monoclonal antibody fragments, and the like, nucleic acids such as aptamers, siRNA, DNA, RNA, antisense nucleic acids or the like, antisense nucleic acid analogs or the like, low-molecular-weight compounds, or high-molecular-weight compounds, receptor agonists, receptor antagonists, partial receptor agonists, and partial receptor antagonists.

Additional representative therapeutic agents may include, but are not limited to, peptide drugs, protein drugs, desensitizing materials, antigens, factors, growth factors, anti-infective agents such as antibiotics, antimicrobial agents, antiviral, antibacterial, antiparasitic, antifungal substances and a combination thereof, antiallergenics, steroids, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, anti-Alzheimer's agents, antihypertensive agents, beta-adrenergic blocking agents, alpha-adrenergic blocking agents, nutritional agents, and the benzophenanthridine alkaloids. The therapeutic agent can further be a substance capable of acting as a stimulant, a sedative, a hypnotic, an analgesic, an anticonvulsant, and the like.

Additional therapeutic agents may comprise CNS-active drugs, neuro-active drugs, inflammatory and anti-inflammatory drugs, renal and cardiovascular drugs, gastrointestinal drugs, anti-neoplastics, immunomodulators, immunosuppressants, hematopoietic agents, growth factors, anticoagulant, thrombolytic, antiplatelet agents, hormones, hormone-active agents, hormone antagonists, vitamins, ophthalmic agents, anabolic agents, antacids, anti-asthmatic agents, anti-cholesterolemic and anti-lipid agents, anti-convulsants, anti-diarrheals, anti-emetics, anti-manic agents, antimetabolite agents, anti-nauseants, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, laxatives, mineral supplements, mucolytic agents, neuromuscular drugs, peripheral vasodilators, psychotropics, stimulants, thyroid and anti-thyroid agents, tissue growth agents, uterine relaxants, vitamins, antigenic materials, and so on. Other classes of therapeutic agents include those cited in Goodman & Gilman's The Pharmacological Basis of Therapeutics (McGraw Hill) as well as therapeutic agents included in the Merck Index and The Physicians' Desk Reference (Thompson Healthcare).

Other therapeutic agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Further examples of therapeutic agents include, but are not limited to, peptide drugs, protein drugs, desensitizing materials, antigens, anti-infective agents such as antibiotics, antimicrobial agents, antiviral, antibacterial, antiparasitic, antifungal substances, and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, antiproliferatives, anti-VEGF agents, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

Further representative therapeutic agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, paclitaxel, taxol, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The therapeutic agent can also be an immunomodulator, including, for example, cytokines, interleukins, interferon, colony stimulating factor, tumor necrosis factor, and the like; immunosuppressants such as rapamycin, tacrolimus, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcus neoformans, Histoplasma capsulatum, Candida albicans, Candida tropicalis, Nocardia asteroids, Rickettsia ricketsii, Rickettsia typhi, Mycoplasma pneumoniae, Chlamydia psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the therapeutic agent can comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the therapeutic agent can be a combination of Rifampicin (Rifampin in U.S.) and Minocycline.

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Cytokines useful as therapeutic agents include, but are not limited to, cardiotrophin, stromal cell derived factor, macrophage derived chemokine (MDC), melanoma growth stimulatory activity (MGSA), macrophage inflammatory proteins 1 alpha (MIP-1alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, and TNF-β. Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as therapeutic agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12, 13, 15, 16, 17 and 18.

### Methods of Making Scaffolds

Described herein are methods of making a scaffold for tissue engineering including providing a textile layer formed of a plurality of yarns, wherein the plurality of yarns are formed of interlocking bundles of fibers formed from a first polymer or a first polymer mixture; and forming one or more substrate layer of a second polymer or a second polymer mixture onto the textile layer having a pre-defined thickness. In some embodiments, the plurality of yarns can be a plurality of composite yarns described herein. In some embodiments, the fibers can further include an active agent. In some embodiments, the first polymer or first polymer mixture can further include an active agent.

In some embodiments, the method of making a scaffold for tissue engineering can include: fabricating composite yarns according to the methods described herein; and forming a scaffold including the composite yarns described herein. In some embodiments, forming the scaffold can include providing a textile layer formed of a plurality of composite yarns, wherein the plurality of composite yarns can be formed of interlocking bundles of fibers formed from a first polymer or a first polymer mixture and a carbon nanomaterial. In some embodiments, when the scaffold includes composite yarns, the one or more substrate layer is not present. In some embodiments, when the scaffold includes composite yarns, the one or more substrate layer is present. In some embodiments, forming the scaffold can include providing a textile layer formed of a plurality of composite yarns, wherein the plurality of composite yarns can be formed of interlocking bundles of fibers formed from a first polymer or a first polymer mixture and a carbon nanomaterial; and forming one or more substrate layer of a second polymer or a second polymer mixture onto the textile layer having a pre-defined thickness. In some embodiments, forming the scaffold can include a weaving operation, a knitting operation, a crocheting operation, a knotting operation, a tatting operation, a felting operation, a bonding operation, or a braiding operation to have a pre-defined textile pattern for cell growth of a population of cells onto the scaffold.

In some embodiments, the textile layer can be formed of the plurality of yarns by a weaving operation, a knitting operation, a crocheting operation, a knotting operation, a tatting operation, a felting operation, a bonding operation, or a braiding operation to have a pre-defined textile pattern for cell growth of a population of cells onto the scaffold. In some embodiments, forming the textile layer can include crocheting the plurality of yarns with a pre-defined crochet hook size to provide the scaffold with a pre-defined mechanical property selected from a pre-defined resilience or range, a pre-defined elastic modulus or range, a pre-defined maximum strain or range, a pre-defined maximum stress or range, or any combination thereof. In some embodiments, forming the textile layer can include coating the plurality of yarns with a biomaterial that promotes cell adhesion.

In some embodiments, the method can include forming a second textile layer. In some embodiments, the second textile layer can be formed of a plurality of yarns, wherein the plurality of yarns can be formed of interlocking bundles of fibers formed from a first polymer or a first polymer mixture. In some embodiments, the second textile layer can be formed of a plurality of composite yarns, wherein the plurality of composite yarns can be formed of interlocking bundles of fibers formed from a first polymer or a first polymer mixture and a carbon nanomaterial. In some embodiments, the one or more substrate layers can be positioned between a textile layer and another textile layer (e.g., as a second textile layer). In some embodiments, the second textile layer can be attached to (i) the textile layer, (ii) the one or more substrate layers of the textile layer, or any combination thereof. In some embodiments, the scaffolds for tissue engineering can include the composite yarns described herein.

In some embodiments, the method can further include: forming a yarn of the plurality of fibers by electrospinning the first polymer or the first polymer mixture into the plurality of fibers; drawing the plurality of fibers out of an electrospinning bath to form the bundles; and forming the yarn from the bundles. In some embodiments, electrospinning of the fiber can include wet electrospinning. In some embodiments, the one or more substrate layer can be an electrospun mat.

In some embodiments, the step of drawing the plurality of fibers out of the electrospinning bath to form the bundles can further include winding the plurality of drawn fibers around a roller. In some embodiments, the method can further include varying a drawing speed to generate (i) a desired fiber alignment of the plurality of fibers, (ii) a desired bundle diameter of the plurality of fibers, or a combination thereof. In some embodiments, the method can further include seeding a population of cells onto the scaffold.

In some embodiments, the method can further include: electrospinning the one or more substrate layers; and varying the duration of the electrospinning step to achieve a desired substrate layer thickness. In some embodiments, the electrospinning bath can include a liquid having a concentration in the electrospinning bath to form the plurality of fibers having a pre-defined mechanical property with the one or more substrate layers.

In some embodiments, the pre-defined mechanical property can be selected from a pre-defined resilience or range, a pre-defined elastic modulus or range, a pre-defined maximum strain or range, and a pre-defined maximum stress or range, or any combination thereof. In some embodiments, the liquid concentration can be varied to generate the plurality of fibers having a pre-defined average crystalline degree.

In some embodiments, the scaffold can have a mechanical property selected from a crystalline degree between 0.2 and 0.4 (e.g., between 0.25 and 0.35), a maximum stress of between 1 MPa and 13 MPa (e.g., between 2 MPa and 13 MPa, between 1 MPa and 3 MPa, or between 3.5 MPa and 12 MPa), a maximum strain of between 0.05 and 5 (e.g., between 2 and 5, between 0.05 and 3.5, or between 0.15 and 2.5), an elastic modulus of between 1 MPa and 22 MPa (e.g., between 2 MPa and 19 MPa), and a resilience of between 0.05 MJ/m³ and 12 MJ/m³ (e.g., between 0.2 MJ/m³ and 9.5 MJ/m³), or any combination thereof.

In some embodiments, the scaffold can have a longitudinal maximum stress of between 1 MPa and 3 MPa such as between 1.2 MPa and 1.7 MPa. In some embodiments, the scaffold can have a longitudinal maximum strain of between 2 and 5, such as between 2 and 3, between 2 and 4. In some embodiments, the scaffold can have a circumferential maximum stress of between 1 MPa and 3 MPa such as between 1.2 MPa and 1.7 MPa. In some embodiments, the scaffold can have a circumferential maximum strain of between 2 and 5, such as between 3 and 5, between 4 and 5.

Suitable electrospinning bath liquid can include but is not limited to water, culture media, alcohol (e.g., ethanol, propanol, methanol, butanol, pentanol, hexanol, or any combination thereof), or any combination thereof. In some embodiments, the liquid can include an alcohol. In some embodiments, the liquid can include water. In some embodiments, the liquid can include culture media. In some embodiments, the liquid can be ethanol. In some embodiments, the liquid can be propanol. In some embodiments, the liquid can include water and an alcohol. In some embodiments, the electrospinning bath liquid can include an ethanol in an amount of greater than 40% (e.g., greater than 50%, greater than 60%, greater than 70%, greater than 80%, or greater than 90%). In some embodiments, the electrospinning bath liquid can include an ethanol in an amount of 100% or less (e.g., 90% or less, 80% or less, 70% or less, 60% or less, or 50% or less).

The electrospinning bath liquid can include an ethanol in an amount ranging from any of the minimum values described above to any of the maximum values described above. For example, in some embodiments, the electrospinning bath liquid can include an ethanol in an amount of from 40% to 100% (e.g., from 40% to 90%, from 40% to 80%, from 40% to 70%, from 40% to 60%, from 40% to 50%, from 50% to 100%, from 50% to 90%, from 50% to 80%, from 50% to 70%, from 50% to 60%, from 60% to 100%, from 60% to 90%, from 60% to 80%, from 60% to 70%, from 70% to 100%, from 70% to 90%, from 70% to 80%, from 80% to 100%, from 80% to 90%, or from 90% to 100%). In some embodiments, the electrospinning bath can further include a carbon nanomaterial to generate the plurality of fibers with a carbon nanomaterial coating. In some embodiments, the carbon nanomaterial can include carbon nanotubes, graphene, carbon dots, or any combination thereof. In some embodiments, the carbon nanomaterial can include carbon nanotubes. In some embodiments, the carbon nanomaterial can include graphene. In some embodiments, the carbon nanomaterial can include carbon dots.

In some embodiments, the carbon nanomaterial in the electrospinning bath can be present in an amount of at least greater than 0 mg/L (e.g., at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, or at least 190 mg/L). In some embodiments, the carbon nanomaterial in the electrospinning bath can be present in an amount of 200 mg/L or less (e.g., 190 mg/L or less, 180 mg/L or less, 170 mg/L or less, 160 mg/L or less, 150 mg/L or less, 140 mg/L or less, 130 mg/L or less, 120 mg/L or less, 110 mg/L or less, 100 mg/L or less, 90 mg/L or less, 80 mg/L or less, 70 mg/L or less, 60 mg/L or less, 50 mg/L or less, 40 mg/L or less, 30 mg/L or less, 20 mg/L or less, 10 mg/L or less, or 5 mg/L or less).

The carbon nanomaterial in the electrospinning bath can be present in an amount ranging from any of the minimum values described above to any of the maximum values described above. For example, in some embodiments, the carbon nanomaterial in the electrospinning bath can be present in an amount of from greater than 0 mg/L to 200 mg/L (e.g., from greater than 0 mg/L to 180 mg/L, from greater than 0 mg/L to 160 mg/L, from greater than 0 mg/L to 140 mg/L, from greater than 0 mg/L to 100 mg/L, from greater than 0 mg/L to 50 mg/L, from greater than 0 mg/L to 25 mg/L, from greater than 0 mg/L to 10 mg/L, from 5 mg/L to 10 mg/L, from 5 mg/L to 25 mg/L, from 5 mg/L to 50 mg/L, from 5 mg/L to 100 mg/L, from 5 mg/L to 150 mg/L, from 5 mg/L to 200 mg/L, from 10 mg/L to 25 mg/L, from 10 mg/L to 50 mg/L, from 10 mg/L to 100 mg/L, from 10 mg/L to 150 mg/L, from 10 mg/L to 200 mg/L, from 25 mg/L to 50 mg/L, from 25 mg/L to 100 mg/L, from 25 mg/L to 150 mg/L, from 25 mg/L to 200 mg/L, from 50 mg/L to 100 mg/L, from 50 mg/L to 150 mg/L, from 50 mg/L to 200 mg/L, from 100 mg/L to 150 mg/L, from 100 mg/L to 200 mg/L, or from 150 mg/L to 200 mg/L).

In some embodiments, the one or more substrate layers can include (i) a first substrate layer that is formed of the second polymer or the second polymer mixture and (ii) a second substrate layer that is formed of a third polymer or a third polymer mixture.

Suitable first polymer or the first polymer mixture, second polymer or the second polymer mixture, the third polymer or the third polymer mixture, or any combination thereof can include, but are not limited to, a biodegradable synthetic polymers (e.g., a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), a polyethylene glycol (PEG), polyurethane (PU), a poly(phosphazine), a poly(phosphate ester) or copolymers thereof, and blends thereof), a biopolymer (e.g., a gelatin, a collagen, alginate, chitosan, agarose, fibrin, hyaluronic acid, elastin, silk fibroin, or copolymers thereof, and blends thereof), or copolymers thereof, and blends thereof.

In some embodiments, the first polymer or the first polymer mixture, second polymer or the second polymer mixture, the third polymer or the third polymer mixture, or any combination thereof can include a biodegradable synthetic polymer, a biopolymer, or any combination thereof. In some embodiments, the first polymer or the first polymer mixture, second polymer or the second polymer mixture, the third polymer or the third polymer mixture, or any combination thereof can include a biodegradable synthetic polymer. In some embodiments, the first polymer or the first polymer mixture, second polymer or the second polymer mixture, the third polymer or the third polymer mixture, or any combination thereof can include a biopolymer. In some embodiments, the first polymer or the first polymer mixture, second polymer or the second polymer mixture, the third polymer or the third polymer mixture, or any combination thereof can include a biodegradable synthetic polymer and a biopolymer. In some embodiments, the fiber can include a biodegradable synthetic polymer, a biopolymer, or any combination thereof. In some embodiments, the fiber can include a biodegradable synthetic polymer. In some embodiments, the fiber can include a biopolymer. In some embodiments, the fiber can include a biodegradable synthetic polymer and a biopolymer.

In some embodiments, the fiber can include a biodegradable synthetic polymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the fiber can include a biodegradable synthetic polymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The fiber can include a biodegradable synthetic polymer in an amount ranging from any of the minimum values described above to any of the maximum values described above. For example, in some embodiments, the fiber can include a biodegradable synthetic polymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the fiber can include a biopolymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the fiber can include a biopolymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The fiber can include a biopolymer in an amount ranging from any of the minimum values described above to any of the maximum values described above. For example, in some embodiments, the fiber can include a biopolymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the fiber can include a biodegradable synthetic polymer from greater than 0% to 100% by weight and a biopolymer from 0% to 100% by weight (e.g., a biodegradable synthetic polymer from 0% to 100% by weight and a biopolymer from greater than 0% to 100% by weight, a biodegradable synthetic polymer from 0% to 10% by weight and a biopolymer from greater than 0% to 90% by weight, a biodegradable synthetic polymer from 0% to 20% by weight and a biopolymer from greater than 0% to 80% by weight, a biodegradable synthetic polymer from 0% to 30% by weight and a biopolymer from greater than 0% to 70% by weight, a biodegradable synthetic polymer from 0% to 40% by weight and a biopolymer from greater than 0% to 60% by weight, a biodegradable synthetic polymer fro 0% to 50% by weight and a biopolymer from greater than 0% to 50% by weight, a biodegradable synthetic polymer from 0% to 60% by weight and a biopolymer from greater than 0% to 40% by weight, a biodegradable synthetic polymer from 0% to 70% by weight and a biopolymer from greater than 0% to 30% by weight, a biodegradable synthetic polymer from 0% to 80% by weight and a biopolymer from greater than 0% to 20% by weight, a biodegradable synthetic polymer from 0% to 90% by weight and a biopolymer from greater than 0% to 10% by weight, a biodegradable synthetic polymer from greater than 0% to 100% by weight and a biopolymer 0%-100% by weight).

In some embodiments, the first polymer or the first polymer mixture, the second polymer or the second polymer mixture, the third polymer or the third polymer mixture, or any combination thereof can include polycaprolactone. In some embodiments, the first polymer or the first polymer mixture can include polycaprolactone. In some embodiments, the second polymer or the second polymer mixture can include polycaprolactone. In some embodiments, the third polymer mixture can include polycaprolactone. In some embodiments, the first polymer or the first polymer mixture and the second polymer or the second polymer mixture can include polycaprolactone. In some embodiments, the second polymer or the second polymer mixture and the third polymer or the third polymer mixture can include polycaprolactone. In some embodiments, the first polymer or the first polymer mixture, the second polymer or the second polymer mixture, and the third polymer or the third polymer mixture can include polycaprolactone. In some embodiments, at least one of the first polymer or the first polymer mixture, the second polymer or the second polymer mixture, the third polymer or the third polymer mixture can include polycaprolactone.

In some embodiments, the method can further include seeding a population of cells onto the scaffold. In some embodiments, the population of cells can include, but is not limited to connective tissue cells, organ cells, muscle cells, nerve cells, tenocytes, fibroblasts, ligament cells, endothelial cells, lung cells, epithelial cells, smooth muscle cells, cardiac muscle cells, skeletal muscle cells, islet cells, nerve cells, hepatocytes, kidney cells, bladder cells, urothelial cells, chondrocytes, bone-forming cells, induced pluripotent stem cells, adipose stem cells, bone marrow stem cells, synovium stem cells, dental pulp stem cells, neural stem cells, mesenchymal stem cells, chondrocytes, osteoblasts, myoblasts, myeloid cells, endothelial progenitor cells, any other cells from which a tissue scaffold may be generated, or any combination thereof.

In some embodiments, forming the textile layer can include coating the plurality of yarns with a biomaterial that promotes cell adhesion. In some embodiments, forming the one or more substrate layer can include coating one or more substrate layers with a biomaterial that promotes cell adhesion. In some embodiments, forming the scaffold can include coating the plurality of yarns, the one or more substrate layer, or any combination thereof with a biomaterial that promotes cell adhesion. In some embodiments, the biomaterial can be a hydrogel. In some embodiments, the hydrogel can further include an active agent. In some embodiments, the biomaterial can include extracellular matrix components. In some embodiments, the biomaterial can include, but is not limited to, gelatin, alginate, chitosan, agarose, fibrin, collagen, hyaluronic acid, copolymers thereof, and blends thereof. In some embodiments, the biomaterial can include gelatin. In some embodiments, the biomaterial can include collagen.

In some embodiments, forming the scaffold can include providing a textile layer formed of a plurality of composite yarns, wherein the plurality of composite yarns can be formed of interlocking bundles of fibers formed from a first polymer or a first polymer mixture and a carbon nanomaterial; and coating the plurality of composite yarns with a biomaterial that promotes cell adhesion.

In some embodiments, forming the scaffold can include providing a textile layer formed of a plurality of composite yarns, wherein the plurality of composite yarns can be formed of interlocking bundles of fibers formed from a first polymer or a first polymer mixture and a carbon nanomaterial; forming one or more substrate layer of a second polymer or a second polymer mixture onto the textile layer having a pre-defined thickness; and coating the plurality of yarns, the one or more substrate layer, or any combination thereof with a biomaterial that promotes cell adhesion.

### Electrospinning

The process of electrospinning generally involves the creation of an electrical field at the surface of a liquid. The resulting electrical forces create a jet of liquid which carries electrical charge. The liquid jets may be attracted to other electrically charged objects at a suitable electrical potential. As the jet of liquid elongates and travels, it will harden and dry. The hardening and drying of the elongated jet of liquid may be caused by cooling of the liquid, i.e., where the liquid is normally a solid at room temperature; evaporation of a solvent, e.g., by dehydration, (physically induced hardening); or by a curing mechanism (chemically induced hardening). The produced fibers are collected on a suitably located, oppositely charged target substrate.

The electrospinning apparatus can include an electrodepositing mechanism and a target substrate. The electrodepositing mechanism includes at least one container to hold the solution that is to be electrospun. The container has at least one orifice or nozzle to allow the streaming of the solution from the container. If there are multiple containers, a plurality of nozzles may be used. One or more pumps (e.g., a syringe pump) used in connection with the container can be used to control the flow of solution streaming from the container through the nozzle. The pump can be programmed to increase or decrease the flow at different points during electrospinning.

The electrospinning occurs due to the presence of a charge in either the orifices or the target, while the other is grounded. In some embodiments, the nozzle or orifice is charged and the target is grounded. Those of skill in the electrospinning arts will recognize that the nozzle and solution can be grounded and the target can be electrically charged.

The target can also be specifically charged or grounded along a preselected pattern so that the solution streamed from the orifice is directed in specific directions. The electric field can be controlled by a microprocessor to create an electrospun matrix having a desired geometry. The target and the nozzle or nozzles can be engineered to be movable with respect to each other, thereby allowing additional control over the geometry of the electrospun matrix to be formed. The entire process can be controlled by a microprocessor that is programmed with specific parameters that will obtain a specific preselected electrospun matrix.

Minimal electrical current is involved in the electrospinning process; therefore, the process does not denature the materials that form the electrospun matrix, because the current causes little or no temperature increase in the solutions during the procedure.

The electrospinning process can be manipulated to meet the specific requirements for any given application of the electrospun material. In one embodiment, a syringe can be mounted on a frame that moves in the x, y, and z planes with respect to the grounded substrate. In another embodiment, a syringe can be mounted around a grounded substrate, for instance, a tubular mandrel. In this way, the materials that form the matrix streamed from the syringe can be specifically aimed or patterned. Although the micropipette can be moved manually, the frame onto which the syringe is mounted can also be controlled by a microprocessor and a motor that allows the pattern of streaming to be predetermined. Such microprocessors and motors are known to one of ordinary skill in the art, for example matrix fibers can be oriented in a specific direction, they can be layered, or they can be programmed to be completely random and not oriented.

The degree of branching can be varied by many factors including, but not limited to, voltage (for example ranging from about 0 to 30,000 volts, such as from 14-16 kV), distance from a syringe tip to the substrate (for example, from 1-100 cm, 0-40 cm, 10-15 cm, or 1-10 cm), the speed of rotation, the relative position of a syringe tip and target (i.e., in front of, above, below, aside, etc.), and the diameter of a syringe tip (approximately 0-2 mm), and the concentration and ratios of compounds that form the electrospun material. Other parameters which are important include those affecting evaporation of solvents, such as temperature, pressure, and humidity. Those skilled in the art will recognize that these and other parameters can be varied to form electrospun materials with characteristics that are particularly adapted for specific applications. In some embodiments, the polymers to be electrospun can be present in the solution at any concentration that will allow electrospinning.

The polymers can be dissolved in any solvent that allows delivery of the polymers to the orifice or tip of a syringe, under conditions that the polymers are electrospun. Solvents useful for dissolving or suspending a material or a substance will depend on the identity of the polymers. Electrospinning techniques often require more specific solvent conditions. In some embodiments, the solvents can be water, 2,2,2-trifluoroethanol, 1,1,1,3,3,3-hexafluoro-2-propanol (also known as hexafluoroisopropanol or HFIP), isopropanol, other lower-order alcohols, halogenated alcohols, acetamide, N-methylformamide, N,N-dimethylformamide (DMF), methylene chloride (DCM), dimethylsulfoxide (DMSO), dimethylacetamide, N-methyl pyrrolidone (NMP), formic acid, acetic acid, trifluoroacetic acid, ethyl acetate, acetonitrile, trifluoroacetic anhydride, 1,1,1-trifluoroacetone, maleic acid, hexafluoroacetone, methanol, chloroform, trifluoroethanol (TFE) or combinations thereof. In some embodiments, the solvents can include formic acid, acetic acid, hexafluoroisopropanol, methylene chloride, N,N-dimethylformamide, trifluoroethanol, or any combination thereof.

The selection of a solvent is based in part on the consideration of secondary forces that stabilize polymer-polymer interactions and the solvent's ability to replace these with strong polymer-solvent interactions. In the case of polypeptides such as collagen, and in the absence of covalent crosslinking, the principal secondary forces between chains are: (1) coulombic, resulting from attraction of fixed charges on the backbone and dictated by the primary structure (e.g., lysine and arginine residues will be positively charged at physiological pH, while aspartic or glutamic acid residues will be negatively charged); (2) dipole-dipole, resulting from interactions of permanent dipoles; the hydrogen bond, commonly found in polypeptides, is the strongest of such interactions; and (3) hydrophobic interactions, resulting from association of non-polar regions of the polypeptide due to a low tendency of non-polar species to interact favorably with polar water molecules. Therefore, solvents or solvent combinations that can favorably compete for these interactions can dissolve or disperse polypeptides.

### Composite Yarn

Described herein are also composite yarns including: a yarn core including one or more polymers, and carbon nanomaterial on the surface of the yarn core. In some embodiments, the composite yarns can be used to form a textile layer of a scaffold for tissue engineering. In some embodiments, the composite yarns can be formed of interlocking bundles of fibers, including the one or more polymers and the carbon nanomaterial. In some embodiments, the yarn core can further include an active agent.

In some embodiments, the yarn core can include one or more polymers selected from, but is not limited to, biodegradable synthetic polymers (e.g., a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), a polyethylene glycol (PEG), polyurethane (PU), a poly(phosphazine), a poly(phosphate ester) or copolymers thereof, and blends thereof), a biopolymer (e.g., a gelatin, a collagen, alginate, chitosan, agarose, fibrin, hyaluronic acid, elastin, silk fibroin, or copolymers thereof, and blends thereof), or copolymers thereof, and blends thereof.

In some embodiments, the one or more polymers can include a biodegradable synthetic polymer, a biopolymer, or any combination thereof. In some embodiments, the one or more polymers can include a biodegradable synthetic polymer. In some embodiments, the one or more polymers can include a biopolymer. In some embodiments, the one or more polymers can include a biodegradable synthetic polymer and a biopolymer. In some embodiments, the yarn core can include a biodegradable synthetic polymer, a biopolymer, or any combination thereof. In some embodiments, the yarn core can include a biodegradable synthetic polymer. In some embodiments, the yarn core can include a biopolymer. In some embodiments, the yarn core can include a biodegradable synthetic polymer and a biopolymer.

In some embodiments, the yarn core can include a biodegradable synthetic polymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the yarn core can include a biodegradable synthetic polymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The yarn core can include a biodegradable synthetic polymer in an amount ranging from any of the minimum values described above to any of the maximum values described above. For example, in some embodiments, the yarn core can include a biodegradable synthetic polymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the yarn core yarn core can include a biopolymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%).

In some embodiments, the yarn core can include a biopolymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The fiber can include a biopolymer in an amount ranging from any of the minimum values described above to any of the maximum values described above. For example, in some embodiments, the yarn core can include a biopolymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the yarn core can include a biodegradable synthetic polymer from greater than 0% to 100% by weight and a biopolymer from 0% to 100% by weight (e.g., a biodegradable synthetic polymer from 0% to 100% by weight and a biopolymer from greater than 0% to 100% by weight, a biodegradable synthetic polymer from 0% to 10% by weight and a biopolymer from greater than 0% to 90% by weight, a biodegradable synthetic polymer from 0% to 20% by weight and a biopolymer from greater than 0% to 80% by weight, a biodegradable synthetic polymer from 0% to 30% by weight and a biopolymer from greater than 0% to 70% by weight, a biodegradable synthetic polymer from 0% to 40% by weight and a biopolymer from greater than 0% to 60% by weight, a biodegradable synthetic polymer fro 0% to 50% by weight and a biopolymer from greater than 0% to 50% by weight, a biodegradable synthetic polymer from 0% to 60% by weight and a biopolymer from greater than 0% to 40% by weight, a biodegradable synthetic polymer from 0% to 70% by weight and a biopolymer from greater than 0% to 30% by weight, a biodegradable synthetic polymer from 0% to 80% by weight and a biopolymer from greater than 0% to 20% by weight, a biodegradable synthetic polymer from 0% to 90% by weight and a biopolymer from greater than 0% to 10% by weight, a biodegradable synthetic polymer from greater than 0% to 100% by weight and a biopolymer 0%-100% by weight).

In some embodiments, the one or more polymers can include polycaprolactone, gelatin, or any combination thereof. In some embodiments, the one or more polymers can include polycaprolactone and gelatin. In some embodiments, the one or more polymers can include polycaprolactone. In some embodiments, the one or more polymers can include gelatin.

In some embodiments, the carbon nanomaterial can include, but is not limited to, carbon nanotubes, graphene, carbon dots, or any combination thereof. In some embodiments, the carbon nanomaterial can include carbon nanotubes. In some embodiments, the carbon nanomaterial can include graphene. In some embodiments, the carbon nanomaterial can include carbon dots. In some embodiments, the composite yarn can further include a coating that promotes cell adhesion. In some embodiments, the coating can include a biomaterial. In some embodiments, the biomaterial can be a hydrogel. In some embodiments, the hydrogel can further include an active agent. In some embodiments, the biomaterial can include extracellular matrix components. In some embodiments, the biomaterial can include, but is not limited to gelatin, alginate, chitosan, agarose, fibrin, collagen, hyaluronic acid, copolymers thereof, and blends thereof. In some embodiments, the biomaterial can include gelatin.

In some embodiments, when the composite yarn includes a coating including a biomaterial, the composite yarn can have a mechanical property selected from a crystalline degree between 0.2 and 0.4 (e.g., between 0.25 and 0.35), a maximum stress of between 1 MPa and 13 MPa (e.g., between 2 MPa and 13 MPa, between 1 MPa and 3 MPa, or between 3.5 MPa and 12 MPa), a maximum strain of between 0.05 and 5 (e.g., between 2 and 5, between 0.05 and 3.5, or between 0.15 and 2.5), an elastic modulus of between 1 MPa and 22 MPa (e.g., between 2 MPa and 19 MPa), and a resilience of between 0.05 MJ/m³ and 12 MJ/m³ (e.g., between 0.2 MJ/m³ and 9.5 MJ/m³), or any combination thereof.

In some embodiments, the scaffold can have a longitudinal-maximum stress of between 1 MPa and 3 MPa such as between 1.2 MPa and 1.7 MPa. In some embodiments, the scaffold can have a longitudinal-maximum strain of between 2 and 5, such as between 2 and 3, between 2 and 4. In some embodiments, the scaffold can have a circumferential-maximum stress of between 1 MPa and 3 MPa such as between 1.2 MPa and 1.7 MPa. In some embodiments, the scaffold can have a circumferential-maximum strain of between 2 and 5, such as between 3 and 5, between 4 and 5.

### Methods of Making Composite Yarn

Described herein are also methods of fabricating composite yarns, the method can include: forming fibers of one or more polymers in a carbon nanomaterial bath, the carbon nanomaterial bath can include a carbon nanomaterial suspended in a liquid; coating the fibers with the carbon nanomaterial to form fibers; extracting the fibers from the carbon nanomaterial bath; and interlocking bundles of fibers to form composite yarns, the composite yarns can include the one or more polymers and carbon nanomaterial. In some embodiments, the fibers can further include an active agent.

In some embodiments, the method can further include coating the composite yarns with a biomaterial that promotes cell adhesion. In some embodiments, the biomaterial can be a hydrogel. In some embodiments, the hydrogel can further include an active agent. In some embodiments, the biomaterial can include extracellular matrix components. In some embodiments, the biomaterial can include, but is not limited to gelatin, alginate, chitosan, agarose, fibrin, collagen, hyaluronic acid, or copolymers thereof, and blends thereof. In some embodiments, the biomaterial can include gelatin.

In some embodiments, when the composite yarn includes a coating including a biomaterial, the composite yarn can have a mechanical property selected from a crystalline degree between 0.2 and 0.4 (e.g., between 0.25 and 0.35), a maximum stress of between 1 MPa and 13 MPa (e.g., between 2 MPa and 13 MPa, between 1 MPa and 3 MPa, or between 3.5 MPa and 12 MPa), a maximum strain of between 0.05 and 5 (e.g., between 2 and 5, between 0.05 and 3.5, or between 0.15 and 2.5), an elastic modulus of between 1 MPa and 22 MPa (e.g., between 2 MPa and 19 MPa), and a resilience of between 0.05 MJ/m³ and 12 MJ/m³ (e.g., between 0.2 MJ/m³ and 9.5 MJ/m³), or any combination thereof.

In some embodiments, the scaffold can have a longitudinal maximum stress of between 1 MPa and 3 MPa such as between 1.2 MPa and 1.7 MPa. In some embodiments, the scaffold can have a longitudinal maximum strain of between 2 and 5, such as between 2 and 3, between 2 and 4. In some embodiments, the scaffold can have a circumferential maximum stress of between 1 MPa and 3 MPa such as between 1.2 MPa and 1.7 MPa. In some embodiments, the scaffold can have a circumferential maximum strain of between 2 and 5, such as between 3 and 5, between 4 and 5.

In some embodiments, the yarn core can include one or more polymers selected from, but is not limited to, a biodegradable synthetic polymers (e.g., a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), a polyethylene glycol (PEG), polyurethane (PU), a poly(phosphazine), a poly(phosphate ester) or copolymers thereof, and blends thereof), a biopolymer (e.g., a gelatin, a collagen, alginate, chitosan, agarose, fibrin, hyaluronic acid, elastin, silk fibroin, or copolymers thereof, and blends thereof), or copolymers thereof, and blends thereof.

In some embodiments, the one or more polymers can include a biodegradable synthetic polymer, a biopolymer, or any combination thereof. In some embodiments, the one or more polymers can include a biodegradable synthetic polymer. In some embodiments, the one or more polymers can include a biopolymer. In some embodiments, the one or more polymers can include a biodegradable synthetic polymer and a biopolymer. In some embodiments, the yarn core can include a biodegradable synthetic polymer, a biopolymer, or any combination thereof. In some embodiments, the yarn core can include a biodegradable synthetic polymer. In some embodiments, the yarn core can include a biopolymer. In some embodiments, the yarn core can include a biodegradable synthetic polymer and a biopolymer.

In some embodiments, the yarn core can include a biodegradable synthetic polymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the yarn core can include a biodegradable synthetic polymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The yarn core can include a biodegradable synthetic polymer in an amount ranging from any of the minimum values described above to any of the maximum values described above. For example, in some embodiments, the yarn core can include a biodegradable synthetic polymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the yarn core yarn core can include a biopolymer in an amount of at least greater than 0% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%). In some embodiments, the yarn core can include a biopolymer in an amount of 100% or less (e.g., 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less).

The fiber can include a biopolymer in an amount ranging from any of the minimum values described above to any of the maximum values described above. For example, in some embodiments, the yarn core can include a biopolymer in an amount of from greater than 0% to 100% by weight (e.g., from greater than 0% to 95%, from greater than 0% to 90%, from greater than 0% to 80%, from greater than 0% to 70%, from greater than 0% to 60%, from greater than 0% to 50%, from greater than 0% to 40%, from greater than 0% to 30%, from greater than 0% to 20%, from greater than 0% to 10%, from greater than 0% to 5%, from 5% to 100%, from 5% to 95%, from 5% to 90%, from 5% to 80%, from 5% to 70%, from 5% to 60%, from 5% to 50%, from 5% to 40%, from 5% to 30%, from 5% to 20%, from 5% to 10%, from 10% to 100%, from 10% to 95%, from 10% to 90%, from 10% to 80%, from 10% to 70%, from 10% to 60%, from 10% to 50%, from 10% to 40%, from 10% to 30%, from 10% to 20%, from 20% to 100%, from 20% to 95%, from 20% to 90%, from 20% to 80%, from 20% to 70%, from 20% to 60%, from 20% to 50%, from 20% to 40%, from 20% to 30%, from 30% to 100%, from 30% to 95%, from 30% to 90%, from 30% to 80%, from 30% to 70%, from 30% to 60%, from 30% to 50%, from 30% to 40%, from 40% to 100%, from 40% to 95%, from 40% to 90%, from 40% to 80%, from 40% to 70%, from 40% to 60%, from 40% to 50%, from 50% to 100%, from 50% to 95%, from 50% to 90%, from 50% to 80%, from 50% to 70%, from 50% to 60%, from 60% to 100%, from 60% to 95%, from 60% to 90%, from 60% to 80%, from 60% to 70%, from 70% to 100%, from 70% to 95%, from 70% to 90%, from 70% to 80%, from 80% to 90%, from 80% to 95%, from 80% to 100%, from 90% to 95%, from 90% to 100%, or from 95% to 100%).

In some embodiments, the yarn core can include a biodegradable synthetic polymer from greater than 0% to 100% by weight and a biopolymer from 0% to 100% by weight (e.g., a biodegradable synthetic polymer from 0% to 100% by weight and a biopolymer from greater than 0% to 100% by weight, a biodegradable synthetic polymer from 0% to 10% by weight and a biopolymer from greater than 0% to 90% by weight, a biodegradable synthetic polymer from 0% to 20% by weight and a biopolymer from greater than 0% to 80% by weight, a biodegradable synthetic polymer from 0% to 30% by weight and a biopolymer from greater than 0% to 70% by weight, a biodegradable synthetic polymer from 0% to 40% by weight and a biopolymer from greater than 0% to 60% by weight, a biodegradable synthetic polymer fro 0% to 50% by weight and a biopolymer from greater than 0% to 50% by weight, a biodegradable synthetic polymer from 0% to 60% by weight and a biopolymer from greater than 0% to 40% by weight, a biodegradable synthetic polymer from 0% to 70% by weight and a biopolymer from greater than 0% to 30% by weight, a biodegradable synthetic polymer from 0% to 80% by weight and a biopolymer from greater than 0% to 20% by weight, a biodegradable synthetic polymer from 0% to 90% by weight and a biopolymer from greater than 0% to 10% by weight, a biodegradable synthetic polymer from greater than 0% to 100% by weight and a biopolymer 0%-100% by weight).

In some embodiments, the one or more polymers can include polycaprolactone. In some embodiments, the one or more polymers can include gelatin. In some embodiments, the one or more polymers can include polycaprolactone and gelatin. In some embodiments, forming yarns of one or more polymers can include electrospinning, wet electrospinning, or any combination thereof.

In some embodiments, the carbon nanomaterial bath has a carbon nanomaterial concentration to provide a composite yarn having at least one of a pre-defined mechanical property, a pre-defined thermal property, or a pre-defined degradation rate range. In some embodiments, the pre-defined mechanical property can be selected from a pre-defined average crystalline degree, pre-defined resilience or range, a pre-defined elastic modulus or range, a pre-defined maximum strain or range, and a pre-defined maximum stress or range, or any combination thereof. In some embodiments, the solvent concentration can be varied to generate the plurality of fibers having a pre-defined average crystalline degree.

Suitable carbon nanomaterial bath liquid can include but is not limited to water, culture media, alcohol (e.g., ethanol, propanol, methanol, butanol, pentanol, hexanol, or any combination thereof), or any combination thereof. In some embodiments, the liquid can include an alcohol. In some embodiments, the liquid can include water. In some embodiments, the liquid can include culture media. In some embodiments, the liquid can be ethanol. In some embodiments, the liquid can be propanol. In some embodiments, the liquid can include water and an alcohol. In some embodiments, the carbon nanomaterial bath liquid can include an ethanol in an amount of greater than 40% (e.g., greater than 50%, greater than 60%, greater than 70%, greater than 80%, or greater than 90%). In some embodiments, the carbon nanomaterial bath liquid can include an ethanol in an amount of 100% or less (e.g., 90% or less, 80% or less, 70% or less, 60% or less, or 50% or less).

The carbon nanomaterial bath liquid can include an ethanol in an amount ranging from any of the minimum values described above to any of the maximum values described above. For example, in some embodiments, the carbon nanomaterial bath liquid can include an ethanol in an amount of from 40% to 100% (e.g., from 40% to 90%, from 40% to 80%, from 40% to 70%, from 40% to 60%, from 40% to 50%, from 50% to 100%, from 50% to 90%, from 50% to 80%, from 50% to 70%, from 50% to 60%, from 60% to 100%, from 60% to 90%, from 60% to 80%, from 60% to 70%, from 70% to 100%, from 70% to 90%, from 70% to 80%, from 80% to 100%, from 80% to 90%, or from 90% to 100%).

In some embodiments, the carbon nanomaterial can include, but is not limited to, carbon nanotubes, graphene, carbon dots, or any combination thereof. In some embodiments, the carbon nanomaterial can include carbon nanotubes. In some embodiments, the carbon nanomaterial can include graphene. In some embodiments, the carbon nanomaterial can include carbon dots.

In some embodiments, the carbon nanomaterial in the carbon nanomaterial bath can be present in an amount of at least greater than 0 mg/L (e.g., at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 110 mg/L, at least 120 mg/L, at least 130 mg/L, at least 140 mg/L, at least 150 mg/L, at least 160 mg/L, at least 170 mg/L, at least 180 mg/L, or at least 190 mg/L). In some embodiments, the carbon nanomaterial in the carbon nanomaterial bath can be present in an amount of 200 mg/L or less (e.g., 190 mg/L or less, 180 mg/L or less, 170 mg/L or less, 160 mg/L or less, 150 mg/L or less, 140 mg/L or less, 130 mg/L or less, 120 mg/L or less, 110 mg/L or less, 100 mg/L or less, 90 mg/L or less, 80 mg/L or less, 70 mg/L or less, 60 mg/L or less, 50 mg/L or less, 40 mg/L or less, 30 mg/L or less, 20 mg/L or less, 10 mg/L or less, or 5 mg/L or less).

The carbon nanomaterial in the carbon nanomaterial bath can be present in an amount ranging from any of the minimum values described above to any of the maximum values described above. For example, in some embodiments, the carbon nanomaterial in the carbon nanomaterial bath can be present in an amount of from greater than 0 mg/L to 200 mg/L (e.g., from greater than 0 mg/L to 180 mg/L, from greater than 0 mg/L to 160 mg/L, from greater than 0 mg/L to 140 mg/L, from greater than 0 mg/L to 100 mg/L, from greater than 0 mg/L to 50 mg/L, from greater than 0 mg/L to 25 mg/L, from greater than 0 mg/L to 10 mg/L, from 5 mg/L to 10 mg/L, from 5 mg/L to 25 mg/L, from 5 mg/L to 50 mg/L, from 5 mg/L to 100 mg/L, from 5 mg/L to 150 mg/L, from 5 mg/L to 200 mg/L, from 10 mg/L to 25 mg/L, from 10 mg/L to 50 mg/L, from 10 mg/L to 100 mg/L, from 10 mg/L to 150 mg/L, from 10 mg/L to 200 mg/L, from 25 mg/L to 50 mg/L, from 25 mg/L to 100 mg/L, from 25 mg/L to 150 mg/L, from 25 mg/L to 200 mg/L, from 50 mg/L to 100 mg/L, from 50 mg/L to 150 mg/L, from 50 mg/L to 200 mg/L, from 100 mg/L to 150 mg/L, from 100 mg/L to 200 mg/L, or from 150 mg/L to 200 mg/L).

### Methods of Use

Described herein are also therapeutic methods, the therapeutic methods can include: providing the scaffold described herein; and implanting the scaffold into or onto a subject.

Described herein are also methods of promoting cell adhesion to a tissue-engineered scaffold, the method can include: fabricating yarns according to the method described herein; fabricating a tissue-engineered scaffold according to the methods described herein; and contacting the tissue-engineered scaffold with cells in an environment that promotes cell viability. In some embodiments, the yarns can include composite yarns. In some embodiments, the method can further include promoting cell proliferation on the tissue-engineered scaffold.

In some embodiments, the cells can include, but are not limited to, connective tissue cells, organ cells, muscle cells, nerve cells, tenocytes, fibroblasts, ligament cells, endothelial cells, lung cells, epithelial cells, smooth muscle cells, cardiac muscle cells, skeletal muscle cells, islet cells, nerve cells, hepatocytes, kidney cells, bladder cells, urothelial cells, chondrocytes, bone-forming cells, induced pluripotent stem cells, adipose stem cells, bone marrow stem cells, synovium stem cells, dental pulp stem cells, neural stem cells, mesenchymal stem cells, chondrocytes, osteoblasts, myoblasts, myeloid cells, endothelial progenitor cells, any other cells from which a tissue scaffold may be generated, or any combination thereof. In certain embodiments, the scaffold can be used to produce organ tissue for implantation into an animal, such as, e.g., skin tissue for skin grafts, myocardial tissue, bone tissue for bone regeneration, testicular tissue, or blood vessels. Thus, those of skill in the art would understand that the aforementioned organs/cells are merely exemplary organs/cell types and it should be understood that cells from any organ may be seeded onto the scaffolds described herein to produce useful tissue for implantation and/or study.

The cells that may be seeded onto the scaffolds described may be derived from commercially available cell lines, or alternatively may be primary cells, which can be isolated from a given tissue by disaggregating an appropriate organ or tissue, which is to serve as the source of the cells being grown. This may be readily accomplished using techniques known to those skilled in the art. Such techniques include disaggregation through the use of mechanical forces either alone or in combination with digestive enzymes and/or chelating agents that weaken cell-cell connections between neighboring cells to make it possible to disperse the tissue into a suspension of individual cells without appreciable cell breakage. Enzymatic dissociation can be accomplished by mincing the tissue and treating the minced tissue with any of a number of digestive enzymes, either alone or in combination. Digestive enzymes include but are not limited to trypsin, chymotrypsin, collagenase, elastase, and/or hyaluronidase, Dnase, pronase, etc. Mechanical disruption can also be accomplished by a number of methods including, but not limited to the use of grinders, blenders, sieves, homogenizers, pressure cells, or sonicators, to name but a few. For a review of tissue disaggregation techniques, see Freshney, Culture of Animal Cells. A Manual of Basic Technique, 2d Ed., A. R. Liss, Inc., New York, 1987, Ch. 9, pp. 107-126.

Once the primary cells are disaggregated, the cells are separated into individual cell types using techniques known to those of skill in the art. For a review of clonal selection and cell separation techniques, see Freshney, Culture of Animal Cells. A Manual of Basic Techniques, 2d Ed., A. R. Liss, Inc., New York, 1987, Ch. 11 and 12, pp. 137-168. Media and buffer conditions for the growth of the cells will depend on the type of cell, and such conditions are known to those of skill in the art.

In certain embodiments, the cells attached to the scaffolds described herein are grown in bioreactors. A bioreactor may be of any class, size, or have any one or number of desired features, depending on the product to be achieved. Different types of bioreactors include tank bioreactors, immobilized cell bioreactors, hollow fiber, and membrane bioreactors as well as digesters. There are three classes of immobilized bioreactors, which allow cells to be grown: membrane bioreactors, filter or mesh bioreactors, and carrier particle systems. Membrane bioreactors grow the cells on or behind a permeable membrane, allowing the nutrients to leave the cell, while preventing the cells from escaping. Filter or mesh bioreactors grow the cells on an open mesh of an inert material, allowing the culture medium to flow past, while preventing the cells from escaping. Carrier particle systems grow the cells on something very small, such as small nylon or gelatin beads. The bioreactor can be a fluidized bed or a solid bed. Other types of bioreactors include pond reactors and tower fermentors. Any of these bioreactors may be used for regenerating/engineering tissues on the scaffolds described herein.

In certain embodiments, the cells are genetically engineered cells that have been modified to express a biologically active or therapeutically effective protein product. Techniques for modifying cells to produce the recombinant expression of such protein products are well known to those of skill in the art. In some embodiments, the scaffolds may be used to form of a tissue graft or tissue patch. Such a tissue graft may be an autograft, allograft, biograft, biogenic graft or xenograft. Tissue grafts may be derived from various tissue types. Representative examples of tissues that may be used to prepare biografts include, but are not limited to, rectus sheaths, peritoneum, bladder, pericardium, veins, arteries, diaphragm, and pleura. For such grafts the cells may be endothelial cells, ligament tissue, muscle cells, bone cells, cartilage cells. Such cells may be grafted into the compositions of the invention alone or in combination with a drug or biologically active agent to be delivered to an in vivo site. For example, such cells for the biograft may be harvested from a host, loaded with the agent of interest and then applied in a perivascular manner at the site where lesions and intimal hyperplasia can develop. Once implanted, the agent of interest (e.g., paclitaxel) is released from the graft and can penetrate the vessel wall to prevent the formation of intimal hyperplasia at the treatment site.

In some embodiments, the scaffolds described herein can be used for delivering a specific active agent to the skin, or an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, Eustachian tubes, the external auditory mayal, vas deferens, and fallopian tubes. Examples of cavities include the abdominal cavity, the buccal cavity, the peritoneal cavity, the pericardial cavity, the pelvic cavity, perivisceral cavity, pleural cavity, and uterine cavity.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope, and concept of the disclosure as defined by the appended claims.

By way of non-limiting illustration, examples of certain embodiments of the present disclosure are given below.

### EXAMPLES

### Example Tissue Engineering System

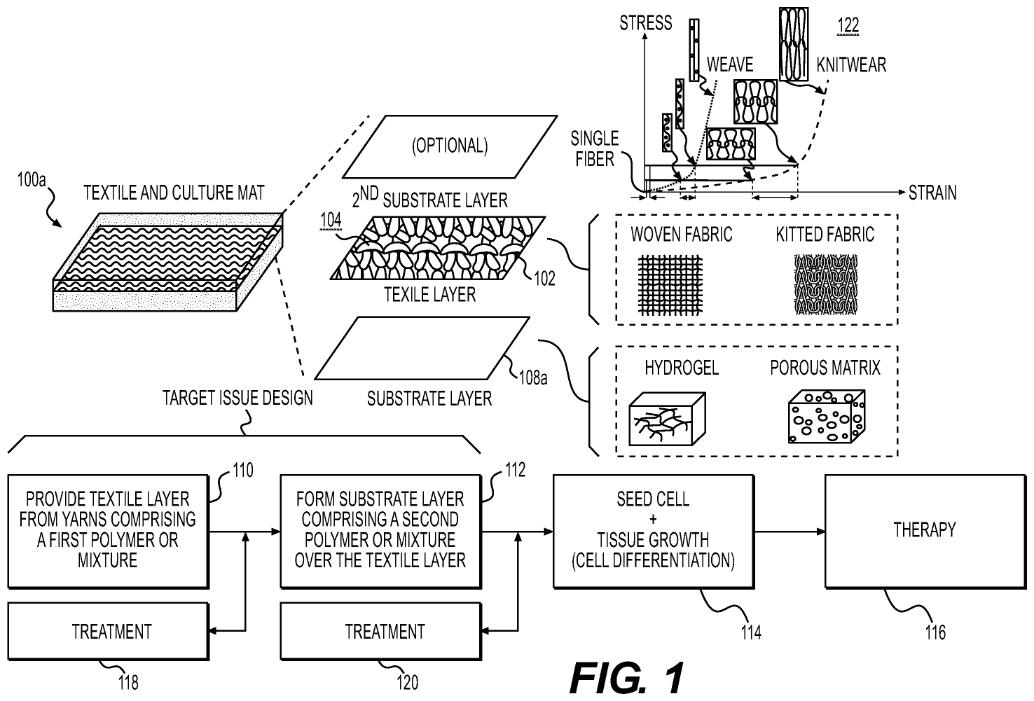

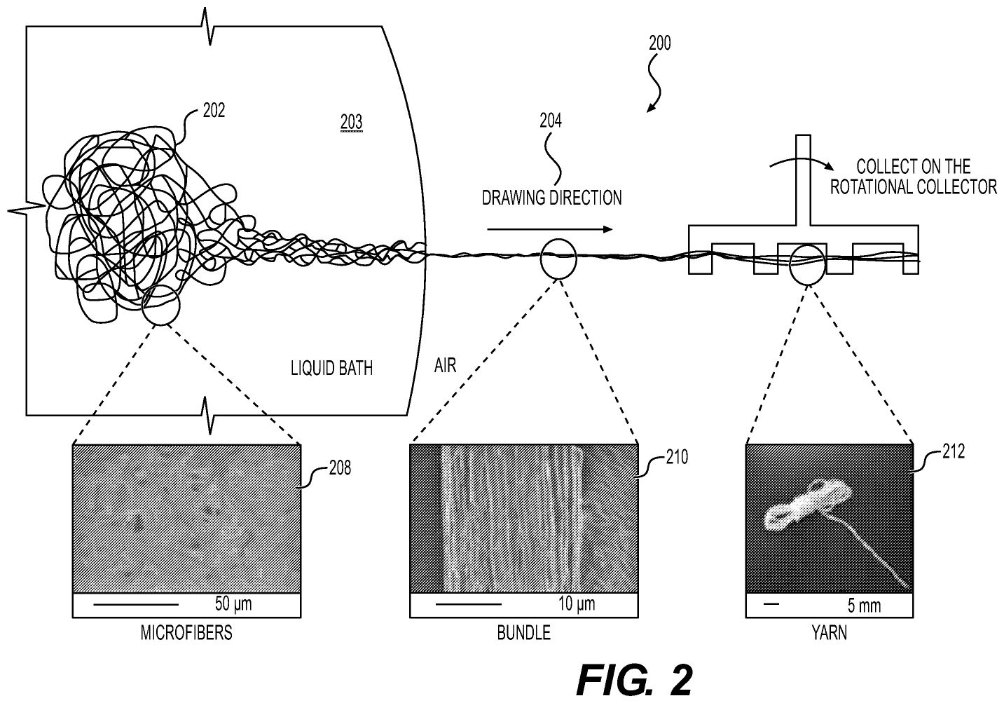

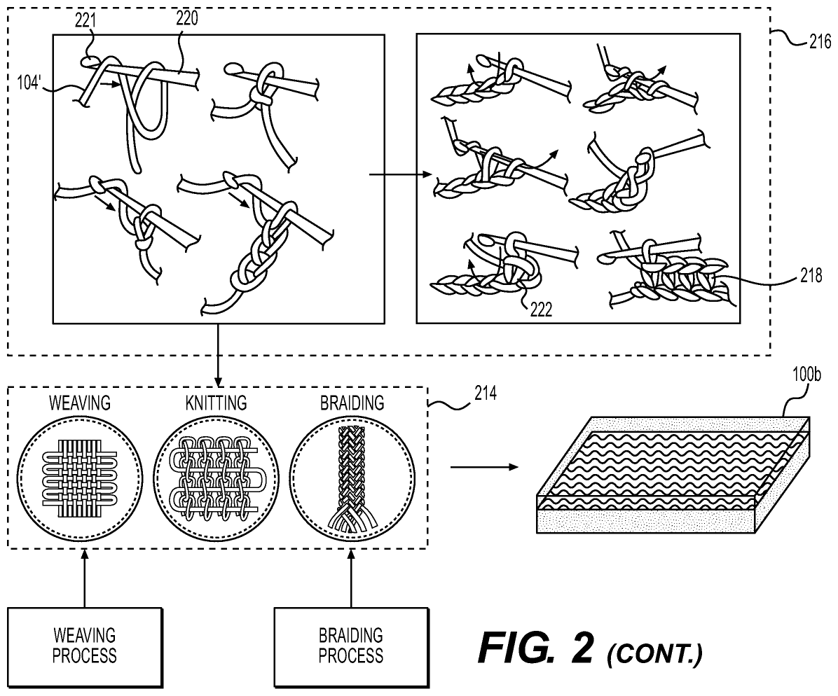

FIG. 1 shows an example tissue engineering system 100 (shown as 100a) comprising a textile layer 102 formed of a plurality of yarns 104 in which the yarns 104 are formed of interlocking bundles of fibers 106 (not shown—see FIG. 2) to provide a scaffold for one or more substrate layers 108. In the example shown in FIG. 1, the substrate layer 108 includes a first substrate layer 108a. The substrate layer 108 may include additional substrate layers (e.g., 108b) in certain embodiments, e.g., as further described herein.

In the example shown in FIG. 1, the textile layer 102 is formed from a synthetic polymer or a polymer mixture that is fabricated using a combination of one more textile fabrication operation such as weaving, knitting, crocheting, knotting, tatting operation, felting operation, bonding operation, or braiding to have a pattern that has a desirable cell or tissue growth, e.g., to form a woven fabric or a knitted fabric.

The textile layer 102 may be fabricated to have a desired or pre-defined nano- and/or the micro-scale as well as macro-scale hierarchical organization for the tissue to have a strain-stiffening property similar to human tissue.

Human skin, for example, has collagen molecules that are winded into a triple helix and then packed into collagen fibers. The collagen fibers then interweave with each other fibers and are embedded in an elastin matrix to form the middle layer of the skin tissue. This hierarchical organization of the skin forms a high sensitivity and high responsiveness material towards stress.

The yarns 104 are configured to have sufficient mechanical strength and be highly biocompatibility. The yarns 104 may be forms of polycaprolactone (PCL) yarns, e.g., by a wet electrospinning process, or other polymer or mixtures such as polyester, polylactic acid (PLA), polyglycolic acid (PGA), polyethylene oxide (PEO), poly lactic-co-glycolide (PLGA), polydioxanone (PDS), polyhydroxyalkanoate (PHA), polyurethane (PU), poly(phosphazine), poly(phosphate ester), gelatin, collagen, alginate, chitosan, agarose, fibrin, hyaluronic acid, polyethylene glycol (PEG), elastin, silk fibroin, copolymers thereof, and blends thereof, and other material described herein.

The substrate layer 108 (also referred to herein as an outer layer) is formed from a polymer or a polymer mixture such as a hydrogel or porous matrix such as a polymer or polymer mixture including polyester, polylactic acid (PLA), polyglycolic acid (PGA), polyethylene oxide (PEO), poly lactic-co-glycolide (PLGA), polycaprolactone (PCL), polydioxanone (PDS), polyhydroxyalkanoate (PHA), polyurethane (PU), poly(phosphazine), poly(phosphate ester), gelatin, collagen, alginate, chitosan, agarose, fibrin, hyaluronic acid, polyethylene glycol (PEG), elastin, silk fibroin, copolymers thereof, and blends thereof, and other material described herein, to which cells can be seeded for cell growth. The thickness of the substrate layer(s) 108 may be optimized, e.g., by controlling the duration of the electrospinning process, to facilitate the initial cell attachment as well as for long-term cell growth. In some embodiments, the cells can be additionally seeded on the textile layer or seeded only at the textile layer 102. In some embodiments, the one or more substrate layer 108 is an electrospun mat.

In combination, the substrate layer 108 and the textile layer 102 as a scaffold of the substrate layer 108 can be configured to have a strain-stiffening behavior and properties such as maximum stress, maximum strain, and/or elastic modulus having a range similar to those of human skin or desired issue. Indeed, the customizable and tailorable textile-based scaffold structure and a tissue engineering tool and method described herein can provide engineered tissue that can more readily mimic the physical, mechanical, and biological properties of human skin and other desired tissues and organs, including having strain-stiffening properties. A plot 122 is shown of stress-stress properties of the textile-based scaffold structure.

To form the example tissue engineering system 100, a method includes providing (110) a scaffold comprising a textile layer 102 from yarns 104 comprising a first polymer or mixture; forming (112) one or more substrate layers 108 comprising a second polymer or mixture over textile layer 102; seedings (114) cells and growing tissue on the textile layer 102 or the substrate layer (108), and using (116) the synthesized tissue in a therapy. The textile layer 102 and substrate layer may be collectively or individually treated (shown as 118, 120) with bioactive molecules such as growth factors, cell adhesion peptides, cytokines, and/or enzymes in vivo to promote tissue growth and cell differentiation.

Examples of cells include induced pluripotent stem cells, adipose stem cells, bone marrow stem cells, synovium stem cells, dental pulp stem cells, neural stem cells, mesenchymal stem cells, chondrocytes, osteoblasts, myoblasts, fibroblasts, myeloid cells, endothelial cells, endothelial progenitor cells, or any combination thereof, as well as other cells described herein.

The engineered tissue (per its use in therapy 116) may be used to form skin tissue, blood vessel tissue, heart valve tissue, cardiac muscle tissue, lung tissue, skeletal muscle tissue, smooth muscle tissue, organ tissue, nerve tissue, connective tissue (e.g., tendon), cartilage, among other tissues described herein, e.g., for grafts or as new organs. To repair or replace the damaged tissues [5], researchers in tissue engineering fields have fabricated artificial tissues which can be implanted into the human body.

The exemplary engineered tissue can generate a 3-dimensional (3D) framework, i.e., having a scaffold, to provide structural integrity for the organism and the micro-environment for the cell growth at the initial stage and then degrade when cells can grow into the new tissue. The exemplary scaffold can have similar physical, biological, and mechanical properties as the target tissue. Among them, the exemplary engineered tissue can mimic tissue's mechanical properties and reduce or eliminate mismatches between the scaffolds and tissues, which can aggravate the damage or, in some cases, lead to cell death in nearby tissue, causing implant failure. The exemplary engineered tissue can have these unique mechanical properties of human tissues employing synthetic materials described herein.

It is noted that the strain-stiffening property of human tissues, which can protect tissues from damage under tensile force, can be difficult to be fabricated and/or replicate by using most biopolymers that have the strain-softening property [6]. Some research has proposed to induce strain-stiffening property by introducing fiber corrugations, such as wavy and wrinkled two-dimensional sheets [7] and helically coiled one-dimensional wires [6], into the structure, which can be fabricated using the fabrication process described herein. Textile technology and the described fabrication process has benefits in mimicking the strain-stiffening property of human tissues to control the fiber corrugation by using various textile methods, such as weaving, knitting, and braiding [8,9].

FIG. 2 shows a method 200 of fabricating a scaffold textile layer 102 in accordance with an illustrative embodiment. In FIG. 2, the method 200 of forming the yarn 104 includes electrospinning (202) a polymer or polymer mixture in a bath solution 203 into a plurality of fibers 106 (shown as 208) then drawing (204) the plurality of fibers out of an electrospinning bath 203 to form a plurality of bundles (210), e.g., by drawings the fibers and winding the drawn fibers around a roller 206 (shown as “collect on the rotational collector” 206). The bundles 210 are then aggregated to form the yarn 104 (shown as 212). Among other benefits, the exemplary method improves upon the whipping phenomenon of electrospinning.

The speed at which the fibers are drawn may be varied to form a desired or pre-defined fiber alignment and a desired or pre-defined bundle diameter. Examples of the polymer or polymer mixture includes polyester, polylactic acid (PLA), polyglycolic acid (PGA), polyethylene oxide (PEO), poly lactic-co-glycolide (PLGA), polycaprolactone (PCL), polydioxanone (PDS), polyhydroxyalkanoate (PHA), polyurethane (PU), poly(phosphazine), poly(phosphate ester), gelatin, collagen, alginate, chitosan, agarose, fibrin, hyaluronic acid, polyethylene glycol (PEG), elastin, silk fibroin, copolymers thereof, and blends thereof.

The scaffolds formed by the textile layer 102 can mimic the anisotropic nature of human tissues, including for nerves [1], tendons [2], blood vessels [3], and the heart [4], among other tissues described herein, by being designed and form with certain textile patterns, including traditional textile patterns, using textile processing techniques such as weaving, knitting, crocheting, knotting, tatting operation, felting operation, bonding operation, or braiding (collectively shown as “Weaving,” “Knitting,” and “Braiding” 214). That is, the textile layer can be fabricated by controlling the fiber orientation, pore geometry, and surface topography of the yarn and/or fibers, so it is suitable for the tissue of interest, such as cartilage, ligament, tendon, nervous, and cardiac tissue. These properties can also control the mechanical properties of scaffolds including strain-stiffening properties and thus the tissue engineering system 100 (shown as 100b). The exemplary textile-based scaffolds can be used as cell-laden fibers to mimic the biological properties of human tissues.

In FIG. 2, an example of crocheting process 216 and pattern 218 is shown. In the example, a hook apparatus 220 having a hook 221 can be used to draw the yarn 104 (shown as 104′) through the prior loop to form a new loop to form a chain of loops. Secondary loops 222 can be formed by drawing the hook through prior loops in the chain. Different hook sizes, 2 mm, 4 mm, 6 mm, among other described herein, may be employed used to fabricate fabrics with different pore sizes. Traditional textile processing machines such as punchcard knitting machines, manual knitting machines, electronic knitting machines, and electronic crochet machines may be used. For finer textile patterns, customed-sized machines based on these traditional processing operations can be built and employed.

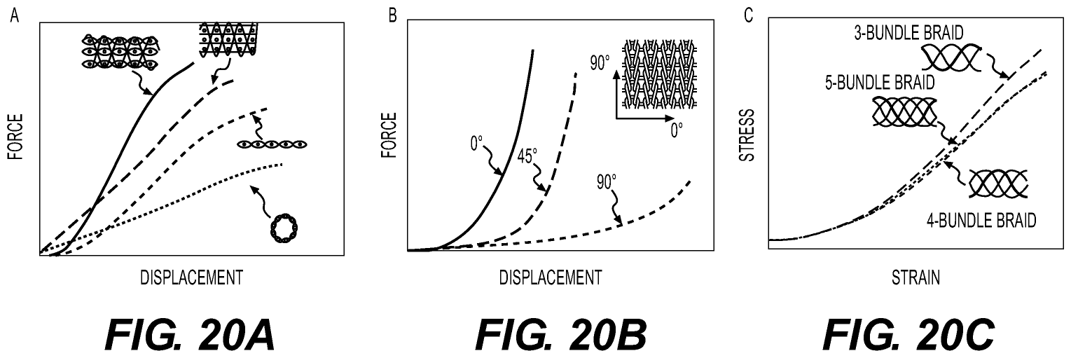

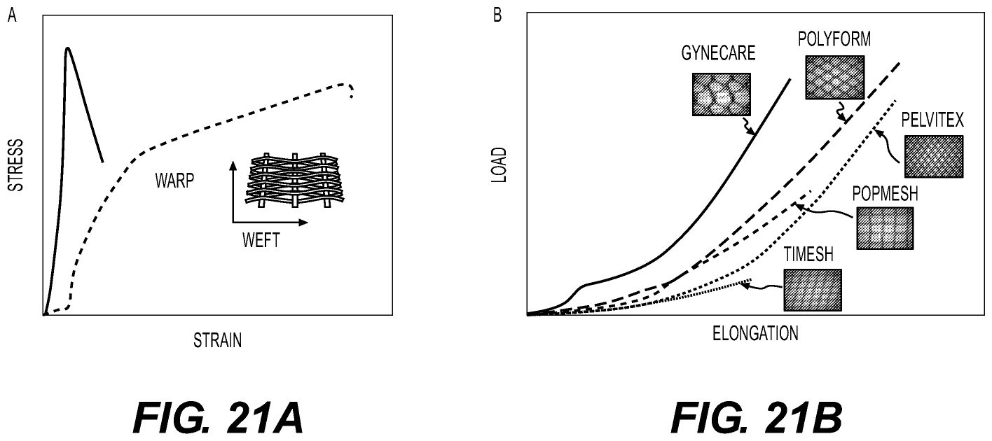

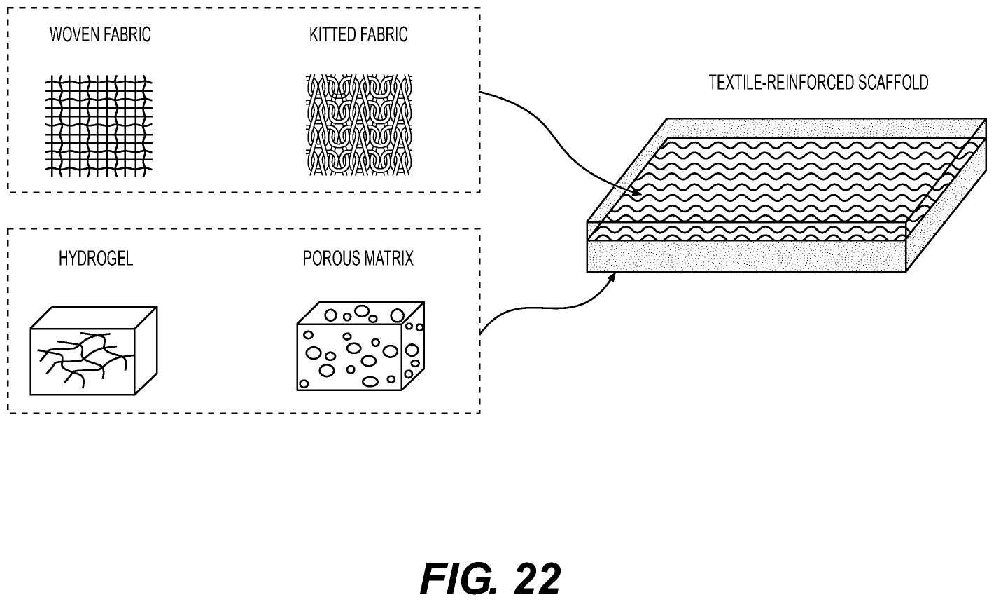

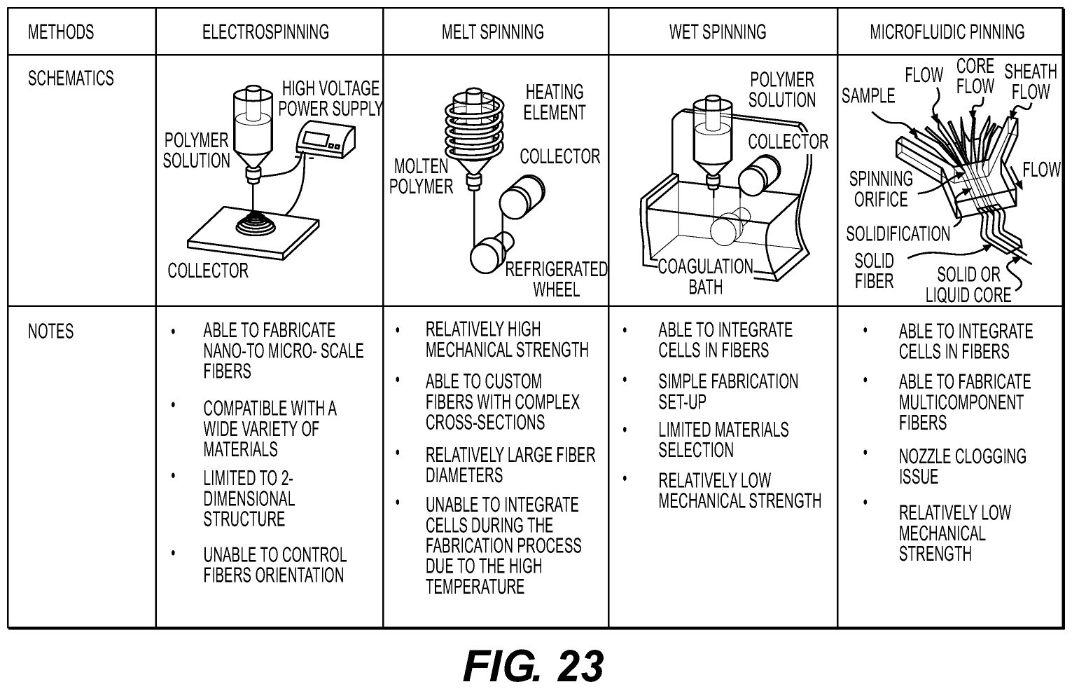

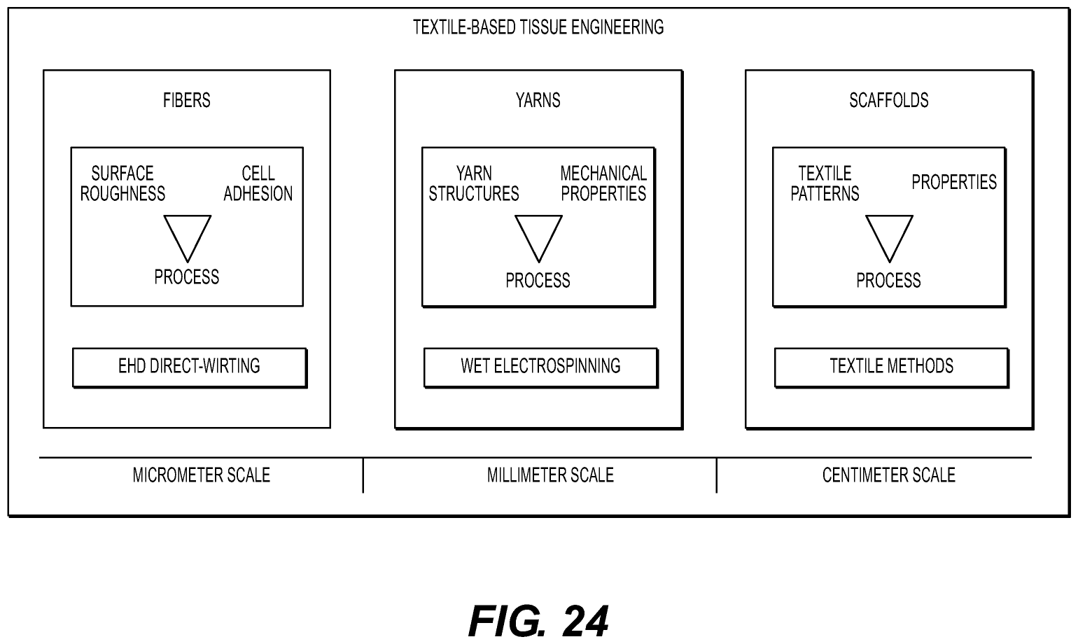

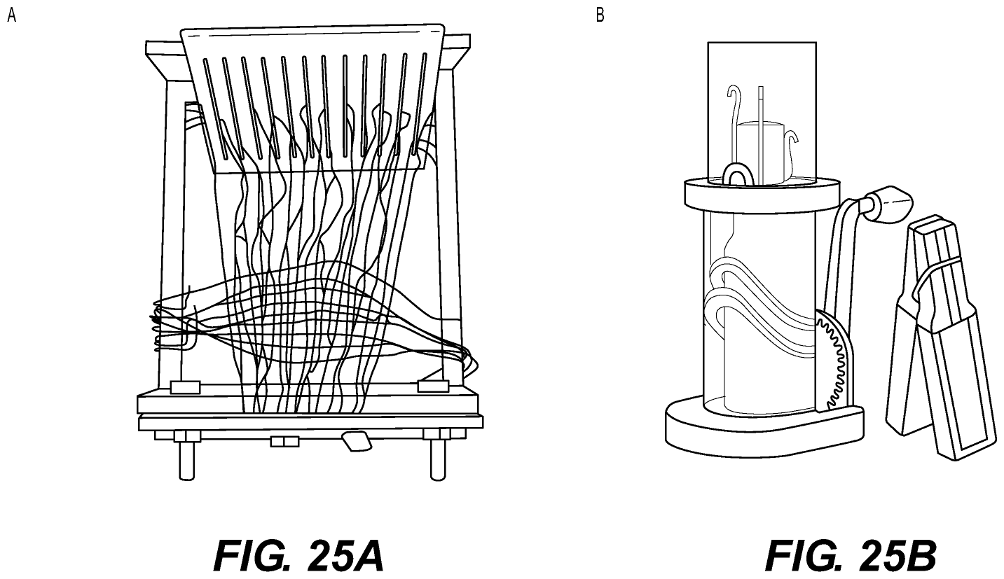

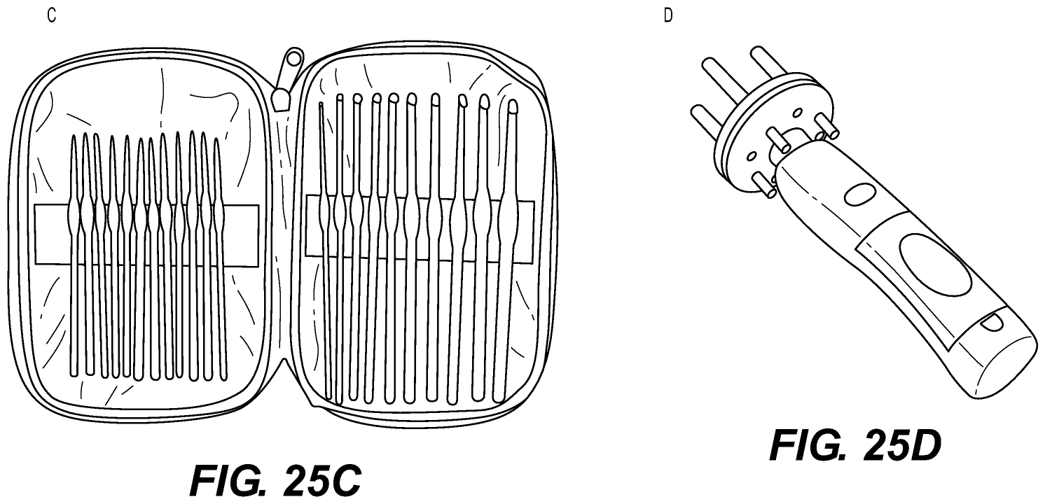

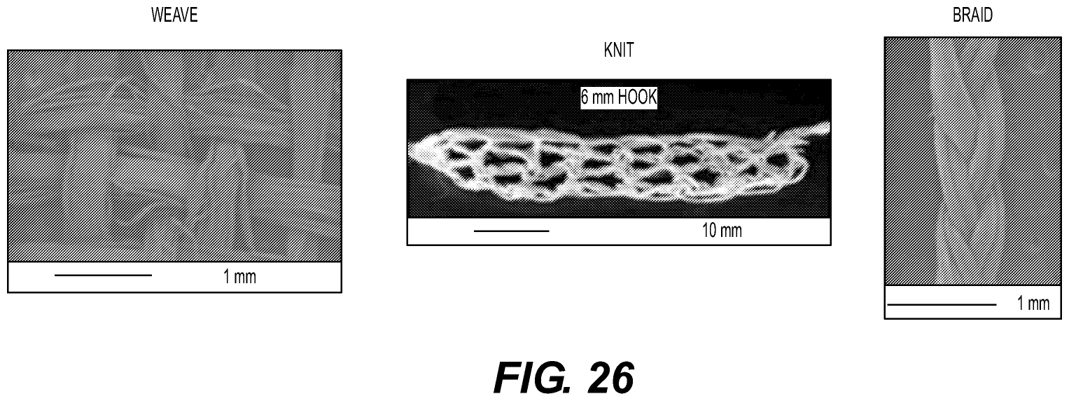

Textile technology has been used to generate wavy fibers and twisting collagen fibers for bone tissue engineering. The woven scaffolds may have wavy structures which share great similarities to the crimps in the tendon [11, 14, 43-45]. The knitted scaffolds exhibit different mechanical properties in different directions to mimic bone's anisotropic property (FIG. 20B) [12, 46]. The braided scaffolds have been observed to have a stable structure without the necessity of edge bonding of woven and knitted scaffolds, and their mechanical properties can be tuned by varying the number of braided bundles (FIG. 20C) [28]. Textile technology has also been used in cardiac tissue engineering, e.g., woven scaffolds have been explored to mimic the anisotropic properties of cardiac tissue using different materials such as the warps and wefts (FIG. 21A) [37, 38, 47]. 3D anisotropic properties have been observed to be achievable by stacking multiple woven fabrics with a gradual transition of alignment [37]. Knitted scaffolds have the potential to mimic the anatomical properties of the cardiac tissues by using various knitted patterns (FIG. 21B) [48]. Also, using conductive yarns in cardiac tissue scaffolds has the potential to simulate the electrophysiology of the cardiac tissues. Textile technology has also been used in skin tissue engineering because of the controllable permeability, scalability, and elastic properties of textile fabrics. Woven [39] and knitted fabrics [40] can be embedded in hydrogel [40-42], porous materials [39], or cell sheets [25] to mimic the three-layer structures of skin tissues (FIG. 22). Most textile-based scaffolds currently use commercial fibers or yarns [15,49,50], making tuning of their properties difficult for different requirements. Fiber fabrication processes and systems have been reported [51]. Among them, electrospinning has been widely used in tissue engineering to fabricate micro- to nano-fibers which have similar physical structures as the extracellular matrix (ECM). Electrospinning can be used with a wide variety of materials selection, e.g., natural polymers, synthetic polymers [52], and has the ability to incorporate bioactive molecules (e.g., growth factors, cell adhesion peptides) in fibers [53] to form electrospun fibers that mimic the mechanical and biological properties of human tissues.

These textile and processing methodologies can be employed with the exemplary yarn fabrication processes described herein.

### Example 2. Yarns with Controllable Mechanical Properties for Textile-Based Tissue Engineering

A method is disclosed to vary the solute-solvent-liquid bath interactions during the wet electrospinning process and the process-structure-property relationship of wet electrospun yarns. The method may be used to fabricate yarns (e.g., 104) with controllable mechanical properties for flexible and rapid textile-based tissue engineering.

Exemplary methods are disclosed herein that can form well-aligned electrospun fibers without the limitation of fiber length well-aligned electrospun fibers using either electrohydrodynamic (EHD) direct-writing or wet electrospinning. Although electrospinning has shown benefits in tissue engineering, the randomness of electrospun fibers makes them difficult to be directly used in textile-based tissue engineering. To address this limitation, various methods have been proposed to modify the conventional electrospinning and achieved great success to generate uniaxially aligned fibers. Modified collectors have been explored, such as using rotational mandrel [54], ring and center point electrodes [55] or parallel electrodes [43], instead of the convention plate collector, but they can limit the length of the well-aligned fibers to physical dimensions of these collectors.

Wet Electrospinning Process. Wet electrospinning can modify conventional electrospinning by replacing the solid collector with the liquid collector. It can be used to lower manufacturing costs and production time for the applications that the post-modification of electrospun fibers is needed. For example, the coagulation bath in wet electrospinning can be used to crosslink the electrospun fibers [71] and load functional nanoparticles on electrospun mats [72,73]. Wet electrospinning has also been used to increase the pore size and thickness of electrospun scaffolds [74-76]. For example, the pore size of electrospun silk fibroin scaffolds was increased from 1.3-2.4 mm in conventional electrospinning to 586-931 mm in wet electrospinning [75].

Wet electrospinning can also be used to fabricate well-aligned fibrous yarns by adding a rotational mandrel to extract electrospun fibers from the liquid bath, which has gained the attention of researchers in tissue engineering. Myung-Seob Khil et al. used wet electrospun yarns to fabricate three-dimensional (3D) scaffolds via plain weaving [79]. MCF-7 mammary carcinoma cells seeded in the woven scaffolds proliferated into the microstructure, suggesting wet electrospun yarns are suitable candidates as scaffolds building blocks. Ling Wang et al. used the wet electrospun yarns as the core and the hydrogel as the shell to mimic the connective tissues [80]. C2C12 myoblasts seeded within the core-shell scaffolds aligned in the yarn direction, and 3D elongated myotubes were observed after long-term cultivation.

The mechanism of wet electrospinning has been investigated to make full use of this technology in textile-based tissue engineering in the exemplary processes described herein, including for a water/ethanol mixture. The solute-solvent-liquid bath interaction in the exemplary wet electrospinning process provides a platform to tune the yarn's properties.

Although wet electrospun yarns have shown advantages in tissue engineering, it had been limited with respect to available materials selection. And, while the wet electrospinning mechanism has not been previously fully understood, which impedes the capability to produce wet electrospun yarns with controllable properties, it has been previously shown that the yarns' morphology, such as the alignment of fibers in the yarn, could be affected by the bath type [78, 106, 107]. Unlike conventional electrospinning, which is applicable to a wide variety of materials, the wet electrospinning process has several requirements for the selection of the solute-solvent-liquid bath system. The materials used as the solute should have sufficient mechanical properties to stand the drawing process and are unable to dissolve in the liquid bath. Currently, the most widely used solute used is PCL because of its good mechanical properties [79]. Other materials such as chitosan [80], silk fibroin (SF) [37], carbon nanotubes (CNT) [80], and polyaniline (PANI) [37] have been investigated to add in PCL to enhance its biological performance.

Materials and experimental setup. PCL (Mₙ=80,000, product number: 440744), DCM (product number: 270997), DMF (product number: 227056) and GA (product number: G6257) were purchased from Sigma-Aldrich®. The exemplary study prepared PCL solution in the scaffold fabrication process by dissolving PCL in the DCM: DMF (V/V ratio: 2:1) cosolvent at a weight concentration of 10%. The prepared PCL solution was then loaded into a syringe which was connected to a syringe pump (LEGATO 100, KD Scientific Co., USA). High voltage (PS/FJ30R04.0, Glassman High Voltage Co., USA) was applied at the nozzle (21G).

Fabrication process of microfibers→bundle→yarn. The exemplary study used a culture dish which was covered by the grounded aluminous foil at the bottom as the liquid container. The take-up roller was controlled by a stepping motor (Silverpak 17C, LIN ENGINEERING, USA). The exemplary study carried out the wet electrospinning process in the ambient environment (Error! Reference source not found.A). The applied voltage was set as 10 kV, the flow rate was set as 40 μl/min, and the distance between the nozzle tip to the collector's bottom was set as 40 mm. The exemplary study first deposited the randomly looped microfibers in the liquid bath (Error! Reference source not found.6B), and then drawn out of the liquid bath to form the bundle. After collecting bundles on the take-up roller for 20 min, a yarn was formed. Black tape was added at the bottom of the collector for observing the deposited microfibers in the liquid bath by a super-speed camera (SMM-C012-U, Mightex Co., USA). The exemplary study evaluated the morphologies of the microfibers and bundles via a scanning electron microscope (SEM, SU8010, Hitachi, Japan) at an accelerating voltage of 3 kV accelerating voltage after gold sputtering.

Textile process. After fabricating the yarn, the exemplary study fabricated the textile fabrics using a crocheting process, which used a hook to consistently draw the yarn through the old loop to form a new loop (Error! Reference source not found.7A-7B). Different hook sizes, 0.25 mm, 0.5 mm, 0.75 mm, 1 mm, 1.25 mm, 1.5 mm, 1.75 mm, 2 mm, 2.25 mm, 2.5 mm, 2.75 mm, 3 mm, 3.25, mm, 3.5 mm, 3.75 mm, 4 mm, 4.25 mm, 4.5 mm, 4.75 mm, 5 mm, or greater may be used to fabricate fabrics with different pore sizes. The single-chain (Error! Reference source not found.7A) could be extended to multiple chains (Error! Reference source not found.7B) by changing the crocheting direction.

Mechanical testing. Before the mechanical testing, the sample was fixed in an aperture card according to ASTM C1557[108]. The tensile tests were performed in a universal testing machine (ESM303, MARK-10, USA) at a rate of 20 mm/s until failure.

Statistics. All experiments were performed with five replicates for each sample. Quantitative data of microfiber alignment were obtained using Image J software (NIH, USA). The relations between microfiber alignment and drawing speed were presented as mean standard deviation.

### Results

The effect of drawing speed on wet electrospinning. The SEM images in Error! Reference source not found.A-17H show that each bundle comprised multiple microfibers. The bundle diameter and the microfiber's orientation could be regulated by the drawing speed (Error! Reference source not found.8A-8B). At low drawing speed, increasing the drawing speed facilitated the alignment of microfibers in a bundle, which makes the bundle diameter more uniform. At high drawing speed, almost all microfibers in a bundle aligned well, so increasing the drawing speed didn't significantly affect the alignment degree of microfibers. However, increasing the drawing speed was observed to increase the variance of the bundle diameter as some microfibers deformed or broke at high drawing speed. The study employed a moderate drawing speed, 0.6-1 m/s.

The effect of liquid bath on wet electrospinning. The exemplary study used various ethanol/water concentrations as the liquid bath. It was found that bundles easily broke off during the drawing process when the ethanol/water ratio was lower than 0.5. Deposited microfibers exhibited the thread-like morphology “diving” in the alcohol bath (Error! Reference source not found.A) while exhibiting the web morphology “floating” in the water bath (Error! Reference source not found.B). The surface tension of the liquid bath may have an effect. It was observed that the higher surface tension of water (72.8 mN/m) makes it more difficult for electrospun microfibers to “dive” into the bath and leads to a larger dragging force on the extracted bundle than does the lower surface tension of alcohol (22.1 mN/m). The surface tension of the liquid bath was observed to decrease as the alcohol concentration increased. In the exemplary study, the PCL bundle was observed to stand the dragging force when the alcohol concentration was larger than 0.5 (surface tension is lower than 28.52 mN/m)[109].

Mechanical properties. The tensile testing results (Error! Reference source not found.12A-12C) showed that the textile fabrics, especially the multiple chains, exhibited an S-shape tensile curve, although the component, yarn, didn't exhibit such behavior. The initial stress in the single chain and multiple chains can be used to realign the wavy fibers in the textiles, resulting in the toe region in the stress-strain curve. This result confirms that textile technology can be used to induce the strain-stiffening property and also demonstrates that wet electrospun yarns can be used in the textile process, and the textile fabrics can mimic the strain-stiffening property of human tissues.

### Example 3. Scaffolds with Designable Textile Patterns to Mimic the Physical, Biological, and Mechanical Properties of Human Tissues

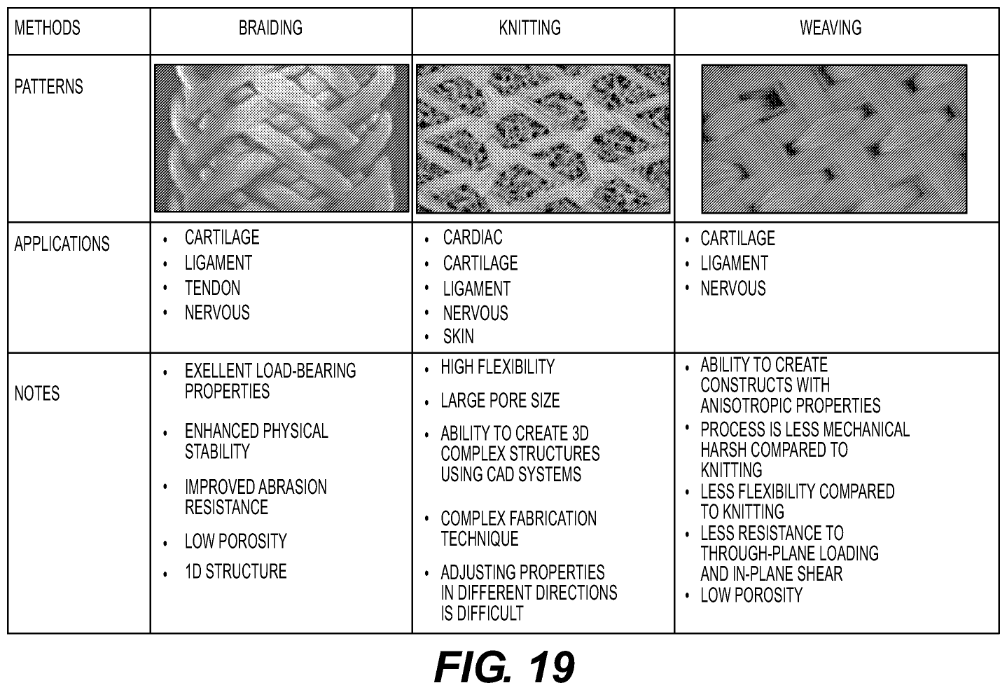

A method is also disclosed to assemble wet electrospun yarns into scaffolds via the textile technology that mimics the physical, biological, and mechanical properties of human tissues. A database of yarns is disclosed that facilitates the customization of yarns made of synthetic materials for the textile-based tissue engineering and related fields. FIG. 19 shows examples of textile patterns of the textile layer and corresponding tissue that can be engineered or grown on such tissue engineering systems 100a.

In some embodiments, wet electrospun yarns are formed into textile fabrics via crocheting, a kind of knitting methods, and test these fabrics' mechanical properties. A database of yarns by using different materials as the solute, solvent, and liquid bath can be generated for the customization of yarns for flexible and rapid textile-based tissue engineering. Textile patterns by using yarns, e.g., developed in Example 2, as a designed textile-based scaffold can be used to mimic the physical, biological, and mechanical properties of human tissues. In an example, the textile fabric scaffolds are encapsulated within one or more electrospun mats. The skin, as the most voluminous organ of the human body, is a good representative of human soft tissues from both componential and mechanical aspects.

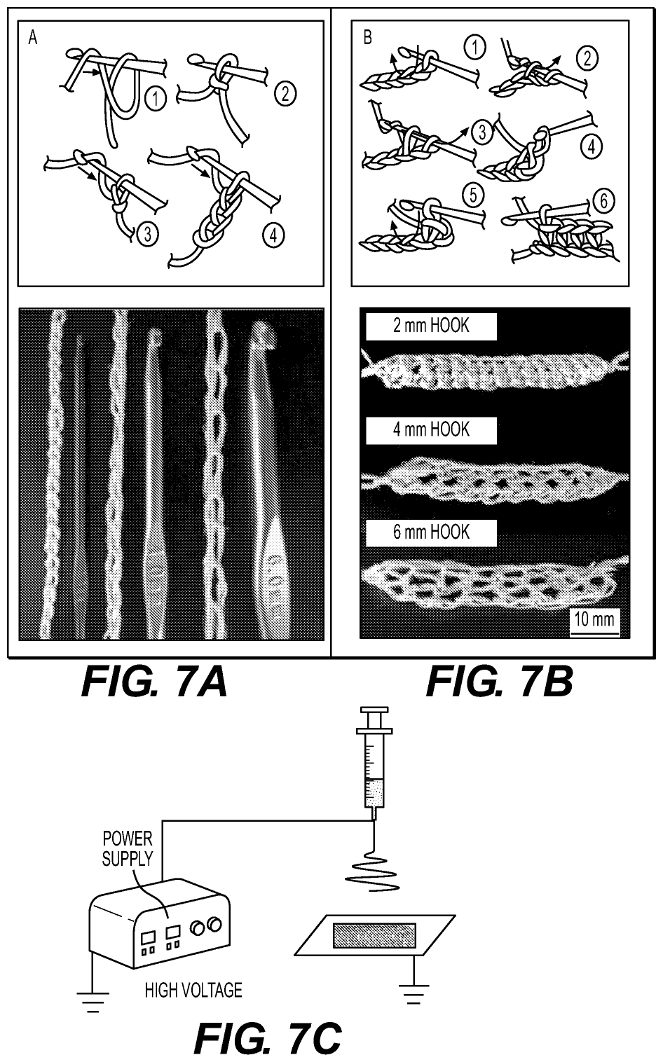

The textile patterns (e.g., of FIGS. 7B and 19) can be fabricated to form a generally planar multi-layer tissue. Human skin can be characterized as a multi-layer structure having three layers (Error! Reference source not found.5) [110] in which the middle layer is responsible for the mechanical properties of the skin [111]. The middle layer features interweaved collagen fibers embedded in elastin components. Inspired by the skin's properties, the sandwich scaffold may be designed with two or more layers in which (i) one or more textile fabric layer (e.g., middle layer) is designed to provide mechanical strength for the scaffolds and (ii) one or more outer layers (e.g., electrospun mats) are designed to protect the textile fabric layer and/or support the initial cell growth on the sandwich scaffold.

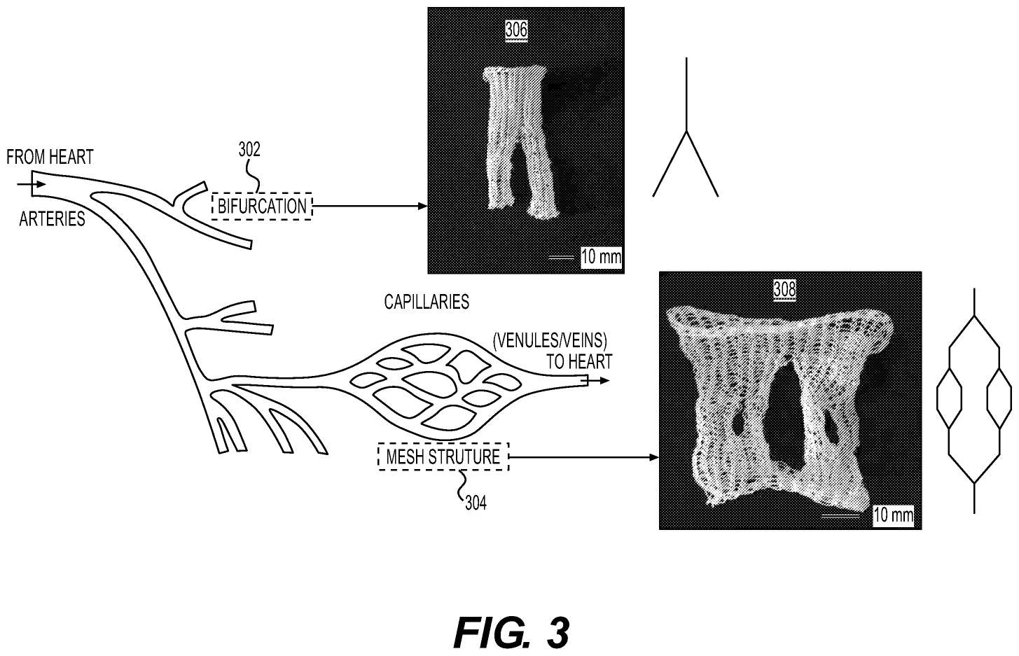

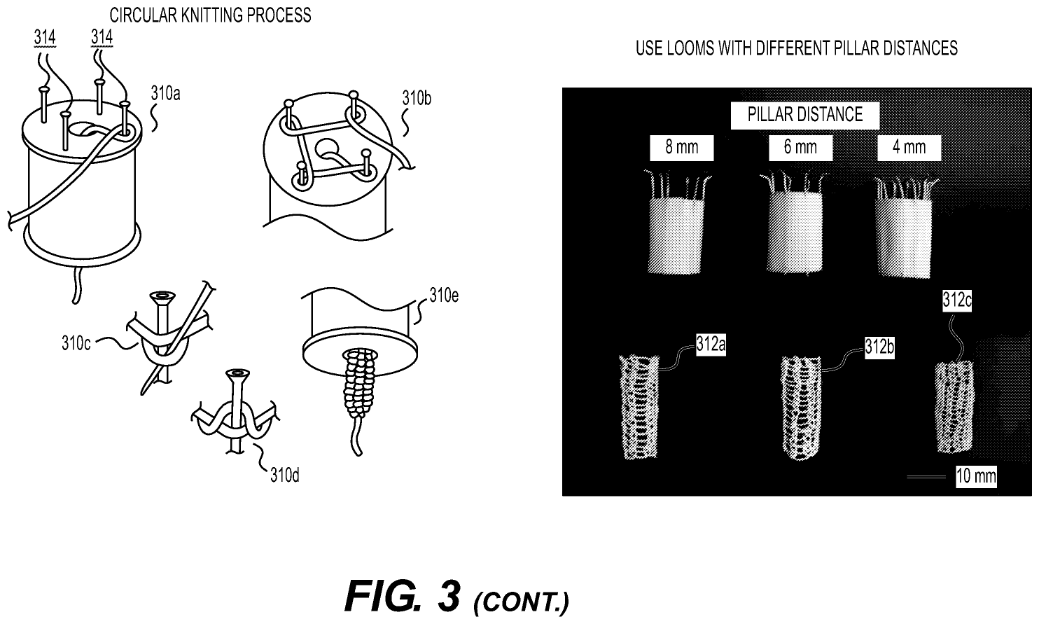

In additional embodiments, the textile patterns can be fabricated to form a pillar-shaped tissue, a mesh-shaped tissue, or a pillar mesh-shaped tissue (FIG. 3), e.g., for vascular tissues, among others. In the example shown in FIG. 3, vascular tissue may include bifurcations (302) or form mesh (304) structures. FIG. 3 shows example textile patterns formed as a pillar-shaped scaffold 306 and a pillar mesh-shaped scaffold 308.

To form the pillar-shaped structures (e.g., 306, 308), a circular knitting process (e.g., shown via diagrams 310a, 310b, 310c, 310d, and 310e) may be employed. FIG. 3 further shows different pillar structures having different pillar interstitial distances (312a, 312b, 312c) that can be fabricated by varying the pillar distance and number of anchors (314). In the example, the three pillars 312a, 312b, 312c, are fabricated via a circular knitting process using 6, 7, and 11 anchors, respectively.

Various textile patterns. The exemplary method can fabricate various textile patterns by using yarns developed in Examples 1, 2, 4, and 5.

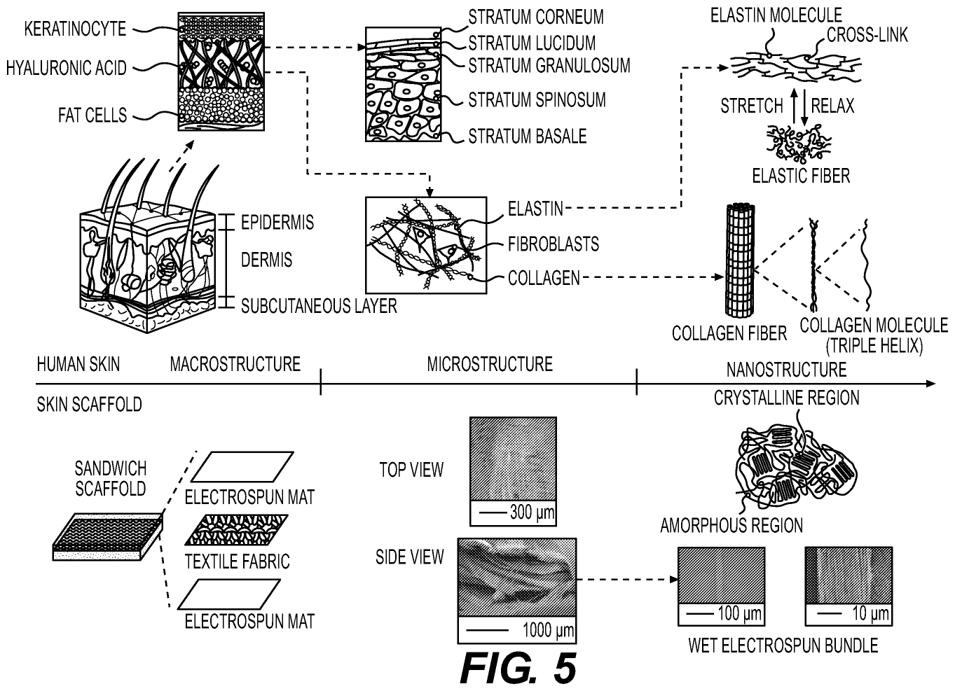

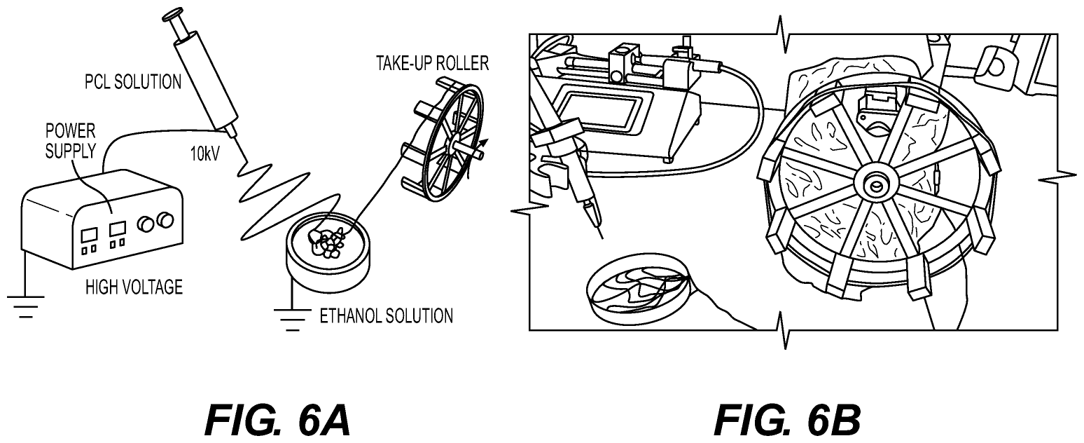

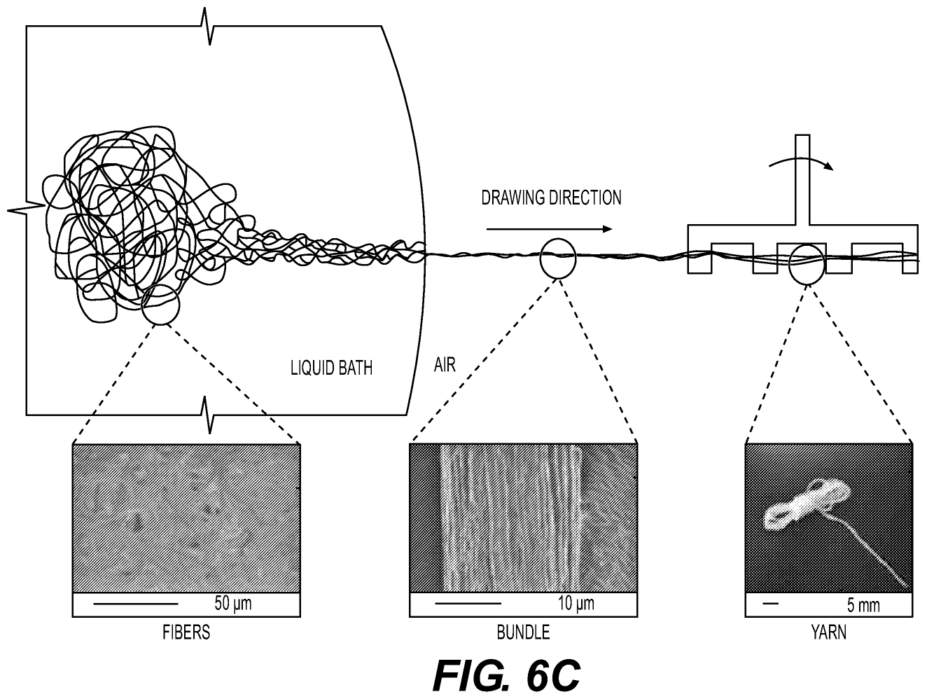

FIG. 5 shows example textile tools that can be employed in the method. Woven, knitted, and braided patterns may be fabricated (FIG. 6).

Preparation of sandwich scaffolds. The knitted fabrics may be used as the middle layer of the sandwich scaffolds in a set of experiments. The outer layers of the sandwich scaffold were fabricated by electrospinning on both sides of the textile fabric for the same time duration. In one example, the applied voltage may be set as 10 kV, the flow rate was set as 35 μl/min, and the distance between the nozzle tip to the collector's bottom was set as 150 mm. These parameters can be adjusted to achieve a desired or pre-fined fiber and yarn configuration.

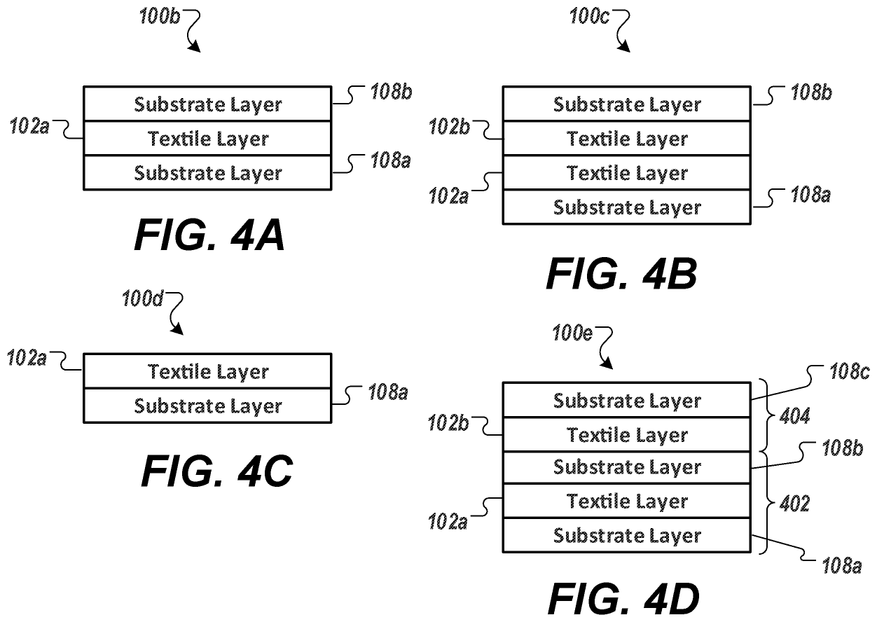

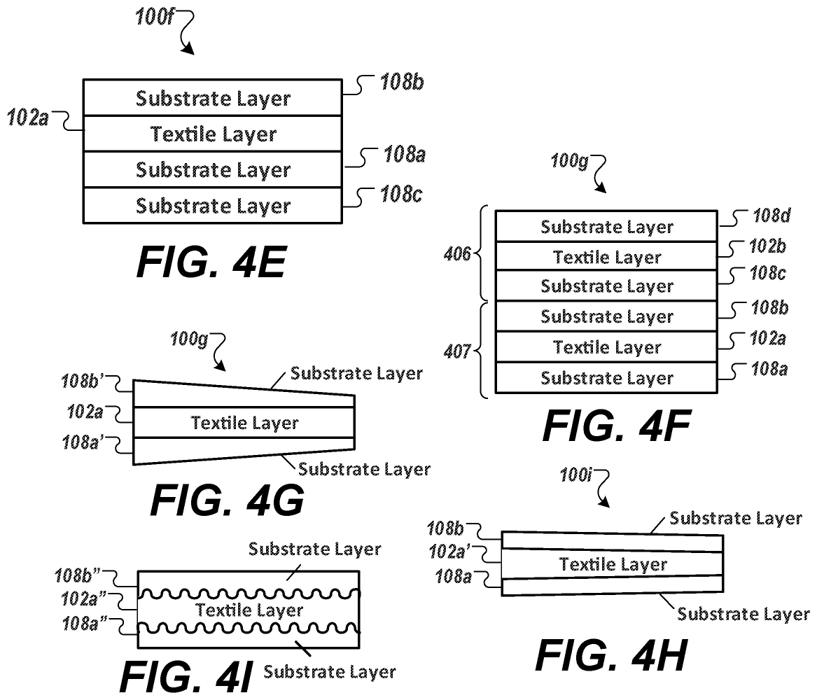

Various examples of the configurations of scaffold textile layer(s) 102 and the substrate layer(s) 108 of the tissue engineering system 100 are provided. FIGS. 4A-4I show exemplary scaffolded tissue engineering system 100 (shown as 100b, 100c, 100d, 100e, 100f, 100g, 100h, respectively) with one or more substrate layers (e.g., one or more outer layers) and one or more textile layers in different configurations.

In FIG. 4A, the textile layer 102 (shown as 102a) is formed between two substrate layers 108 (shown as 108a and 108b).

In FIG. 4B, two textile layers 102 (shown as 102a, 102b) are formed adjacent to one another. The two textile layers 102a, 102b may be fabricated separately and then attached together. The two textile layers 102a, 102b may be the form of the same type of polymer material or mixture or of different polymer material or mixture. The two textile layers 102a, 102b may have different fiber orientations, pore geometries, and/or surface topographies of the yarn and/or fibers.

In FIG. 4C, the textile layer 102 (shown as 102a) is formed with a substrate layer 108 (shown as 108a).

In FIG. 4D, two textile layers 102a, 102b are formed between three substrate layers 108a, 108b, and 108c. In some embodiments, a first set 402 of layers is fabricated comprising textile layer 102a and substrate layers 108a, 108b, and a second set 404 of layers is fabricated comprising textile layer 102b and substrate layers 108c. The first and second sets of layers 402, 404 may then be bonded or attached together.

In FIG. 4E, the textile layer 102 (shown as 102a) is formed between two substrate layers 108 (shown as 108a and 108b), and one of the substrate layers is bonded to a third substrate layer 108c.

In FIG. 4F, two sets (406, 408) of textile layer and substrate layers are formed and bonded or attached to one another in which each set includes a textile layer 102 (shown as 102a, 102b) and two substrate layers 108 (shown as 108a, 108b, 108c, and 108d).

In certain configurations, the thickness of the textile layer or the substrate layer may be varied (see FIGS. 4G, 4H, and 4I). In FIG. 4G, the textile layer 102 (shown as 102a) is formed between two substrate layers 108 (shown as 108a′ and 108b′), each having varying thicknesses. In FIG. 4H, the textile layer 102 (shown as 102a′) is formed, having a varying thickness and between two substrate layers 108 (shown as 108a and 108b). In FIG. 4I, the textile layer 102 (shown as 102a″) is formed, having varying thicknesses and between two substrate layers 108 (shown as 108a″ and 108b″).

The various configurations of FIGS. 4A-4I may be employed to provide specific mechanical properties for the scaffolded tissue engineering system 100.

Mechanical properties. The tensile testing results (Error! Reference source not found.12A-12E) showed that the strain-stiffening property of the textile fabric could be transmitted to the scaffold, which verifies that the middle layer can mimic the skin strain-stiffening property. The results also demonstrated that the wet electrospun yarns could be used in various textile processes, and the designed sandwich scaffolds can mimic the strain-stiffening property of skin tissues.

The following patents, applications, and publications as listed below are hereby incorporated by reference in their entirety herein.

### REFERENCES

[1] E. Schnell, K. Klinkhammer, S. Balzer, G. Brook, D. Klee, P. Dalton, J. Mey, Guidance of glial cell migration and axonal growth on electrospun nanofibers of poly-F-caprolactone and a collagen/poly-F-caprolactone blend, Biomaterials. 28 (2007) 3012-3025.
[2] Z. Yin, X. Chen, J. L. Chen, W. L. Shen, T. M. Hieu Nguyen, L. Gao, H. W. Ouyang, The regulation of tendon stem cell differentiation by the alignment of nanofibers, Biomaterials. 31 (2010) 2163-2175.
[3] H. Lu, Z. Feng, Z. Gu, C. Liu, Growth of outgrowth endothelial cells on aligned PLLA nanofibrous scaffolds, Journal of Materials Science: Materials in Medicine. 20 (2009) 1937-1944.
[4] X. Zong, H. Bien, C. Y. Chung, L. Yin, D. Fang, B. S. Hsiao, B. Chu, E. Entcheva, Electrospun fine-textured scaffolds for heart tissue constructs, Biomaterials. 26 (2005) 5330-5338.
[5] S. Sabetkish, A. M. Kajbafzadeh, N. Sabetkish, R. Khorramirouz, A. Akbarzadeh, S. L. Seyedian, P. Pasalar, S. Orangian, R. S. Hossein Beigi, Z. Aryan, H. Akbari, S. M. Tavangar, Whole-organ tissue engineering: Decellularization and recellularization of three-dimensional matrix liver scaffolds, Journal of Biomedical Materials Research—Part A. 103 (2015) 1498-1508.
[6] K. Wang, C. Wu, Z. Qian, C. Zhang, B. Wang, M. A. Vannan, Dual-material 3D printed metamaterials with tunable mechanical properties for patient-specific tissue-mimicking phantoms, Additive Manufacturing. 12 (2016) 31-37.
[7] D. Zhalmuratova, T. G. La, K. T. T. Yu, A. R. A. Szojka, S. H. J. Andrews, A. B. Adesida, C. Il Kim, D. S. Nobes, D. H. Freed, H. J. Chung, Mimicking “j-Shaped” and Anisotropic Stress-Strain Behavior of Human and Porcine Aorta by Fabric-Reinforced Elastomer Composites, ACS Applied Materials and Interfaces. 11 (2019) 33323-33335.
[8] Y. Ma, X. Feng, J. A. Rogers, Y. Huang, Y. Zhang, Design and application of “J-shaped” stress-strain behavior in stretchable electronics: A review, Lab on a Chip. 17 (2017) 1689-1704.
[9] A. Maziz, A. Concas, A. Khaldi, J. Stålhand, N. K. Persson, E. W. H. Jager, Knitting and weaving artificial muscles, Science Advances. 3 (2017) e1600327.
[10] M. Akbari, A. Tamayol, S. Bagherifard, L. Serex, P. Mostafalu, N. Faramarzi, M. H. Mohammadi, A. Khademhosseini, Textile Technologies and Tissue Engineering: A Path Toward Organ Weaving, Advanced Healthcare Materials. 5 (2016) 751-766.
[11] M. Younesi, A. Islam, V. Kishore, J. M. Anderson, O. Akkus, Tenogenic Induction of Human MSCs by Anisotropically Aligned Collagen Biotextiles, Advanced Functional Materials. 24 (2014) 5762-5770.
[12] M. Cěpič, Knitted patterns as a model for anisotropy, Physics Education. 47 (2012) 456-461.
[13] F. T. Moutos, B. T. Estes, F. Guilak, Multifunctional Hybrid Three-dimensionally Woven Scaffolds for Cartilage Tissue Engineering, Macromolecular Bioscience. 10 (2010) 1355-1364.
[14] I. C. Liao, F. T. Moutos, B. T. Estes, X. Zhao, F. Guilak, Composite three-dimensional woven scaffolds with interpenetrating network hydrogels to create functional synthetic articular cartilage, Advanced Functional Materials. 23 (2013) 5833-5839.
[15] F. T. Moutos, L. E. Freed, F. Guilak, A biomimetic three-dimensional woven composite scaffold for functional tissue engineering of cartilage, Nature Materials. 6 (2007) 162-167.
[16] F. T. Moutos, F. Guilak, Functional properties of cell-seeded three-dimensionally woven poly(ε-Caprolactone) scaffolds for cartilage tissue engineering, Tissue Engineering—Part A. 16 (2010) 1291-1301.
[17] H. H. Lu, J. A. Cooper, S. Manuel, J. W. Freeman, M. A. Attawia, F. K. Ko, C. T. Laurencin, Anterior cruciate ligament regeneration using braided biodegradable scaffolds: In vitro optimization studies, Biomaterials. 26 (2005) 4805-4816.
[18] H. Onoe, T. Okitsu, A. Itou, M. Kato-Negishi, R. Gojo, D. Kiriya, K. Sato, S. Miura, S. Iwanaga, K. Kuribayashi-Shigetomi, Y. T. Matsunaga, Y. Shimoyama, S. Takeuchi, Metre-long cell-laden microfibres exhibit tissue morphologies and functions, Nature Materials. 12 (2013) 584-590.
[19] J. Boublik, H. Park, M. Radisic, E. Tognana, F. Chen, M. Pei, G. Vunjak-Novakovic, L. E. Freed, Mechanical properties and remodeling of hybrid cardiac constructs made from heart cells, fibrin, and biodegradable, elastomeric knitted fabric, Tissue Engineering. 11 (2005) 1122-1132.
[20] G. Chen, T. Sato, T. Ushida, R. Hirochika, Y. Shirasaki, N. Ochiai, T. Tateishi, The use of a novel PLGA fiber/collagen composite web as a scaffold for engineering of articular cartilage tissue with adjustable thickness, Journal of Biomedical Materials Research—Part A. 67 (2003) 1170-1180.
[21] C. Vaquette, C. Kahn, C. Frochot, C. Nouvel, J. L. Six, N. De Isla, L. H. Luo, J. Cooper-White, R. Rahouadj, X. Wang, Aligned poly(L-lactic-co-e-caprolactone) electrospun microfibers and knitted structure: A novel composite scaffold for ligament tissue engineering, Journal of Biomedical Materials Research—Part A. 94 (2010) 1270-1282.
[22] S. Sahoo, S. L. Toh, J. C. H. Goh, A bFGF-releasing silk/PLGA-based biohybrid scaffold for ligament/tendon tissue engineering using mesenchymal progenitor cells, Biomaterials. 31 (2010) 2990-2998.
[23] A. Wang, Q. Ao, W. Cao, M. Yu, Q. He, L. Kong, L. Zhang, Y. Gong, X. Zhang, Porous chitosan tubular scaffolds with knitted outer wall and controllable inner structure for nerve tissue engineering, Journal of Biomedical Materials Research—Part A. 79 (2006) 36-46.
[24] G. Chen, T. Sato, H. Ohgushi, T. Ushida, T. Tateishi, J. Tanaka, Culturing of skin fibroblasts in a thin PLGA-collagen hybrid mesh, Biomaterials. 26 (2005) 2559-2566.
[25] K. W. Ng, D. W. Hutmacher, Reduced contraction of skin equivalent engineered using cell sheets cultured in 3D matrices, Biomaterials. 27 (2006) 4591-4598.
[26] H. Ahn, K. J. Kim, S. Y. Park, J. E. Huh, H. J. Kim, W. R. Yu, 3D braid scaffolds for regeneration of articular cartilage, Journal of the Mechanical Behavior of Biomedical Materials. 34 (2014) 37-46.
[27] J. A. Cooper, H. H. Lu, F. K. Ko, J. W. Freeman, C. T. Laurencin, Fiber-based tissue-engineered scaffold for ligament replacement: Design considerations and in vitro evaluation, Biomaterials. 26 (2005) 1523-1532.
[28] J. G. Barber, A. M. Handorf, T. J. Allee, W. J. Li, Braided nanofibrous scaffold for tendon and ligament tissue engineering, Tissue Engineering—Part A. 19 (2013) 1265-1274.
[29] G. H. Altman, R. L. Horan, H. H. Lu, J. Moreau, I. Martin, J. C. Richmond, D. L. Kaplan, Silk matrix for tissue engineered anterior cruciate ligaments, Biomaterials. 23 (2002) 4131-4141.
[30] A. Ide, M. Sakane, G. Chen, H. Shimojo, T. Ushida, T. Tateishi, Y. Wadano, Y. Miyanaga, Collagen hybridization with poly(L-lactic acid) braid promotes ligament cell migration, Materials Science and Engineering C. 17 (2001) 95-99.
[31] J. W. Freeman, M. D. Woods, C. T. Laurencin, Tissue engineering of the anterior cruciate ligament using a braid-twist scaffold design, Journal of Biomechanics. 40 (2007) 2029-2036.
[32] C. T. Laurencin, J. W. Freeman, Ligament tissue engineering: An evolutionary materials science approach, Biomaterials. 26 (2005) 7530-7536.
[33] C. P. Laurent, D. Durville, D. Mainard, J. F. Ganghoffer, R. Rahouadj, A multilayer braided scaffold for Anterior Cruciate Ligament: Mechanical modeling at the fiber scale, Journal of the Mechanical Behavior of Biomedical Materials. 12 (2012) 184-196.
[34] M. Sato, M. Maeda, H. Kurosawa, Y. Inoue, Y. Yamauchi, H. Iwase, Reconstruction of rabbit Achilles tendon with three bioabsorbable materials: Histological and biomechanical studies, Journal of Orthopaedic Science. 5 (2000) 256-267.
[35] M. C. Lu, Y. T. Huang, J. H. Lin, C. H. Yao, C. W. Lou, C. C. Tsai, Y. S. Chen, Evaluation of a multi-layer microbraided polylactic acid fiber-reinforced conduit for peripheral nerve regeneration, Journal of Materials Science: Materials in Medicine. 20 (2009) 1175-1180.
[36] B. Wang, P. Zhang, W. Song, L. Zhao, C. he, Design and properties of a new braided poly lactic-co-glycolic acid catheter for peripheral nerve regeneration, Textile Research Journal. 85 (2015) 51-61.
[37] Y. Wu, L. Wang, B. Guo, P. X. Ma, Interwoven Aligned Conductive Nanofiber Yarn/Hydrogel Composite Scaffolds for Engineered 3D Cardiac Anisotropy, ACS Nano. 11 (2017) 5646-5659.
[38] S. Wu, B. Duan, X. Qin, J. T. Butcher, Living nano-micro fibrous woven fabric/hydrogel composite scaffolds for heart valve engineering, Acta Biomaterialia. 51 (2017) 89-100.
[39] F. Han, S. Liu, X. Liu, Y. Pei, S. Bai, H. Zhao, Q. Lu, F. Ma, D. L. Kaplan, H. Zhu, Woven silk fabric-reinforced silk nanofibrous scaffolds for regenerating load-bearing soft tissues, Acta Biomaterialia. 10 (2014) 921-930.
[40] X. Wang, Q. Li, X. Hu, L. Ma, C. You, Y. Zheng, H. Sun, C. Han, C. Gao, Fabrication and characterization of poly(1-lactide-co-glycolide) knitted mesh-reinforced collagen-chitosan hybrid scaffolds for dermal tissue engineering, Journal of the Mechanical Behavior of Biomedical Materials. 8 (2012) 204-215.
[41] G. Chen, T. Ushida, T. Tateishi, A hybrid network of synthetic polymer mesh and collagen sponge, Chemical Communications. (2000) 1505-1506.
[42] M. Ananta, C. E. Aulin, M. Sc, D. Ph, D. Aibibu, A Poly (Lactic Acid-Co-Caprolactone)-Collagen Hybrid, Tissue Engineering Part A. 15 (2009) 1667-1675.
[43] W. Shao, J. He, Q. Han, F. Sang, Q. Wang, L. Chen, S. Cui, B. Ding, A biomimetic multilayer nanofiber fabric fabricated by electrospinning and textile technology from polylactic acid and Tussah silk fibroin as a scaffold for bone tissue engineering, Materials Science and Engineering C. 67 (2016) 599-610.
[44] Y. S. Gloy, M. Loehrer, B. Lang, L. Rongen, T. Gries, S. Jockenhoevel, Tubular woven narrow fabrics for replacement of cruciate ligaments, Annals of Biomedical Engineering. 41 (2013) 1950-1956.
[45] X. Chen, I. Zanini, An experimental investigation into the structure and mechanical properties of 3D woven orthogonal structures, Journal of the Textile Institute. 88 (1997) 449-464.
[46] O. Kononova, A. Krasnikovs, K. Dzelzitis, G. Kharkova, A. Vagel, M. Eiduks, Silmuskootud puuvillakangast komposiitide mehaaniliste omaduste modelleerimine ja võrdlemine katsetulemustega, Estonian Journal of Engineering. 17 (2011) 39-50.
[47] S. Wu, B. Duan, P. Liu, C. Zhang, X. Qin, J. T. Butcher, Fabrication of Aligned Nanofiber Polymer Yarn Networks for Anisotropic Soft Tissue Scaffolds, ACS Applied Materials and Interfaces. 8 (2016) 16950-16960.
[48] K. A. Jones, A. Feola, L. Meyn, S. D. Abramowitch, P. A. Moalli, Tensile properties of commonly used prolapse meshes, International Urogynecology Journal. 20 (2009) 847-853.
[49] T. Yagi, M. Sato, Y. Nakazawa, K. Tanaka, M. Sata, K. Itoh, Y. Takagi, T. Asakura, Preparation of double-raschel knitted silk vascular grafts and evaluation of short-term function in a rat abdominal aorta, Journal of Artificial Organs. 14 (2011) 89-99.
[50] H. W. Ouyang, S. L. Toh, J. Goh, T. E. Tay, K. Moe, Assembly of bone marrow stromal cell sheets with knitted poly (L-lactide) scaffold for engineering ligament analogs, Journal of Biomedical Materials Research—Part B Applied Biomaterials. 75 (2005) 264-271.
[51] P. A. Madurantakam, C. P. Cost, D. G. Simpson, G. L. Bowlin, Science of nanofibrous scaffold fabrication: Strategies for next generation tissue-engineering scaffolds, Nanomedicine. 4 (2009) 193-206.
[52] J. Xue, T. Wu, Y. Dai, Y. Xia, Electrospinning and electrospun nanofibers: Methods, materials, and applications, Chemical Reviews. 119 (2019) 5298-5415.
[53] H. G. Sundararaghavan, J. A. Burdick, Gradients with depth in electrospun fibrous scaffolds for directed cell behavior, Biomacromolecules. 12 (2011) 2344-2350.
[54] S. Wu, Y. Wang, P. N. Streubel, B. Duan, Living nanofiber yarn-based woven biotextiles for tendon tissue engineering using cell tri-culture and mechanical stimulation, Acta Biomaterialia. 62 (2017) 102-115.
[55] J. Xie, M. R. MacEwan, W. Z. Ray, W. Liu, D. Y. Siewe, Y. Xia, Radially aligned, electrospun nanofibers as dural substitutes for wound closure and tissue regeneration applications, ACS Nano. 4 (2010) 5027-5036.
[56] B. Zhang, B. Seong, J. Lee, V. Nguyen, D. Cho, D. Byun, One-Step Sub-micrometer-Scale Electrohydrodynamic Inkjet Three-Dimensional Printing Technique with Spontaneous Nanoscale Joule Heating, ACS Applied Materials and Interfaces. 9 (2017) 29965-29972.
[57] B. Seong, H. Yoo, V. D. Nguyen, Y. Jang, C. Ryu, D. Byun, Metal-mesh based transparent electrode on a 3-D curved surface by electrohydrodynamic jet printing, Journal of Micromechanics and Microengineering. 24 (2014).
[58] A. P. Tenggara, S. J. Park, H. T. Yudistira, Y. H. Ahn, D. Byun, Fabrication of terahertz metamaterials using electrohydrodynamic jet printing for sensitive detection of yeast, Journal of Micromechanics and Microengineering. 27 (2017).
[59] H. Liang, J. He, J. Chang, B. Zhang, D. Li, Coaxial nozzle-assisted electrohydrodynamic printing for microscale 3D cell-laden constructs, International Journal of Bioprinting. 4 (2018).
[60] M. Kim, H. S. Yun, G. H. Kim, Electric-field assisted 3D-fibrous bioceramic-based scaffolds for bone tissue regeneration: Fabrication, characterization, and in vitro cellular activities, Scientific Reports. 7 (2017) 3166.
[61] Q. Lei, J. He, D. Li, Electrohydrodynamic 3D printing of layer-specifically oriented, multiscale conductive scaffolds for cardiac tissue engineering, Nanoscale. 11 (2019) 15195-15205.
[62] Y. L. and D. L. Xiang Zhao, Jiankang He, Fangyuan Xu, Electrohydrodynamic printing: a potential tool for high-resolution hydrogel/cell patterning, Virtual and Physical Prototyping. 11 (2016) 57-63.
[63] Z. Tan, T. Liu, J. Zhong, Y. Yang, W. Tan, Control of cell growth on 3D-printed cell culture platforms for tissue engineering, Journal of Biomedical Materials Research—Part A. 105 (2017) 3281-3292.
[64] M. S. Kim, G. Kim, Electrohydrodynamic jet process for pore-structure-controlled 3D fibrous architecture as a tissue regenerative material: Fabrication and cellular activities, Langmuir. 30 (2014) 8551-8557.
[65] V. D. Nguyen, D. Byun, Mechanism of electrohydrodynamic printing based on ac voltage without a nozzle electrode, Applied Physics Letters. 94 (2009) 173509.
[66] G. S. Bisht, G. Canton, A. Mirsepassi, L. Kulinsky, S. Oh, D. Dunn-Rankin, M. J. Madou, Controlled continuous patterning of polymeric nanofibers on three-dimensional substrates using low-voltage near-field electrospinning, Nano Letters. 11 (2011) 1831-1837.
[67] K. H. Lee, H. Y. Kim, H. J. Bang, Y. H. Jung, S. G. Lee, The change of bead morphology formed on electrospun polystyrene fibers, Polymer. 44 (2003) 4029-4034.
[68] E. T. Den Braber, J. E. De Ruijter, L. A. Ginsel, A. F. Von Recum, J. A. Jansen, Quantitative analysis of fibroblast morphology on microgrooved surfaces with various groove and ridge dimensions, Biomaterials. 17 (1996) 2037-2044.
[69] G. H. Kim, S. H. Ahn, H. J. Lee, S. Lee, Y. Cho, W. Chun, A new hybrid scaffold using rapid prototyping and electrohydrodynamic direct writing for bone tissue regeneration, Journal of Materials Chemistry. 21 (2011) 19138-19143.
[70] M. Rasekh, Z. Ahmad, C. C. Frangos, L. Bozec, M. Edirisinghe, R. M. Day, Spatial and temporal evaluation of cell attachment to printed polycaprolactone microfibres, Acta Biomaterialia. 9 (2013) 5052-5062.
[71] X. Wang, M. Min, Z. Liu, Y. Yang, Z. Zhou, M. Zhu, Y. Chen, B. S. Hsiao, Poly(ethyleneimine) nanofibrous affinity membrane fabricated via one step wet-electrospinning from poly(vinyl alcohol)-doped poly(ethyleneimine) solution system and its application, Journal of Membrane Science. 379 (2011) 191-199.
[72] S. Farzamfar, M. Naseri-Nosar, A. Vaez, F. Esmaeilpour, A. Ehterami, H. Sahrapeyma, H. Samadian, A. A. Hamidieh, S. Ghorbani, A. Goodarzi, A. Azimi, M. Salehi, Neural tissue regeneration by a gabapentin-loaded cellulose acetate/gelatin wet-electrospun scaffold, Cellulose. 25 (2018) 1229-1238.
[73] Y. Zheng, J. Miao, N. Maeda, D. Frey, R. J. Linhardt, T. J. Simmons, Uniform nanoparticle coating of cellulose fibers during wet electrospinning, Journal of Materials Chemistry A. 2 (2014) 15029-15034.
[74] J. Rnjak-Kovacina, A. S. Weiss, Increasing the pore size of electrospun scaffolds, Tissue Engineering—Part B: Reviews. 17 (2011) 365-372.
[75] C. S. Ki, S. Y. Park, H. J. Kim, H. M. Jung, K. M. Woo, J. W. Lee, Y. H. Park, Development of 3-D nanofibrous fibroin scaffold with high porosity by electrospinning: Implications for bone regeneration, Biotechnology Letters. 30 (2008) 405-410.
[76] M. Zhang, H. Lin, Y. Wang, G. Yang, H. Zhao, D. Sun, Fabrication and durable antibacterial properties of 3D porous wet electrospun RCSC/PCL nanofibrous scaffold with silver nanoparticles, Applied Surface Science. 414 (2017) 52-62.
[77] E. Smit, U. Buttner, R. D. Sanderson, Continuous yarns from electrospun fibers, Polymer. 46 (2005) 2419-2423.
[78] H. Wang, L. Kong, G. R. Ziegler, Aligned wet-electrospun starch fiber mats, Food Hydrocolloids. 90 (2019) 113-117.
[79] M. S. Khil, S. R. Bhattarai, H. Y. Kim, S. Z. Kim, K. H. Lee, Novel fabricated matrix via electrospinning for tissue engineering, Journal of Biomedical Materials Research—Part B Applied Biomaterials. 72 (2005) 117-124.
[80] L. Wang, Y. Wu, B. Guo, P. X. Ma, Nanofiber Yarn/Hydrogel Core-Shell Scaffolds Mimicking Native Skeletal Muscle Tissue for Guiding 3D Myoblast Alignment, Elongation, and Differentiation, ACS Nano. 9 (2015) 9167-9179.
[81] J. P. Spatz, B. Geiger, Molecular Engineering of Cellular Environments: Cell Adhesion to Nano-Digital Surfaces, Methods in Cell Biology. 83 (2007) 89-111.
[82] D. E. Discher, P. Janmey, Y. L. Wang, Tissue cells feel and respond to the stiffness of their substrate, Science. 310 (2005) 1139-1143.
[83] J. Zeltinger, J. K. Sherwood, D. A. Graham, R. Müeller, L. G. Griffith, Effect of pore size and void fraction on cellular adhesion, proliferation, and matrix deposition, Tissue Engineering. 7 (2001) 557-572.
[84] S. H. Bang, T. H. Kim, H. Y. Lee, U. S. Shin, H. W. Kim, Nanofibrous-structured biopolymer scaffolds obtained by a phase separation with camphene and initial cellular events, Journal of Materials Chemistry. 21 (2011) 4523-4530.
[85] A. Barbetta, A. Carrino, M. Costantini, M. Dentini, Polysaccharide based scaffolds obtained by freezing the external phase of gas-in-liquid foams, Soft Matter. 6 (2010) 5213-5224.
[86] S. H. Ahn, H. J. Lee, G. H. Kim, Polycaprolactone scaffolds fabricated with an advanced electrohydrodynamic direct-printing method for bone tissue regeneration, Biomacromolecules. 12 (2011) 4256-4263.
[87] D. Yao, S. Dong, Q. Lu, X. Hu, D. L. Kaplan, B. Zhang, H. Zhu, Salt-leached silk scaffolds with tunable mechanical properties, Biomacromolecules. 13 (2012) 3723-3729.
[88] R. Gauvin, Y. C. Chen, J. W. Lee, P. Soman, P. Zorlutuna, J. W. Nichol, H. Bae, S. Chen, A. Khademhosseini, Microfabrication of complex porous tissue engineering scaffolds using 3D projection stereolithography, Biomaterials. 33 (2012) 3824-3834.
[89] J. Y. Kim, C. Lee, K. Park, G. Lim, C. Kim, Fast optical-resolution photoacoustic microscopy using a 2-axis water-proofing MEMS scanner, Scientific Reports. 5 (2015) 7932.
[90] L. E. Bertassoni, M. Cecconi, V. Manoharan, M. Nikkhah, J. Hjortnaes, A. L. Cristino, G. Barabaschi, D. Demarchi, M. R. Dokmeci, Y. Yang, A. Khademhosseini, Hydrogel bioprinted microchannel networks for vascularization of tissue engineering constructs, Lab on a Chip. 14 (2014) 2202-2211.
[91] D. B. Kolesky, R. L. Truby, A. S. Gladman, T. A. Busbee, K. A. Homan, J. A. Lewis, 3D bioprinting of vascularized, heterogeneous cell-laden tissue constructs, Advanced Materials. 26 (2014) 3124-3130.
[92] H. Qi, M. Ghodousi, Y. Du, C. Grun, H. Bae, P. Yin, A. Khademhosseini, DNA-directed self-assembly of shape-controlled hydrogels, Nature Communications. 4 (2013) 2275.
[93] Y. Du, E. Lo, S. Ali, A. Khademhosseini, Directed assembly of cell-laden microgels for fabrication of 3D tissue constructs, Proceedings of the National Academy of Sciences of the United States of America. 105 (2008) 9522-9527.
[94] J. J. Norman, T. A. Desai, Methods for fabrication of nanoscale topography for tissue engineering scaffolds, Annals of Biomedical Engineering. 34 (2006) 89-101.
[95] Y. Duan, Y. Huang, Z. Yin, N. Bu, W. Dong, Non-wrinkled, highly stretchable piezoelectric devices by electrohydrodynamic direct-writing, Nanoscale. 6 (2014) 3289-3295.
[96] Y. Huang, N. Bu, Y. Duan, Y. Pan, H. Liu, Z. Yin, Y. Xiong, Electrohydrodynamic direct-writing, Nanoscale. 5 (2013) 12007-12017.
[97] M. Lee, H. Y. Kim, Toward nanoscale three-dimensional printing: Nanowalls built of electrospun nanofibers, Langmuir. 30 (2014) 1210-1214.
[98] M. S. Onses, E. Sutanto, P. M. Ferreira, A. G. Alleyne, J. A. Rogers, Mechanisms, Capabilities, and Applications of High-Resolution Electrohydrodynamic Jet Printing, Small. 11 (2015) 4237-4266.
[99] M. Cloupeau, B. Prunet-Foch, Electrohydrodynamic spraying functioning modes: a critical review, Journal of Aerosol Science. 25 (1994) 1021-1036.
[100] J. H. He, Y. Wu, W. W. Zuo, Critical length of straight jet in electrospinning, Polymer. 46 (2005) 12637-12640.
[101] M. Yousefzadeh, Modeling and simulation of the electrospinning process, in: Electrospun Nanofibers, 2017: pp. 277-301.
[102] Y. M. Shin, M. M. Hohman, M. P. Brenner, G. C. Rutledge, Experimental characterization of electrospinning: The electrically forced jet and instabilities, Polymer. 42 (2001) 09955-09967.
[103] J. H. He, Y. Q. Wan, Allometric scaling for voltage and current in electrospinning, Polymer. 45 (2004) 6731-6734.
[104] H. H. Huang, C. T. Ho, T. H. Lee, T. L. Lee, K. K. Liao, F. L. Chen, Effect of surface roughness of ground titanium on initial cell adhesion, Biomolecular Engineering. 21 (2004) 93-97.
[105] B. D. Boyan, R. Batzer, K. Kieswetter, Y. Liu, D. L. Cochran, S. Szmuckler-Moncler, D. D. Dean, Z. Schwartz, Titanium surface roughness alters responsiveness of MG63 osteoblast-like cells to 1α,25-(OH)2D3, Journal of Biomedical Materials Research. 39 (1997) 77-85.
[106] Y. Yokoyama, S. Hattori, C. Yoshikawa, Y. Yasuda, H. Koyama, T. Takato, H. Kobayashi, Novel wet electrospinning system for fabrication of spongiform nanofiber 3-dimensional fabric, Materials Letters. 63 (2009) 754-756.
[107] J. Heo, H. Nam, D. Hwang, S. J. Cho, S. Y. Jung, D. W. Cho, J. H. Shim, G. Lim, Enhanced cellular distribution and infiltration in a wet electrospun three-dimensional fibrous scaffold using eccentric rotation-based hydrodynamic conditions, Sensors and Actuators, B: Chemical. 226 (2016) 357-363.
[108] D 3039/D3039M ASTM, Standard Test Method for Tensile Properties of Polymer Matrix Composite Materials, Annual Book of ASTM Standards. 15.03 (2000) 105-116.
[109] G. Vazquez, E. Alvarez, J. M. Navaza, Surface Tension of Alcohol+Water from 20 to 50° C., Journal of Chemical and Engineering Data. 40 (1995) 611-614.
[110] S. MacNeil, Progress and opportunities for tissue-engineered skin, Nature. 445 (2007) 874-880.
[111] M. L. Crichton, B. C. Donose, X. Chen, A. P. Raphael, H. Huang, M. A. F. Kendall, The viscoelastic, hyperelastic and scale dependent behaviour of freshly excised individual skin layers, Biomaterials. 32 (2011) 4670-4681.

### Example 4: Textile-Based Sandwich Scaffold Using Wet Electrospun Yarns for Skin Tissue Engineering

The demand for organ repair and transplantation, and a shortage of available donors, necessitates the clinical need to develop innovative strategies for repair and regeneration of injured or diseased tissues and organs. Tissue engineering, which aims to create living biological substitutes, offers promising avenues to address the organ shortage. One of the key elements in tissue engineering is to design and fabricate scaffolds with tissue-like properties. Failure to mimic properties of human tissues can have negative results in tissue integration and regeneration. A challenge in scaffold fabrication is to mimic the mechanical properties of human tissues because human tissues have strain-stiffening property (Jaspers et al., 2014; Motte and Kaufman, 2013; Wang et al., 2012a), while most synthetic biopolymers have strain-softening property (Jaspers et al., 2017; Wang et al., 2016).

Studies have shown that the strain-stiffening property of human tissues is associated with the helical structure of collagen and other fibrillar proteins (Piechocka et al., 2010; Wu et al., 2019). Inspired by the structure-property relationship of human tissues, researchers have used corrugations such as wrinkled sheets and wavy fibers in the scaffold structure to obtain the strain-stiffening property (Zhalmuratova et al., 2019). Z Wang et al. applied a cyclic tensile force to induce wavy nanofibers in bulk material and produced a strain-stiffening material (Wang et al., 2015b). K Wang et al. used multi-material 3-dimensional (3D) printing to embed helical microfibers into a soft polymeric matrix to generate tissue-mimicking phantoms (Wu et al., 2016).

However, the current research focuses mainly on the corrugation structure at a specific scale, either the nano- or the micro-scale, and neglects the hierarchical organization of human tissues. Human tissues have a distinct hierarchical organization at different structure levels from the nano- to macro-scale. For example, in human skin (FIG. 5), collagen molecules wind into the triple helix and then pack into collagen fibers. Collagen fibers interweave with each other and embed in the elastin matrix to form the middle layer of the skin tissue. This hierarchical organization of skin results in high sensitivity and high responsiveness towards stress.

Inspired by the corrugation structure and hierarchical organization in human tissues, some researchers have applied textile technology in tissue engineering. Using textile techniques allows easy control over yarn corrugation, and the resulting textile fabrics will have the hierarchical organization of a fiber-bundle yarn. Freeman et al. twisted fibers into yarns, then braided the yarns into a scaffold that exhibited similar strain-stiffening behavior to the anterior cruciate ligament (ACL) (Freeman et al., 2007). Maziz et al. used weaving and knitting methods to fabricate artificial muscles that had strain-stiffening properties and tunable mechanical strength (Maziz et al., 2017). Despite the progress in applying textile technology in tissue engineering (Akbari et al., 2016; Mironov et al., 2009; Pedde et al., 2017), most researchers used commercial yarns with a limited range of material properties that are hard to customize. Fabrication of yarns with sufficient mechanical strength and high biocompatibility has been very challenging due to limitations of the current widely-used yarn fabrication methods such as melt spinning, microfluidic spinning, wet spinning, electrospinning, etc. (Tamayol et al., 2013). To address this challenge, some researchers replaced the solid collector in the electrospinning device with a liquid collector, then drew the electrospun fibers from the liquid to get the wet electrospun yarn with the desired properties for tissue engineering applications (Khil et al., 2005; Wang et al., 2015a; Wu et al., 2017). However, to date, related research has been limited. The relationship between process parameters and the resulting wet electrospun yarn properties has not been fully understood, and the properties of wet electrospun yarns with other textile patterns beyond the woven pattern have yet to be investigated. Thus, to fulfill the potential of textile technology in mimicking the strain-stiffening property of human tissues, there is a great need to thoroughly analyze the wet electrospinning process and explore other textile patterns for tissue engineering.

In a study, a method fabricated a textile-based sandwich scaffold for skin tissue engineering. The study first fabricated polycaprolactone (PCL) yarns by wet electrospinning. Endothelial cells seeded on the wet electrospun yarns were able to align and elongate along the yarn direction. Then, the study crocheted the yarns into a textile fabric. The study designed a sandwich scaffold composed of the textile fabric in the middle layer and two electrospun mats in the outer layers. The thickness of the outer layers of the sandwich scaffold was also optimized by controlling the duration of the electrospinning process to facilitate the initial cell attachment and long-term cell growth. The textile-based sandwich scaffold had similar hierarchical organizations and interconnected structures to human skin. The scaffold exhibited strain-stiffening behavior, and its maximum stress, maximum strain, and elastic modulus can be adjusted to fall within the ranges of those of human skin. Cells seeded on the surface of the scaffold were able to proliferate on and penetrate the scaffold after culturing for 7 days. The proposed textile-based sandwich scaffold provides a powerful tool to mimic the physical, mechanical, and biological properties of human skin and other tissues.

### Materials and Methods

Materials and experimental setup: Polycaprolactone (PCL, Mn=80, 000, product number: 440744), Dichloromethane (DCM, product number: 270997), N, N-dimethylformamide (DMF, product number: 227056), and Glutaraldehyde solution (GA, product number: G6257) were purchased from Sigma-Aldrich®. The electrospun solution was prepared by dissolving PCL in the DCM: DMF (V/V ratio: 2:1) cosolvent at a weight concentration of 10%. The prepared electrospun solution was loaded into a syringe that was connected to a syringe pump (LEGATO 100, KD Scientific Co., USA). High voltage (PS/FJ30R04.0, Glassman High Voltage Co., USA) was applied at the nozzle (21G).

Yarn fabrication: To fabricate PCL yarns, PCL fibers were electrospun in a liquid bath, fibers were extracted out of the liquid bath via a tweezer and then collected on a take-up roller (FIG. 6A). The fibers formed into a bundle as they were drawn out of the liquid bath. The method fabricated a yarn from the bundle after collecting the bundle on the take-up roller for 20 min (FIG. 6B). The electrospinning process was carried out in the ambient environment. The electrospinning voltage was set at 10 kV, the flow rate at 40 l/min, and the distance between the nozzle tip to the container bottom at 40 mm. The method attached a piece of aluminous foil to the container bottom, then grounded the aluminous foil. Ethanol/water mixtures with different ratios were used as liquid baths. The height of the liquid in the container was set at 5 mm. The process of the fiber deposition in the liquid bath was monitored and recorded by a super-speed camera (SMM-C012-U, Mightex Co., USA). The take-up roller was controlled by a stepping motor (Silverpak 17C, LIN ENGINEERING, USA).

Characterization of wet electrospun yarns: The morphologies of the wet electrospun yarns were analyzed by a scanning electron microscope (SEM, SU8010, Hitachi, Japan) at an accelerating voltage of 3 kV after vacuum drying (Isotemp® 282A, Fisher Scientific, USA) and gold sputtering (Quorum Q-150T ES, USA). Quantitative data on fiber alignment was measured and analyzed using Image J software (NIH, USA). The thermal properties of the wet electrospun yarns were analyzed under a differential scanning calorimetry (DSC, Chip DSC 10, Linseis, Germany) by heating the samples to 100° C. at a heating rate of 10° C./min under the nitrogen flow. The degree of crystallinity was estimated by assuming that the melting enthalpy per unit mass of a pure crystalline PCL sample was identical to that of a 100% crystalline PCL sample (139.5 J/g) (Gupta et al., 2012).

Cell culture: Human Umbilical Vein Endothelial Cells (HUVECs, PromoCell, category number: C-12200) were used for experiments. HUVECs were cultured in Endothelial Cell Growth Medium (PromoCell, category number: C-22010). Cells were expanded under standard culture conditions in a humidified atmosphere with 5% CO2 at 37° C.

Cell cultivation on wet electrospun yarns: To investigate the biological properties of wet electrospun yarns, the method seeded cells on yarns and cultured cell-laden yarns. Before seeding cells, the method cut yarns into 10 mm and placed them in a 24-well plate. Then, the method sterilized the yarns by exposing them to the ultraviolet light for 30 min. Passage 15 HUVECs were harvested and suspended in culture medium at a density of 5×106 cells/ml. 10 μl of cell suspension were pipetted to each well of the 24-well plate. Four hours after the cells were attached to the yarns, the method added 50 μl fresh culture media to each well and incubated the cell-laden yarns. The media were changed every day. Cell cultivation on Petri dishes was used as a control group. After culturing for 4 h, 3 days, and 7 days, the method stained cells using Alexa Fluor™ 488 phalloidin (Invitrogen, Thermo Fisher Scientific, category number: A12379) according to the manufacturer's instructions and observed cells under a fluorescence microscope (BX53, Olympus®, Japan). The cellular elongation was determined by measuring the aspect ratio of the longest line to the shortest line across the cell shape. The cellular alignment was determined by measuring the angle between the long axis of the cell and the direction of aligned fibers. Quantitative data of cellular elongation and alignment were measured and analyzed using Image J software (NIH, USA).

Sandwich scaffold fabrication: After fabricating the PCL yarns, the method crocheted them into a single chain (FIG. 7A), then extended the single chain into multiple chains (FIG. 7B), using different hook sizes, 2 mm, 4 mm, and 6 mm. After fabricating the textile fabrics, the method fabricated the outer layers of the sandwich scaffold by electrospinning PCL (prepared as described above) on both sides of the multiple chains (FIG. 7C). To optimize the thickness of the outer layers of the sandwich scaffold, the method used different electrospinning duration, 10 min, 20 min, and 40 min, in the outer layer fabrication process labeled as Scaffold-10, Scaffold-20, and Scaffold-40. The electrospinning process was carried out in an ambient environment. The electrospinning voltage was set at 10 kV, the flow rate at 35 l/min, and the distance between the nozzle tip to the textile fabric surface at 150 mm.

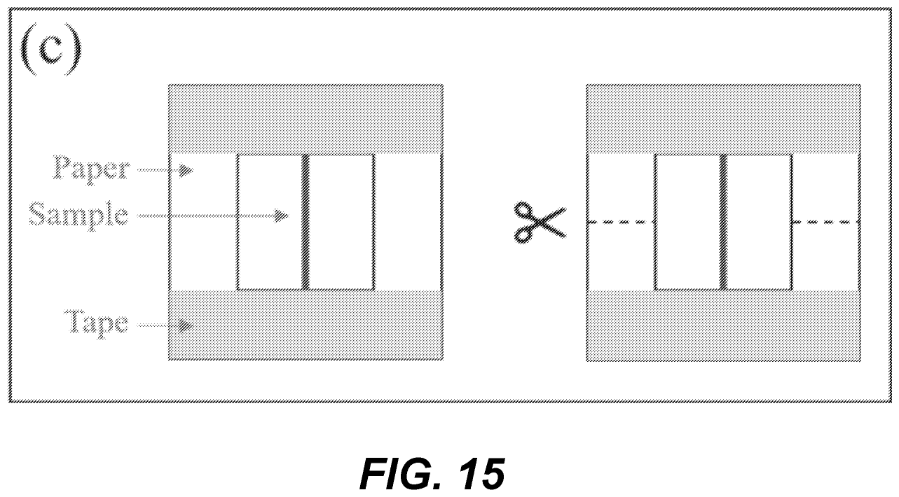

Characterization of sandwich scaffolds: To test the mechanical properties of the sandwich scaffold and its components, the method fixed samples in an aperture card according to ASTM C1557 (FIG. 15) (ASTM, 2014), and then conducted tensile testing in a universal testing machine (ESM303, MARK-10, USA) at a rate of 20 mm/s until failure. The initial length of samples was determined by the aperture card and set at 20 mm. To measure the cross-section areas of samples, the study observed the cross-section of samples under SEM and measured the cross-section area using Image J software. The maximum stress and the maximum strain were the maximum stress value before failure and the corresponding strain value, respectively. The elastic modulus was defined as the slop of the linear portion of the stress-strain curve before the yield point. Resistance to deformation and restoration of shape after a period of distortion are desirable qualities of the skin (Ryan, 1995), so the resilience of sandwich scaffolds was characterized by calculating the area underneath the stress-strain curve before the yield point. To test the biological performance of sandwich scaffolds, the study seeded cells on sandwich scaffolds and cultured cell-laden scaffolds. Before seeding cells, the study placed sandwich scaffolds in Petri dishes and sterilized the scaffolds by exposing them to the ultraviolet light for 30 min on both sides. Passage 15 HUVECs were harvested and suspended in culture medium at a density of 5×106 cells/ml. 3 ml of cell suspension were pipetted to the upper surface of each sandwich scaffold. Four hours after the cells were attached, the study flipped the scaffolds and added 3 ml cell suspension to the upper surface of each sandwich scaffold. Four hours after the cells were attached, the method added 4 ml of fresh culture media to each Petri dish and incubated the cell-laden scaffolds. The media were changed every day. To observe cell morphology under SEM, the method fixed and dehydrated cells after culturing the cell-laden scaffolds for 3 and 7 days. The cell-laden scaffolds were immersed in 2.5% GA for 30 min and then dehydrated in a series of aqueous ethanol solutions of 30%, 50%, 70%, 90%, and 100% for 10 min, respectively. The samples were then gold-sputtered and characterized by SEM.

Statistics: All experiments were performed with ten replicates for each sample. Statistical comparisons were performed using Student's t-test. A significance level of 0.05 was applied to determine significant differences. The alignment degree and crystalline degree of fibers, the cellular elongation, and the cellular alignment were presented as mean±standard deviation. JMP Pro (Version 14, SAS Institute, Inc. Cary, NC) was used for the Design of Experiments (DOE) in the crystalline degree and mechanical strength investigation.

### Results and Discussion

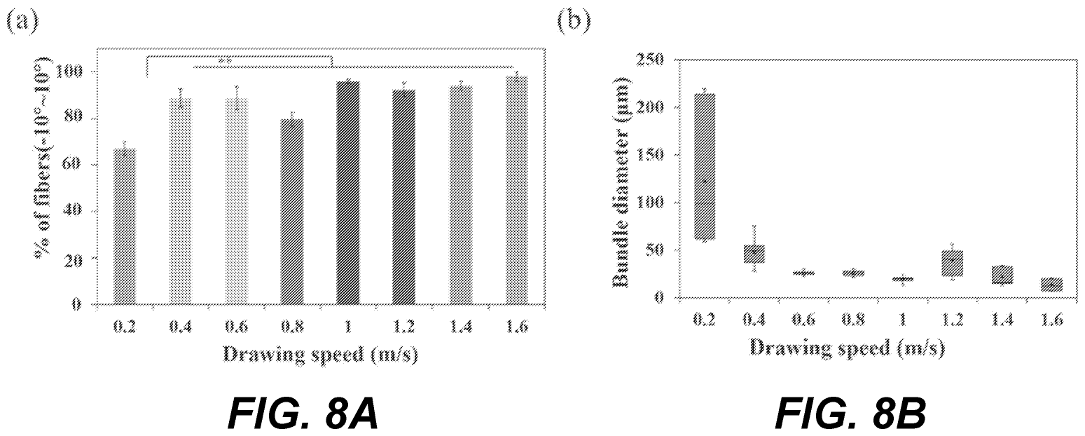

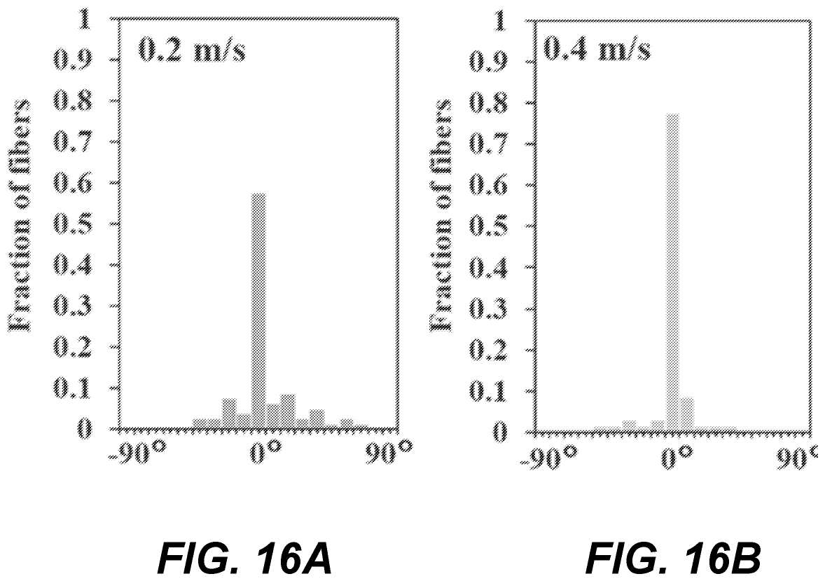

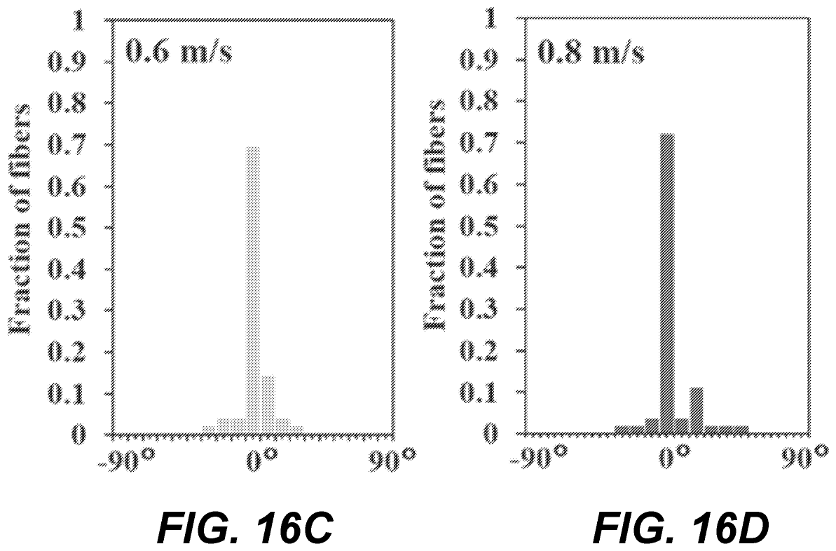

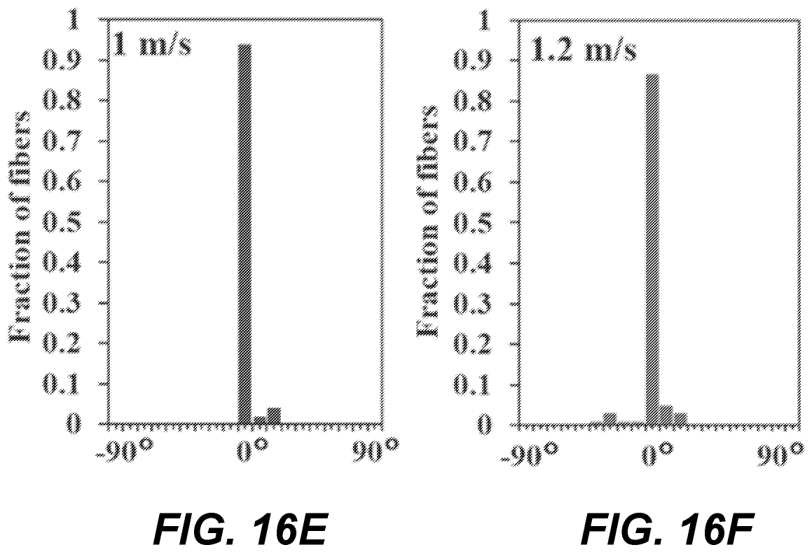

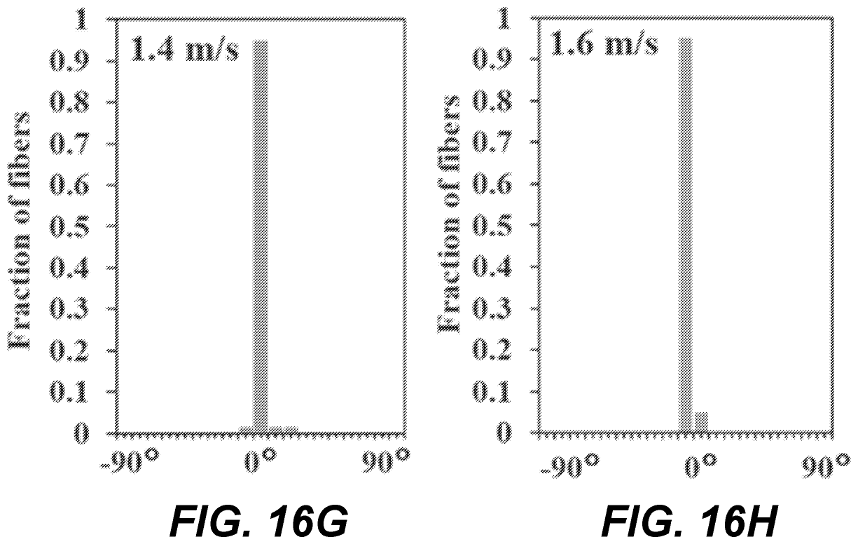

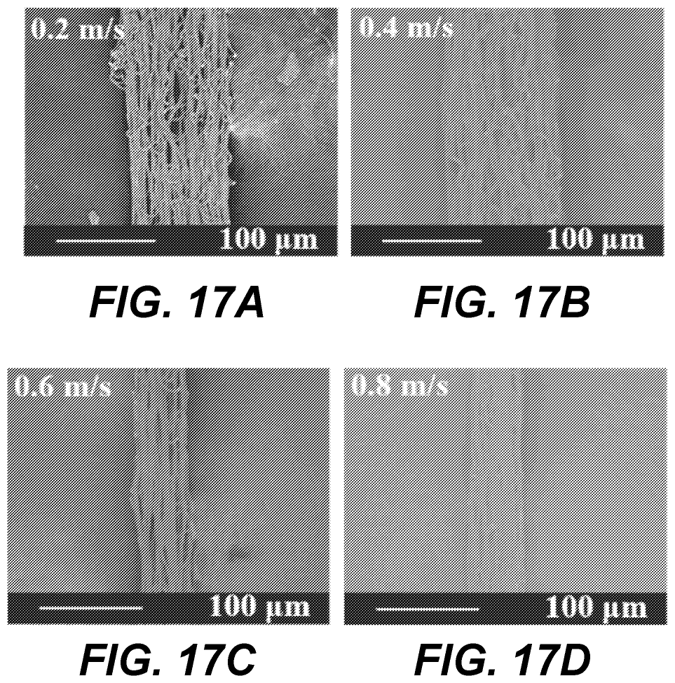

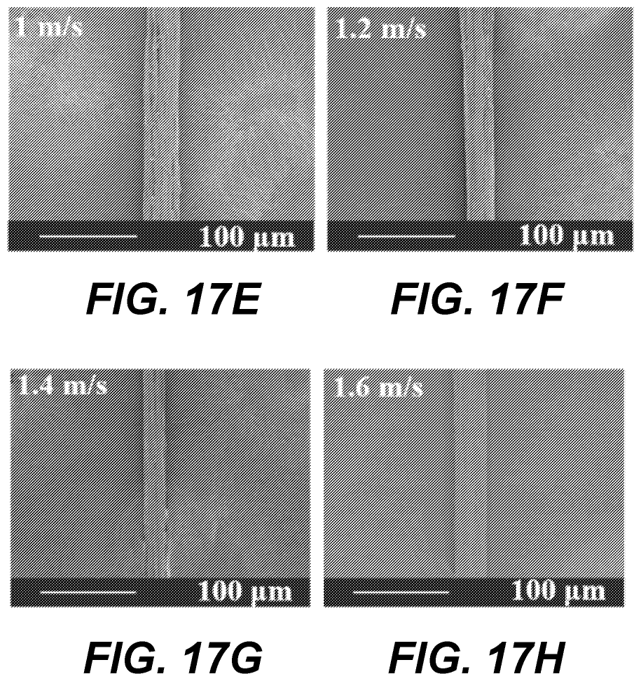

Yarn morphologies: The orientation of fibers and the diameter of bundles were both regulated by the drawing speed of the take-up roller (FIG. 8A and FIG. 8B). The percentage of fibers aligned within ±10° in a bundle increased from 67±3% to 88±4% when the drawing speed increased from 0.2 m/s to 0.4 m/s (FIG. 8A, FIG. 16A-16H). At low drawing speeds (0.2-0.4 m/s), the bundle diameter varied greatly as fibers got tangled in the bundle (FIG. 8B, FIGS. 17A-17H). At moderate drawing speeds (0.6-1 m/s), the bundle diameter became more consistent because all fibers aligned tightly in the bundle. At high drawing speeds (1.2-1.6 m/s), the bundle diameter diverged again from run to run as some fibers deformed or broke. Thus, moderate drawing speeds, 0.6-1 m/s, will be used to fabricate yarns in the following experiments.

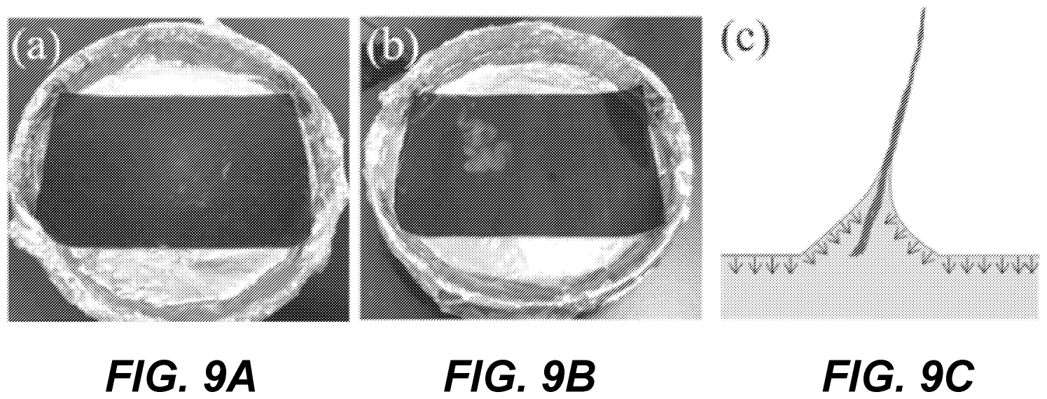

Investigation of the bath liquid composition effect: To investigate the effect of bath liquid composition on the wet electrospinning process, the study used ethanol/water mixtures with different ratios as the liquid baths. The study found that fibers were organized into a web-like structure floating on the surface of the pure water bath (FIG. 9A) but a thread-like structure submerging in the pure ethanol bath (FIG. 9B). Fibers broke apart easily when being drawn from the pure water bath, while formed into a continuous bundle when being drawn from the pure ethanol bath. It is contemplated that different surface tension of water (72.8 mN/m) and ethanol (22.1 mN/m) is the primary reason for producing fibers with such different properties. Because of the high surface tension of water, fibers cannot break the hydrogen bond on the water surface easily and will float on the water bath. During the drawing process (FIG. 9C), the large adhesive force between the water molecules and the fibers makes fibers easy to break. When the ethanol/water ratio was higher than 0.5 (surface tension was lower than 28.52 mN/m) (Vazquez et al., 1995), the study was able to get a continuous bundle.

Investigation of the crystalline degree of fibers: To investigate the effect of different process parameters on the crystalline degree of fibers, the study applied the Design of Experiments (DOE) method using the statistical software JMP Pro (Version 14, SAS Institute, Inc. Cary, NC). Combinations of ethanol concentration (0-1), drawing speed (0, 0.5, 1 m/s), and the disallowed combination (ethanol concentration<0.5 & Drawing speed>0) were used to generate a response surface using the following regression model:

$$\\ \begin{matrix}Y = I + {α}_{1} {X}_{1} + {α}_{2} {X}_{2} + {α}_{12} {X}_{1} {X}_{2} + {α}_{11} {X}_{1}^{{}_{} 2} + {α}_{22} {X}_{2}^{{}_{} 2} & ( 1 )\end{matrix}$$

where Y is the crystalline degree of fibers, I is the intercept, αi(j) is the factor coefficient, X1 is the drawing speed, and X2 is the ethanol concentration. Table 1 shows the experimental groups and corresponding results of the crystalline degree, and Table 2 shows the factor coefficients in Equation (1).

TABLE 1
Experimental group and results in crystalline degree study.

| | Drawing | Alcohol | Crystalline |
|---|---|---|---|
| Run | speed (m/s) | concentration | degree |
| 1 | 0 | 0 | 0.2113 |
| 2 | 0 | 0.7 | 0.2747 |
| 3 | 1 | 0.5 | 0.2016 |
| 4 | 1 | 0.9 | 0.2522 |
| 5 | 1 | 0.5 | 0.2016 |
| 6 | 1 | 0.9 | 0.2522 |
| 7 | 0 | 0.3 | 0.3366 |
| 8 | 0 | 1 | 0.3090 |
| 9 | 0.5 | 0.5 | 0.2006 |
| 10 | 0.5 | 0.5 | 0.2006 |

TABLE 2
Factor coefficients in Equation (1).

| | Significance | |
|---|---|---|
| Term | level | Coefficient |
| Intercept | 0.0010 | 0.2001 |
| Drawing speed | 0.0372 | −0.0564 |
| Alcohol concentration | 0.0707 | 0.0691 |
| Drawing speed * Drawing speed | 0.1265 | 0.0573 |
| Alcohol concentration * Alcohol | 0.2415 | −0.0542 |
| concentration | | |
| Drawing speed * Alcohol concentration | 0.2535 | 0.0376 |

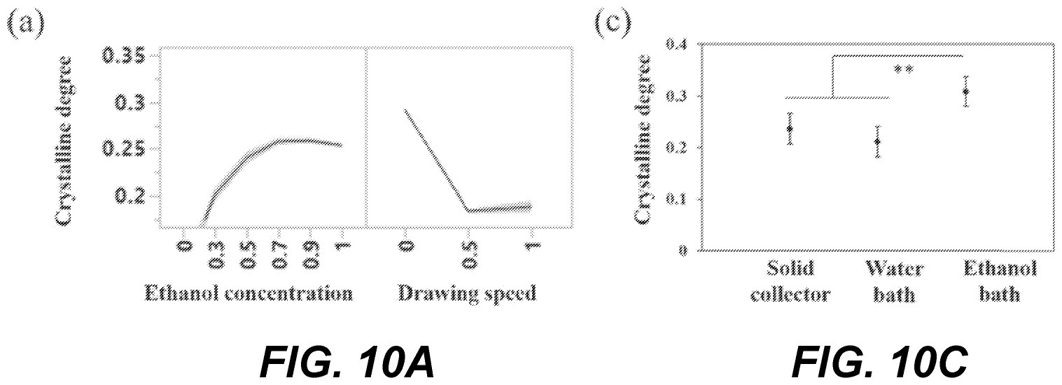

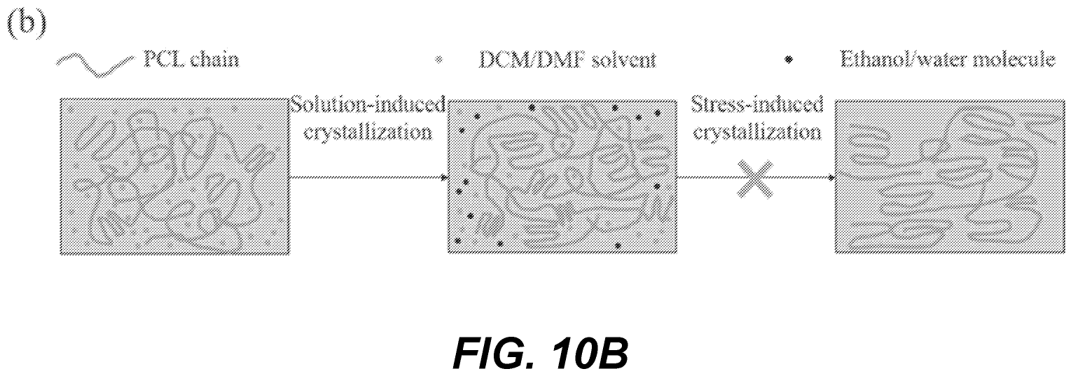

As shown in the model plot (FIG. 10A), increasing ethanol concentration increased the crystalline degree of PCL fibers. When PCL fibers were deposited in the liquid bath, solvent extraction from the fibers increased the interaction between PCL molecular chains to activate a solution-induced crystallization (FIG. 10B). The solution-induced crystallization effect was enhanced when the fibers changed from floating on to submerging in the bath as the ethanol/water ratio increased. The study also found no significant difference in crystallinity between the PCL fibers collected with the water bath collector and the solid collector (FIG. 10C), suggesting that fibers floating on the bath likely did not lead to solution-induced crystallization.

Previous applications of wet electrospun yarns in tissue engineering have only used a specific ratio of ethanol/water mixtures (Khil et al., 2005; Wang et al., 2015a; Wu et al., 2017), but never investigated the effect of bath compositions on the property of wet electrospun yarns. In this study, it is demonstrated that the bath composition affected the properties of wet electrospun yarns, providing the possibility to post-process electrospun fibers by varying bath compositions. Functional molecules can also be added to the bath to functionalize electrospun fibers.

Another result from the model plot was that the presence of the drawing process decreased the crystalline degree (FIG. 10A). This result demonstrates that stress-induced crystallization does not occur during the drawing process in wet electrospinning (FIG. 10B). It is contemplated that the drawing force during the wet electrospinning is too mild to lead to stress-induced crystallization. Meanwhile, the presence of the drawing process decreased the time for solution-induced crystallization, leading to a decrease in the crystalline degree of fibers.

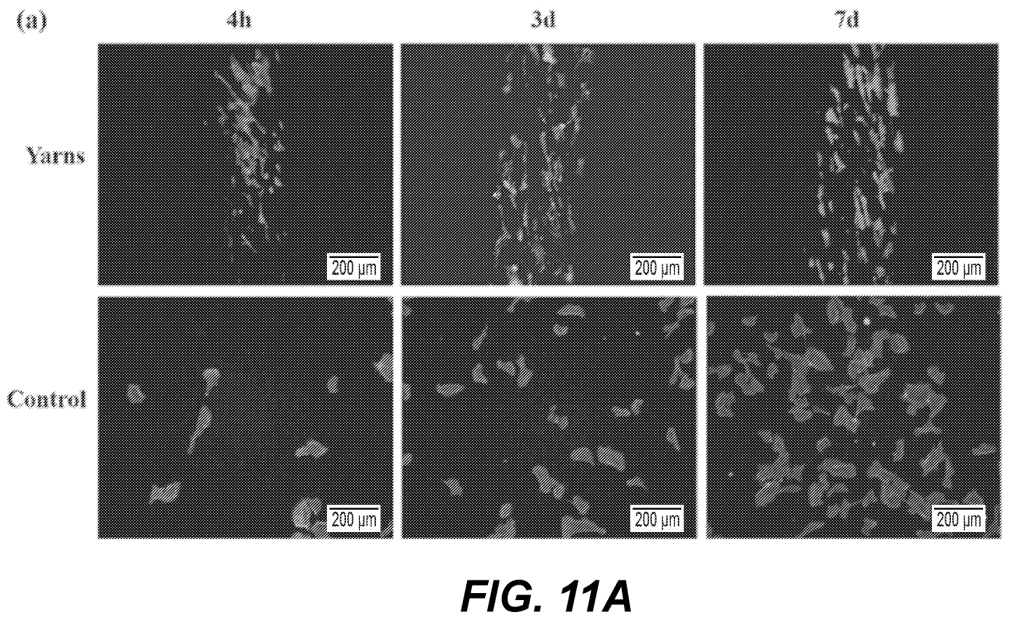

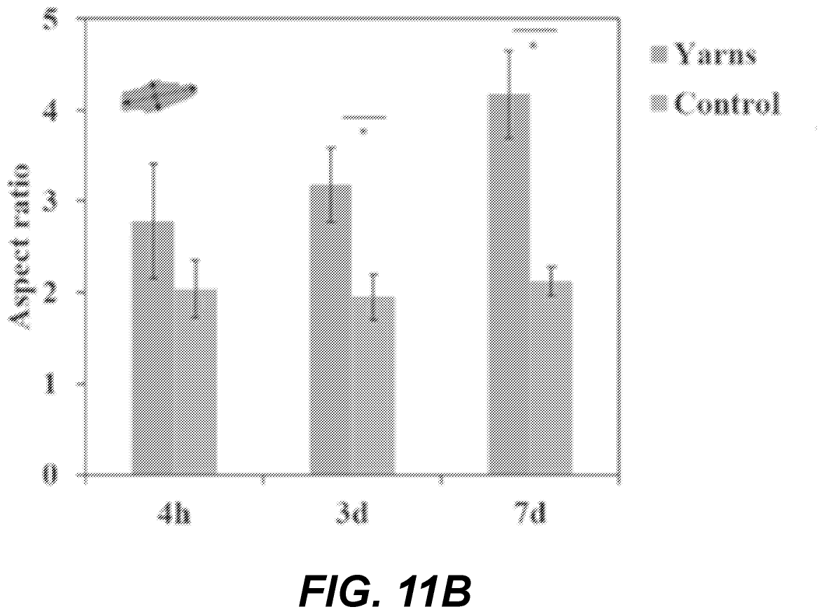

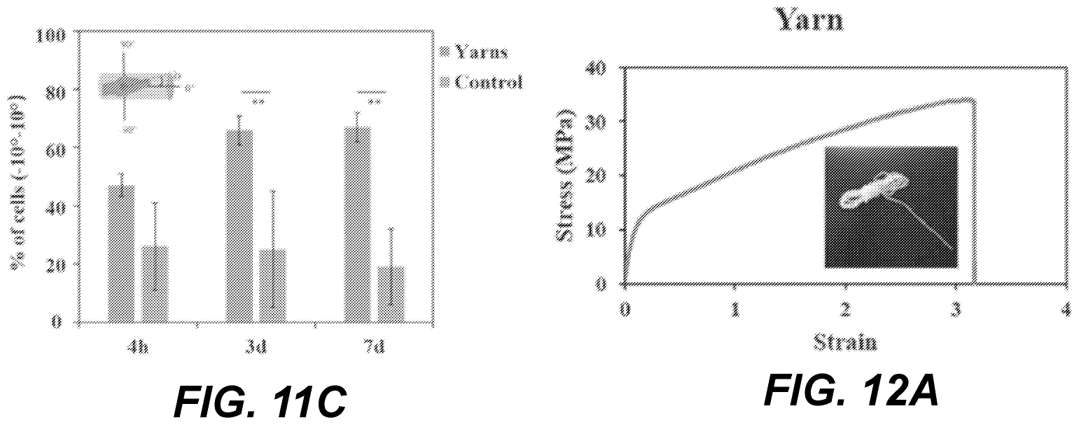

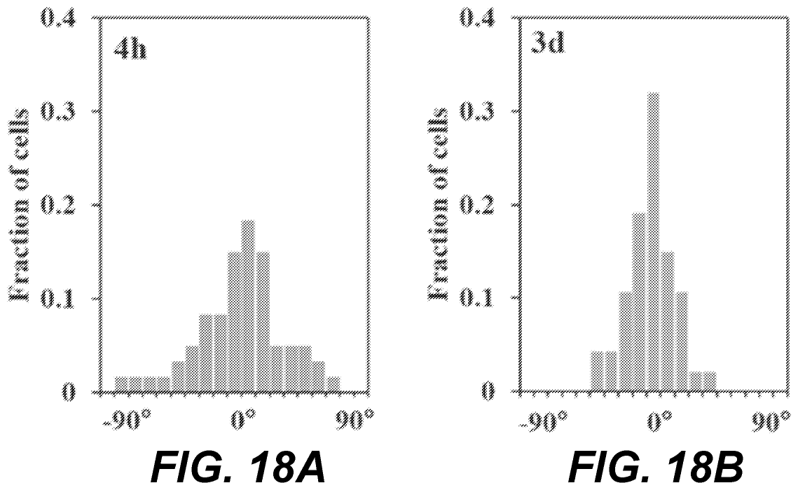

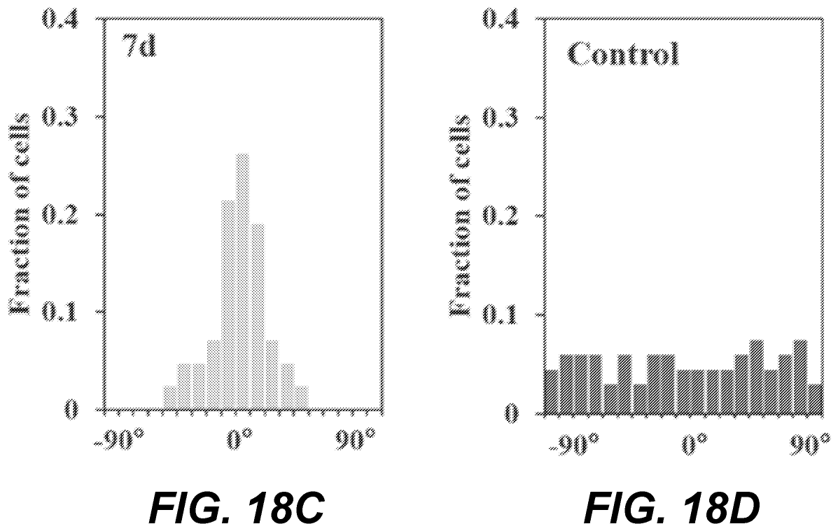

Cell growth on wet electrospun yarns: To investigate the biological properties of wet electrospun yarns, cellular elongation and cellular alignment were analyzed after cell cultivation on yarns for 4 h, 3 days, and 7 days. Fluorescent images (FIG. 11A) showed that cells grown on yarns performed the elongation and alignment behavior, while cells grown on Petri dishes showed the normal random morphology. Quantitative analysis of cellular elongation (FIG. 11B) showed that the aspect ratios of cells grown on yarns were significantly higher than those of cells grown on Petri dishes after culturing for 3 days. Furthermore, the aspect ratio of cells after culturing for 4 h was 2.78±0.63, while the aspect ratios increased to 3.18±0.41 and 4.17±0.48 after culturing for 3 days and 7 days. On the other hand, the quantitative analysis of cellular alignment was evaluated by measuring the angle between the long axis of the cells and the direction of aligned fibers (FIG. 18A-18D), and the cells that aligned within ±10° were considered to be aligned (FIG. 11C). The results showed that cells on yarns exhibited significantly higher alignment morphology than those on Petri dishes. Furthermore, the percentage of cells aligned within ±10° increased from 47±4% when culturing for 4 h to 66±5% when culturing for 3 days. These data demonstrated the good biocompatibility of wet electrospun PCL yarns and their ability to guide cellular elongation and alignment during cell cultivation.

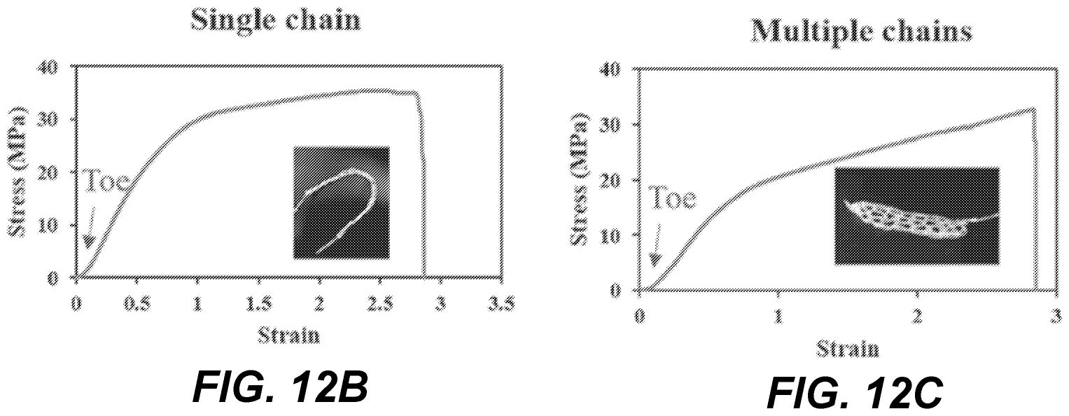

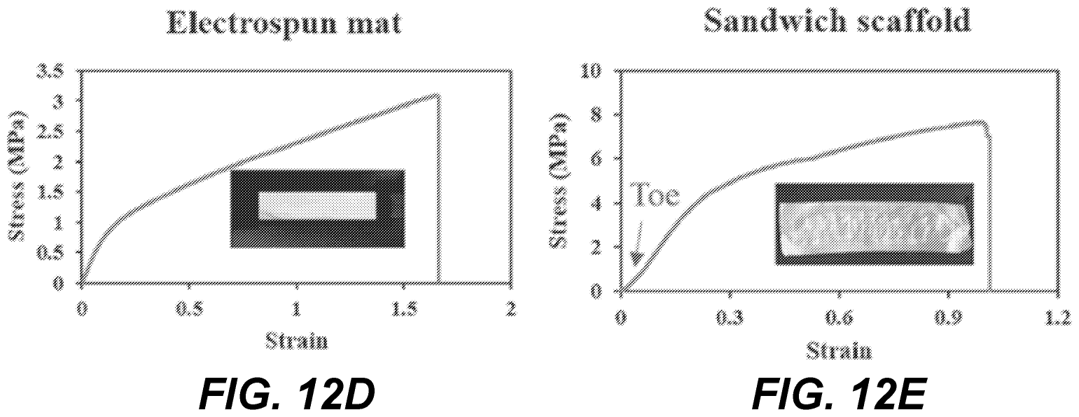

Mechanical properties of sandwich scaffolds: The sandwich scaffold has similar physical properties to human skin. The middle layer of the human skin, the dermis, is mainly comprised of interconnected collagen fibers and provides mechanical support for the skin (FIG. 5) (Mathes et al., 2014). The textile fabric in the middle layer of the sandwich scaffold has hierarchical fiber-bundle-yarn organizations and interconnected porous structures similar to the collagen fibers in human skin. The tensile testing results (FIGS. 12A-12E) showed that although the yarn itself did not have strain-stiffening property, the single chain and the multiple chains fabricated from the yarn exhibited strain-stiffening behavior, resulting in a strain-stiffening sandwich scaffold.

To investigate the effect of different process parameters on the mechanical properties of the sandwich scaffold, the study used the DOE method and considered combinations of ethanol concentration (0.5-1), drawing speed (0.6-1 m/s), and hook size (2, 4, 6 mm). The generated regression model was:

$$\\ \begin{matrix}Y = I + {α}_{1} {X}_{1} + {α}_{2} {X}_{2} + {α}_{3} {X}_{3} + {α}_{12} {X}_{1} {X}_{2} + {α}_{13} {X}_{1} {X}_{3} + {α}_{23} {X}_{2} {X}_{3} & ( 2 )\end{matrix}$$

where Y is the maximum stress, maximum strain, elastic modulus, or resilience of the sandwich scaffold, I is the intercept, αi(j) is the factor coefficient, X1 is the drawing speed, X2 is the ethanol concentration, and X3 is the hook size. Table 3 shows the experimental groups and corresponding results of mechanical properties, and Tables 4-7 show the factor coefficients in Equation (2). FIG. S5 shows residual by predicted plots. FIG. S6 shows residual normal quantile plots.

TABLE 3
Experimental group and results in mechanical properties study.

| | Drawing | | Hook | Maximum | | | |
|---|---|---|---|---|---|---|---|
| | speed | Alcohol | size | stress | Maximum | Modulus | Resilience |
| Run | (m/s) | concentration | (mm) | (MPa) | strain | (MPa) | (MPa) |
| 1 | 0.6 | 1 | 6 | 6.3002 | 0.6550 | 27.1888 | 0.0566 |
| 2 | 0.6 | 1 | 2 | 5.7718 | 1.8595 | 4.4367 | 0.5164 |
| 3 | 1 | 1 | 6 | 9.8452 | 1.3245 | 11.7012 | 1.4627 |
| 4 | 1 | 0.5 | 6 | 9.3027 | 2.0640 | 5.3668 | 2.9556 |
| 5 | 1 | 0.75 | 4 | 6.6870 | 0.8310 | 8.1888 | 2.6041 |
| 6 | 1 | 0.5 | 2 | 8.0818 | 1.4315 | 5.6457 | 5.7845 |
| 7 | 0.8 | 0.5 | 4 | 12.3769 | 1.9385 | 9.0443 | 6.1060 |
| 8 | 0.8 | 0.75 | 2 | 4.7757 | 0.6710 | 7.2432 | 1.5538 |
| 9 | 1 | 1 | 2 | 8.4935 | 1.2795 | 7.4173 | 4.5777 |
| 10 | 0.8 | 0.75 | 4 | 9.5005 | 1.8490 | 6.6220 | 6.0209 |
| 11 | 0.8 | 0.75 | 6 | 9.5262 | 1.8240 | 5.2227 | 8.6879 |
| 12 | 0.8 | 1 | 4 | 4.9516 | 1.2340 | 4.0126 | 3.0551 |
| 13 | 0.6 | 0.5 | 2 | 9.2523 | 1.2265 | 8.3630 | 4.5010 |
| 14 | 0.6 | 0.5 | 6 | 14.4211 | 2.3385 | 7.8815 | 10.5732 |
| 15 | 0.6 | 0.75 | 4 | 6.9400 | 1.4305 | 5.1022 | 4.6666 |

TABLE 4
Factor coefficients in Equation (2) when Y is
the maximum stress of the sandwich scaffold.

| | Significance | |
|---|---|---|
| Term | level | Coefficient |
| Intercept | 0.0001 | 8.4151 |
| Alcohol concentration | 0.0139 | −1.8073 |
| Drawing speed * Alcohol concentration | 0.0409 | 1.5694 |
| Hook size | 0.0539 | 1.3020 |
| Alcohol concentration * Hook size | 0.4074 | 0.1738 |
| Drawing speed * Hook size | 0.5614 | −0.3906 |
| Drawing speed | 0.9631 | −0.0273 |

TABLE 5
Factor coefficients in Equation (2) when Y is
the maximum strain of the sandwich scaffold.

| | Significance | |
|---|---|---|
| Term | level | Coefficient |
| Intercept | 0.0001 | 1.4638 |
| Alcohol concentration * Hook size | 0.0433 | −0.3630 |
| Alcohol concentration | 0.0863 | −0.2647 |
| Hook size | 0.2351 | 0.1738 |
| Drawing speed * Hook size | 0.5425 | 0.0963 |
| Drawing speed | 0.6798 | −0.0580 |
| Drawing speed * Alcohol concentration | 0.8988 | 0.0199 |

TABLE 6
Factor coefficients in Equation (2) when Y
is the modulus of the sandwich scaffold.

| | Significance | |
|---|---|---|
| Term | level | Coefficient |
| Intercept | 0.0002 | 8.2291 |
| Alcohol concentration * Hook size | 0.0740 | 3.4745 |
| Hook size | 0.1476 | 2.4255 |
| Drawing speed * Hook size | 0.2140 | −2.2832 |
| Alcohol concentration | 0.2573 | 1.8455 |
| Drawing speed | 0.3612 | −1.4652 |
| Drawing speed * Alcohol concentration | 0.6055 | −0.9093 |

TABLE 7
Factor coefficients in Equation (2) when Y
is the resilience of the sandwich scaffold.

| | Significance | |
|---|---|---|
| Term | level | Coefficient |
| Intercept | 0.0001 | 4.2081 |
| Alcohol concentration | 0.0154 | −2.0252 |
| Drawing speed * Alcohol concentration | 0.0808 | 1.4752 |
| Drawing speed * Hook size | 0.0861 | −1.4445 |
| Alcohol concentration * Hook size | 0.2817 | −0.8523 |
| Hook size | 0.3331 | 0.6802 |
| Drawing speed | 0.6691 | −0.2929 |

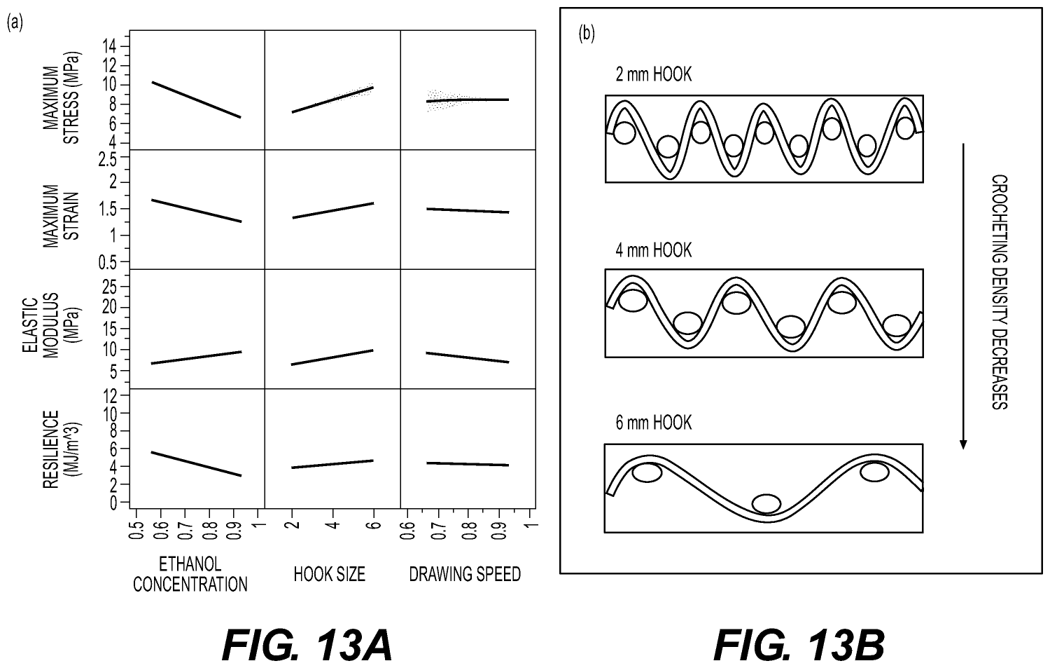

As shown in the model plot (FIG. 13A), increasing ethanol concentration decreased the resilience of the sandwich scaffold significantly. Increasing ethanol concentration can increase the crystalline degree of the fibers that leads to a scaffold with higher rigidity and lower resilience (Djafari Petroudy, 2017). Another result from the model plot was that increasing the hook size increased the maximum strain (FIG. 13A). The reason is that increasing the hook size results in fewer crossover points and failure nuclei (Khondker et al., 2001).

Based on the above investigation, the method can adjust the mechanical properties of the sandwich scaffold to match those of human skin. As the model predicts, the sandwich scaffold maximum stress can be adjusted from 3.74 to 11.82 MPa, the maximum strain from 0.16 to 2.37, and the elastic modulus from 2.10 to 18.05 MPa, all falling within the ranges of human skin (Table 8). Mechanical properties of skin scaffolds reported in other studies were either outside the ranges of human skin or not mentioned (Chen et al., 2000; Han et al., 2014; Ng and Hutmacher, 2006; Wang et al., 2012b, 2015b; Wu et al., 2019). However, the toe region of sandwich scaffolds is smaller (strain: 0.05-0.16) compared with that of human skin (0.3-0.6) (Daly and Odland, 1979; Joodaki and Panzer, 2018). Other materials and more curved textile patterns will be used to increase the toe region of the scaffold in our future studies.

TABLE 8
Comparison of mechanical strength of human
skin and reported skin scaffolds.

| [symbol] | [symbol] | [symbol] | [symbol] | [symbol] | [symbol] |
|---|---|---|---|---|---|
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |

[symbol]

indicates data missing or illegible when filed

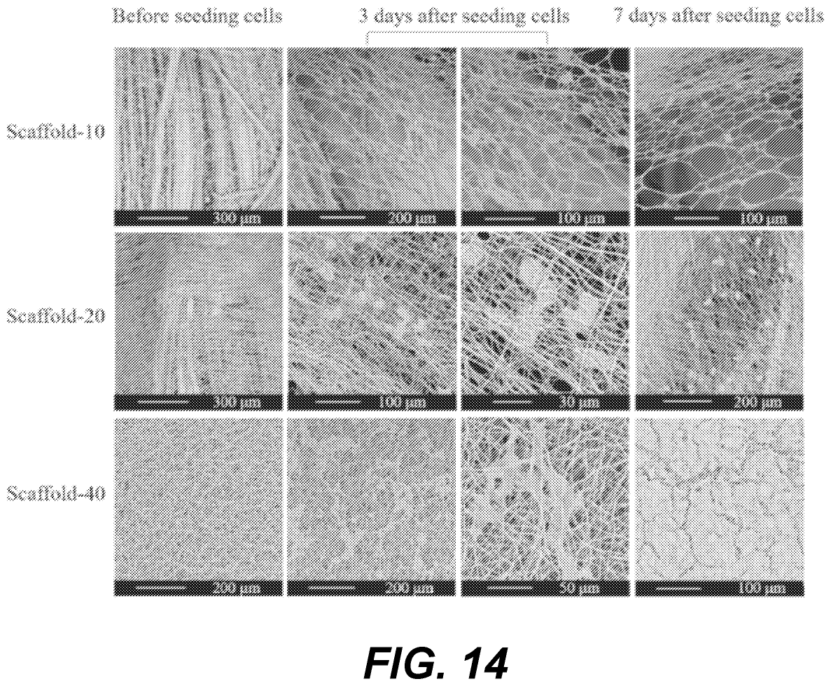

Cell growth on sandwich scaffolds: To investigate the biological properties of sandwich scaffolds, the study observed the morphologies of cells grown on Scaffold-10, Scaffold-20, and Scaffold-40. After culturing for 3 days (FIG. 14), some cells on Scaffold-10 and Scaffold-20 grew under the electrospun fibers, a sign of infiltration, while all the cells on Scaffold-40 grew above the electrospun fibers. After culturing for 7 days, a layer of cell membrane formed on the surface of Scaffold-40, making cell infiltration more difficult, while the relatively sparser electrospun fibers of Scaffold-10 and Scaffold-20 enabled cells to penetrate. However, the electrospun fibers of Scaffold-10 were so sparse that cells have difficulty in spreading their pseudopodia to surrounding areas. Thus, Scaffold-20 is the optimal group that can support cell infiltration and proliferation.

Scaffolds fabricated by electrospinning are widely used in tissue engineering because of their fibrous structures and high flexibility. However, scaffolds fabricated by electrospinning are limited to 2-dimensional (2D) structures with low mechanical properties and can only be applied to repair superficial or epidermis-level skin wounds. For full-thickness skin wounds, scaffolds with 3-dimensional (3D) structures and sufficient mechanical properties are necessary for skin regeneration. In this study, a textile fabric was study as the middle layer to increase the thickness and mechanical properties of electrospun scaffolds. The electrospun fibers in outer layers provide an ideal niche for cell attachment. By optimizing the thickness of the outer layers, the study can obtain a scaffold that also supports cell proliferation and infiltration. Moreover, the textile fabric in the middle layer, featuring interconnected porous structures, enables the transportation of nutrients and oxygen and removal of metabolic waste during cell growth. The textile fabric can also be manufactured from different materials from the outer layers to mimic the gradients of various growth factors, cytokines, and enzymes in vivo to promote optimal restoration and regeneration of full-thickness skin wounds.

In summary, a textile-based sandwich scaffold using wet electrospun yarns has been developed that can mimic the physical, mechanical, and biological properties of human skin. For the first time, the effect of bath compositions on properties of wet electrospun yarns was analyzed to provide guidelines on how to control the morphologies and mechanical properties of wet electrospun yarns. These wet electrospun yarns, highly customizable with a hierarchical organization, have the ability to induce cellular elongation and cellular alignment. In this study, the crochet method was used to fabricate textile fabrics with interconnected porous structures similar to the collagen fibers in human skin. These textile fabrics were embedded inside two electrospun mats to create a sandwich scaffold. The scaffold exhibited both the strain-stiffening behavior of the textile fabric and provided an ideal niche for cell attachment on the electrospun mats. After optimization, the textile-based sandwich scaffold not only has similar physical, mechanical properties to human skin but also supports initial cell attachment and long-term cell proliferation and infiltration. These features make the scaffold highly desirable for applications in tissue engineering.

### REFERENCES

Akbari, M., et al., Adv. Healthc. Mater. 5, 751-766 (2016).
ASTM, 2014. Standard Test Method for Tensile Properties of Polymer Matrix Composite Materials D3039, vol. 15. ASTM Stand., pp. 1-13
Chen, G., et al., Chem. Commun. 1505-1506, (2000).
Daly, C. H., et al., J. Invest. Dermatol. 73, 84-87, (1979).
Djafari Petroudy, S. R., 2017. Physical and mechanical properties of natural fibers, in: advanced high strength natural fibre composites in construction, 59-83.
Freeman, J. W., et al., J. Biomech. 40, 2029-2036 (2007).
Gallagher, A. J., et al., 2012. Dynamic tensile properties of human skin, in: 2012 IRCOBI conference proceedings—international research Council on the biomechanics of injury.
Gupta, B., et al., J. Appl. Polym. Sci. 123, 1944-1950 (2012).
Han, F., et al., Acta Biomater. 10, 921-930 (2014).
JANSEN, L. H., et al., Dermatol. 117, 65-83 (1958).
Jaspers, M., et al., Nat. Commun. 5, 5808, (2014).
Jaspers, M., et al., Nat. Commun. 8, 15478 (2017).
Joodaki, H., et al., 2018. Skin mechanical properties and modeling: a review. Proc. Inst. Mech. Eng. Part H J. Eng. Med.
Khil, M. S., et al., J. Biomed. Mater. Res. B Appl. Biomater. 72, 117-124, (2005).
Khondker, O. A., et al., J. Compos. Mater. 35, 489-508 (2001).
Mathes, S. H., et al., “The use of skin models in drug development.” Adv. Drug Deliv. Rev. (2014).
Maziz, A., et al., “Knitting and weaving artificial muscles.” Sci. Adv. 3, e1600327 (2017).
Mironov, V., et al., Biomaterials 30, 2164-2174 (2009).
Motte, S., et al., Biopolymers 99, 35-46 (2013).
Ng, K. W., et al., Biomaterials 27, 4591-4598 (2006).
Ni Annaidh, J. Mech. Behav. Biomed. Mater. 5, 139-148 (2012).
Pedde, R. D., et al., “Emerging biofabrication strategies for engineering complex tissue constructs.” Adv. Mater. (2017).
Piechocka, I. K., et al., Biophys. J. 98, 2281-2289 (2010).
Ryan, T. J., et al., Clin. Dermatol. 13, 429-432 (1995).
Stark, H. L., et al., Br. J. Plast. Surg. 30, 105-114 (1977).
Tamayol, A., et al., “Fiber-based tissue engineering: progress, challenges, and opportunities.” Biotechnol. Adv. (2013).
Vazquez, G., et al., J. Chem. Eng. Data 40, 611-614 (1995).
Wang, L., et al., ACS Nano 9, 9167-9179 (2015a).
Wang, K., et al., Addit. Manuf 12, 31-37 (2016).
Wang, Z., et al., ACS Nano 9, 271-278 (2015b).
Wang, X., J. Mech. Behav. Biomed. Mater. 8, 204-215 (2012b).
Wang, J. H. C., et al., J. Hand Ther. 25, 133-141 (2012a).
Wu, C., et al., ACS Nano 13, 10727-10736 (2019).
Wu, Y, ACS Nano 11, 5646-5659 (2017).
Zhalmuratova, ACS Appl. Mater. Interfaces 11, 33323-33335 (2019).

### Example 5: Using Wet Electrospun PCL/Gelatin/CNT Yarns to Fabricate Textile-Based Scaffolds for Vascular Tissue Engineering

A study was performed that fabricated PCL/gelatin/CNT composite yarns to construct scaffolds, e.g., for treating CVD. The impacts of different ratios of PCL/gelatin and different concentrations of CNTs on the properties of PCL/gelatin/CNT yarns were analyzed and mapped out to provide guidelines on how to control the mechanical and biological properties of yarns. The inclusion of CNTs enhanced the elongation and alignment of ECs on yarns. To demonstrate the application, PCL/gelatin/CNT yarns were used to fabricate a textile-based scaffold for vascular tissue engineering. The scaffold shares similar structure and mechanical properties to the native vessels. ECs cultured in the lumen of the scaffold proliferated and exhibited an alignment morphology. In all, the study demonstrates that the polymer/CNT composite yarns fabricated by wet electrospinning are excellent candidates for constructing vascular scaffolds. This novel method addresses the limitations of conventional methods to incorporate CNTs in polymers, facilitating the wide application of CNTs in tissue engineering. While the focus of the study was on CNTs, the method can be applied to incorporate other functional materials or biomolecules in biopolymers.

Incorporating conductive materials in scaffolds has shown advantages in regulating adhesion, mitigation, and proliferation of electroactive cells for tissue engineering applications. Among various conductive materials, carbon nanotubes (CNTs) have shown great promises in tissue engineering because of their good mechanical properties. However, the broad application of CNTs in tissue engineering is limited by current methods to incorporate CNTs in polymers that require miscible solvents to dissolve CNTs and polymers or CNT surface modification. These methods either limit polymer selections or adversely affect the properties of polymer/CNT composites. Here, polymer/CNT composite yarns are fabricated by electrospinning polycaprolactone/gelatin into a bath of CNT dispersion and extracting electrospun fibers out of the bath. The concentration of CNTs in the bath affects the thermal and mechanical properties and the yarns' degradation behavior. In vitro biological test results show that within a limited range of CNT concentrations in the bath, the yarns exhibit good biocompatibility and the ability to guide cell elongation and alignment. A vascular scaffold was designed and fabricated by knitting the yarns into a textile fabric and combining the textile fabric with gelatin. The scaffold has similar mechanical properties to native vessels and supports cell proliferation. This work demonstrates that the wet electrospun polymer/CNT yarns are good candidates for constructing vascular scaffolds a d provides a novel method to incorporate CNTs or other functional materials into biopolymers for tissue engineering applications.

Cardiovascular diseases (CVDs) are the leading cause of death both globally and in the U.S., with one person dying every 36 s in the United States from CVD [1′, 2′]. Many of the CVD patients may end up requiring transplantation [3′]. However, transplantation has a high risk of rejection, and there is a great shortage of available organ donors. Vascular tissue engineering has been proposed as a less risky, more accessible alternative option to repair, regenerate, and improve damaged vessels. Tissue engineering uses a combination of cells, scaffolds, and suitable biochemical factors to generate biological substitutes. The scaffold plays a vital role in tissue engineering to provide a suitable platform for cell growth. An ideal scaffold should support cellular activities for easy adhesion, proliferation, and differentiation without any toxic effects on the tissue. It should also be biodegradable to enable the host tissue to regenerate and take its place. The scaffold's mechanical properties should resemble that of the target tissue to provide comparable physical support in vivo [4′]. Selecting the proper biomaterials is imperative to fabricate the right scaffold that meets these requirements.

Commonly used biomaterials in tissue engineering include both natural materials (e.g., alginate, collagen, gelatin, and chitosan) and synthetic materials [e.g., polycaprolactone (PCL) and polylactic acid] [5′]. Conductive materials [e.g., polypyrrole, polyaniline, and carbon nanotubes (CNTs)] are a part of a new generation of synthetic biomaterials that allow the direct delivery of electrical, electrochemical, and electromechanical stimuli to cells [6′-9′]. CNTs have attracted attention in tissue engineering because of their extraordinary mechanical and electrical properties [10′-11′]. Although no consensus has been achieved on the biocompatibility of CNTs [12′-15′], many studies have revealed that the incorporation of CNTs in other biomaterials can improve scaffolds' physical properties and facilitate the attachment, proliferation, and differentiation of cells [16′-18′]. For example, Liang et al. produced a stable silk sericin-CNT ink through noncovalent interactions to improve the biocompatibility of CNTs.¹⁹′ Shin et al. produced a CNT-GelMA photo-cross-linkable scaffold to improve cardiac cell adhesion, organization, and cell-cell coupling [20′]. Kharaziha et al. demonstrated that incorporating CNTs in a poly(glycerol sebacate)/gelatin scaffold improved a cardiac construct's spontaneous and synchronous beating behavior [21′].

Although CNTs have great advantages in tissue engineering, incorporating CNTs in polymers remains an issue [22′]. Currently, there are two major methods to incorporate CNTs in the polymer matrix. One method is to use miscible solvents to dissolve CNTs and polymers and then mix them by stirring or sonication [23′, 24′]. Only a limited number of polymers can be dissolved under this method. Another method is to use surface-modified CNTs with high dispersal ability in the polymer matrix [3′, 25′, 26′]. This method may adversely affect the properties of polymer/CNT composites by altering the polymer/CNT interface.²⁷′ Moreover, surface modification of CNTs requires the use of highly complicated techniques, including living polymerization,[28′-30′] oxidation of acids [31′, 32′], and nitro oxide-mediated radical polymerization (NMRP).^{33′,34}′ Thus, new methods to incorporate CNTs in polymers need to be explored to facilitate the broader application of CNTs in tissue engineering.

Polymer/CNT composite yarns were fabricated by wet electrospinning. Wet electrospinning is a modification of the existing electrospinning technique by replacing solid collectors with liquid collectors. When electrospun fibers are deposited into a chemical bath, components in the bath can be coated on electrospun fibers and affect fibers' properties. Wet electrospinning has been used to load functional molecules onto electrospun scaffolds [35′-38′]. The technique can also be used to fabricate well-aligned fibrous yarns by adding a roller to extract electrospun fibers from the bath that can be later assembled into scaffolds via textile processes [39′-42′].

In this scenario, CNTs were incorporated into polymers by electrospinning polymers into a bath of CNT dispersion to improve polymers' mechanical and biological properties. To evaluate the process, a mixture of PCL and gelatin were electrospun into a bath of CNT/propanol dispersion and then extracted electrospun fibers from the bath to get PCL/gelatin/CNT yarns. PCL is a semi-crystalline polymer that has been widely used in tissue engineering due to its excellent rheological and viscoelastic properties [43′]. However, the hydrophobicity and low degradation rate of PCL can adversely affect cell growth on PCL scaffolds. Blending PCL with natural biomaterials such as gelatin can improve the hydrophilicity and biodegradation rate of the resulting scaffold [44′-46′]. In this study, the effects of different ratios of PCL/gelatin and different concentrations of CNTs were investigated as to the effects on the properties of PCL/gelatin/CNT yarns. This study was used to create a database for customizing the mechanical and biological properties of wet electrospun yarns. The concentration of CNTs in the bath was observed to significantly affect the thermal and mechanical properties and the degradation behavior of the yarns. The evaluation of in vitro cells cultured on the PCL/gelatin/CNT yarns showed an increased cell elongation and alignment along the yarn direction compared with those on yarns without CNTs. The study also demonstrated the applicability of PCL/gelatin/CNT yarns in vascular tissue engineering by knitting the yarns into a textile fabric and combining the textile fabric with gelatin to fabricate a vascular scaffold. Textile techniques, including knitting, weaving, and braiding, have shown great advantages in mimicking hierarchical structures and strain-stiffening properties of human tissues [47′-48′]. The scaffold with the knitted fabric and a hydrogel component had similar mechanical properties to native vessels and supported cell proliferation. This work provides a novel method to fabricate polymer/CNT composite yarns with great potentials in constructing scaffolds for electroactive tissues.

### Materials and Methods

Materials. PCL (Mₙ=80,000, product number: 440744), gelatin (type A, product number: G2500), CNTs (multi-walled, product number: 724769), 2,2,2-trifluoroethanol (TFE, product number: T63002), 2-propanol (product number: 278475), acetic acid (product number: 695092), and glutaraldehyde solution (25%, product number: G6257) were purchased from Sigma-Aldrich. Moo Gloo transglutaminase (TG) was purchased from Modernist Pantry. Endothelial Cells (ECs, category number: C-12200) and the endothelial cell growth medium (category number: C-22010) were purchased from PromoCell.

Trypsin-ethylenediaminetetraacetic acid (EDTA) (0.25%, category number: 25200-072), Alexa Fluor 488 phalloidin (category number: A12379), DAPI (category number: D1306), CellTracker CM-Dil Dye (category number: C7000), and a LIVE/DEAD Viability/Cytotoxicity Kit (category number: L3224) were purchased from Thermo Fisher Scientific.

Yarn Fabrication. To prepare polymer solutions for electrospinning, a PCL/TFE solution (10%, w/w) and gelatin/TFE solution (10%, w/w) was mixed at different weight ratios of 10:0, 9:1, and 8:2 (PCL, P9/G1, and P8/G2, respectively). The method added 20 PL of acetic acid to a 10 g PCL/gelatin mixture to create a homogeneous PCL/gelatin solution. The bath for wet electrospinning was prepared by dispersing CNTs in 2-propanol at different concentrations of 60 mg/L, 120 mg/L, and 180 mg/L (CNT60, CNT120, and CNT180, respectively) under sonication at a 30% amplitude for one h (Q700CA Sonicator, QSONICA). 2-Propanol without CNTs was used for a control group.

To fabricate wet electrospun yarns (FIGS. 6A, 6B), “as-prepared” polymer solutions were loaded in a syringe and electrospun at 40 μL/min and 10 kV. A container filled with “as-prepared” CNT dispersions was placed under the electrospun nozzle, and the distance between the nozzle tip to the bottom of the container was set at 40 mm. A piece of aluminous foil was attached to the container bottom and then grounded the aluminous foil. The bath height in the container was 5 mm. When electrospun fibers were deposited in the bath, a tweezer was used to extract the fibers out of the bath and to form a bundle, and the bundle was then lifted on a take-up roller. The velocity of the take-up roller was 0.6 m/s. The yarn is obtained after collecting the bundle on the take-up roller for 10 min (FIG. 6C).

### Characterization of Wet Electrospun Yarns.

Morphology. The morphologies of wet electrospun bundles were analyzed by scanning electron microscopy (SEM, Phenom ProX, USA) at an accelerating voltage of 10 kV after vacuum drying (Isotemp 282A, Fisher Scientific, USA) and gold sputtering (Cressington 108, U.K.).

Thermal Property. The thermal properties of wet electrospun yarns were analyzed under differential scanning calorimetry (DSC, Chip DSC 10, Linseis, Germany) by heating the samples to 100° C. at a heating rate of 10° C./min under nitrogen flow. The degree of crystallinity was estimated by assuming that the melting enthalpy per unit mass of a pure crystalline PCL sample was identical to that of a 100% crystalline PCL sample (139.5 J/g) [49′].

Mechanical Property. The tensile properties of wet electrospun yarns were analyzed using a universal testing machine (ESM303, MARK-10, USA) at a rate of 20 mm/s until failure. The testing sample was fixed in an aperture card according to ASTM C1557 [50′]. The maximum stress and maximum strain were calculated from the maximum stress value before failure and the corresponding strain value.

Degradation Behavior. The degradation rate of wet electrospun yarns was analyzed by weighing the weight loss of samples in a Trypsin-EDTA solution. The initial weight of each sample was weighed. Then, the samples were submerged in Trypsin-EDTA and placed in an incubator at 37° C. Every week, the samples were brought out from Trypsin-EDTA and rinsed with distilled water. The samples were weighed after vacuum drying.

Cell Cultivation on Wet Electrospun Yarns. To test the biological properties of wet electrospun yarns, the viability, elongation, and alignment of cells cultured on wet electrospun yarns were evaluated. ECs were used for the experiment. ECs were cultured in the endothelial cell growth medium and expanded under standard culture conditions in a humidified atmosphere with 5% CO₂ at 37° C.

Before seeding ECs on wet electrospun yarns, the method cut yarns into 10 mm and placed them in a 24-well plate. Then, the yarns were sterilized by exposing them to ultraviolet (UV) light for 30 min. Passage 15 ECs were harvested by trypsin-EDTA and suspended in culture media at a density of 5×10⁶ cells/mL. 10 μL of the cell suspension was pipetted to each well of the 24-well plate. Four hours after the cells were attached to the yarns, the method added 50 μL of fresh culture media to each well and incubated the cell-laden yarns. The media were changed every day. Cells cultured on Petri dishes were used as a control group. Cell viability was analyzed using the LIVE/DEAD Viability/Cytotoxicity Kit. Cell elongation and alignment were analyzed using Alexa Fluor 488 phalloidin and DAPI. The cellular elongation was determined by measuring the ratio of the longest line to the shortest line across the cell nucleus. The cellular alignment was determined by measuring the angle between the long axis of the cell and the direction of aligned fibers. After staining the cells, the sample was observed using a fluorescence microscope (BX53, Olympus, Japan). Quantitative analysis was performed using ImageJ software (NIH, USA).

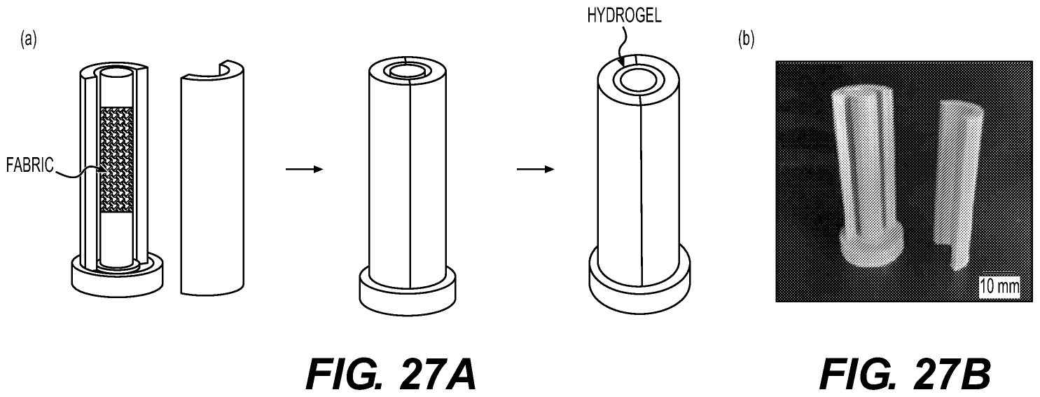

Vascular Scaffold Fabrication. The fabricated wet electrospun yarns were knitted into a circular fabric using a custom spoon loom and then placed the circular fabric onto a cylindrical part of a mold (FIGS. 27A-27B). The outer shells of the mold were assembled and injected with a hydrogel solution into the mold. After the hydrogel solidified, the tubular construct was released from the mold. In this experiment, the hydrogel solution was prepared by mixing a gelation solution (10%, w/w in deionized water) and a TG solution (6%, w/w in deionized water) at a weight ratio of 1:1. The hydrogel solution was solidified in a refrigerator at −80° C. for 20 min.

Characterization of Vascular Scaffolds. Mechanical Property. The tensile properties of scaffolds under hydrated conditions were analyzed at a rate of 20 mm/s until failure. Both the longitudinal and circumferential directions were tested.

Cell Cultivation on Scaffolds. To test the biological performance of vascular scaffolds, ECs were seeded in the lumen of the scaffolds and cultured cell-laden scaffolds. Before seeding cells, vascular scaffolds were placed in Petri dishes, and the result was sterilized by being exposed UV light for 2 h. Passage 15 ECs were harvested by trypsin-EDTA and suspended in culture medium at a density of 5×10⁶ cells/mL. 3 mL of the cell suspension was pipetted to the lumen of each vascular scaffold. The method rotated vascular scaffolds by 90° every hour. After 4 h, the method added 10 mL of fresh media to each Petri dish and incubated the cell-laden scaffolds. The media were changed every day. Cell proliferation was analyzed using CellTracker CM-Dil Dye.

Statistical Analysis. All experiments were performed with five replicates for each sample. Statistical comparisons were performed using Student's t-test. The threshold of p values for statistical significance was set at 0.05.

### Results and Discussion

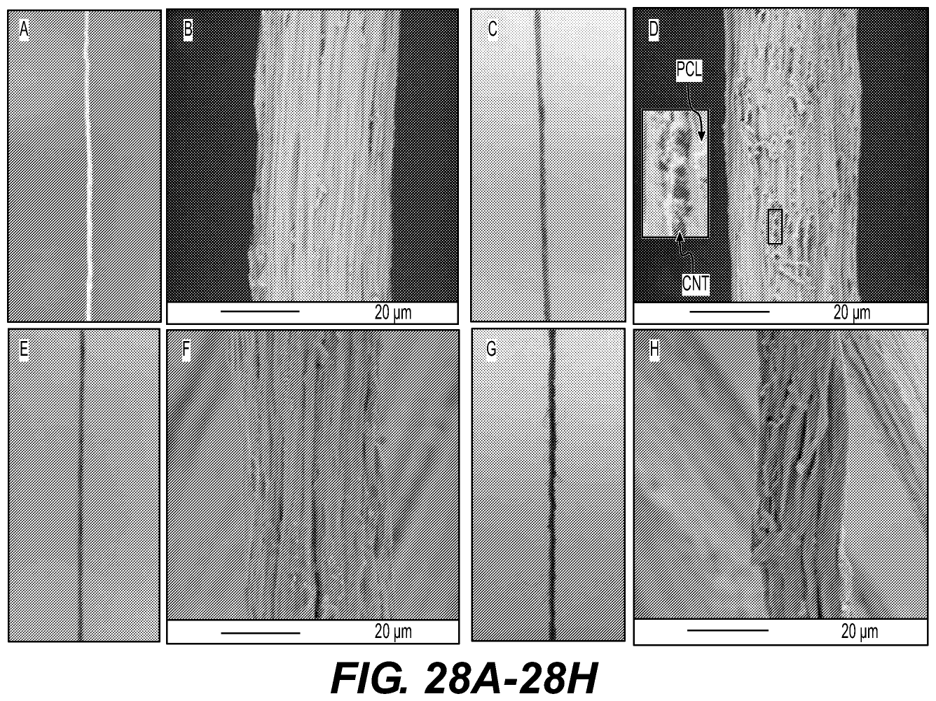

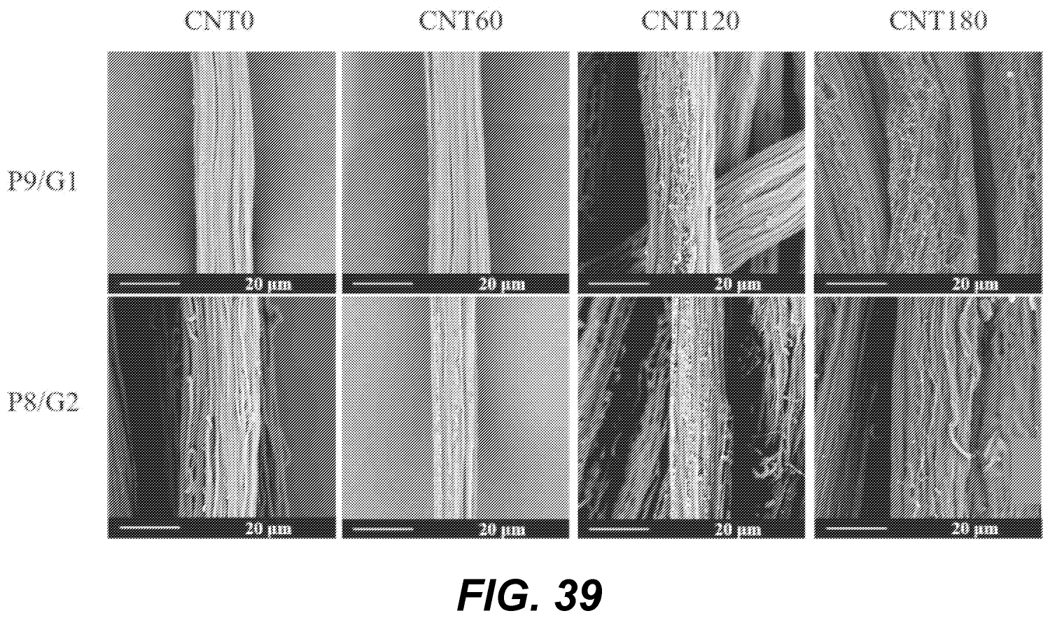

Morphologies and Thermal Properties of Wet Electrospun Yarns. The study observed a noticeable change in the color of wet electrospun yarns from white to black (FIGS. 28A-28H and 39) when the concentrations of CNTs were increased in the wet electrospinning bath. SEM images showed the appearance of scattered black CNT spots on the bundle surface at a CNT concentration of 60 mg/L. CNTs covered most bundle surfaces at a concentration of 120 mg/L and covered the entire surface at 180 mg/L.

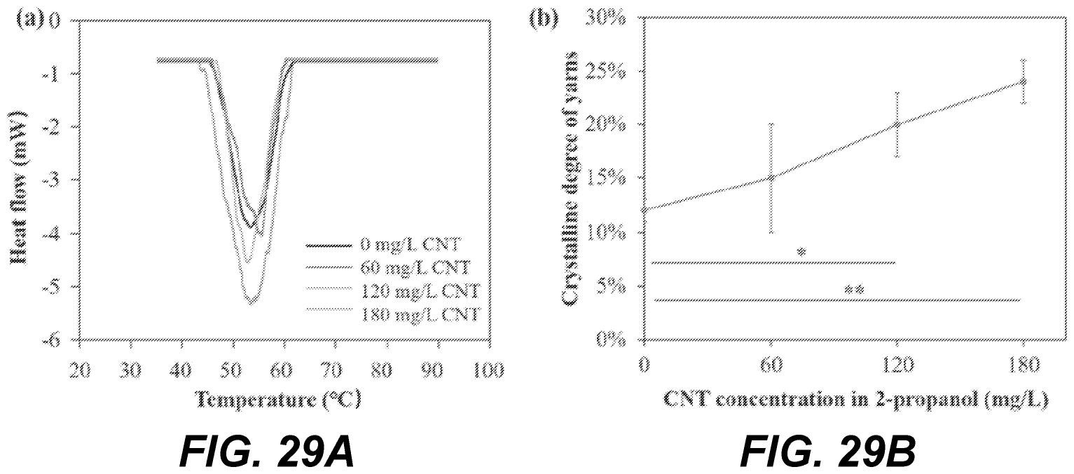

To investigate whether the presence of CNTs on wet electrospun yarns affected the thermal properties of yarns, DSC tests were performed of PCL yarns fabricated from baths with different CNT concentrations (FIG. 29A). The results (FIG. 29B) showed that the crystalline degrees of wet electrospun PCL yarns fabricated from CNT120 and CNT180 baths were significantly larger than those of yarns in the control group without CNTs). However, no significant difference in the crystalline degree existed between yarns fabricated from the CNT60 bath and the control group. The results demonstrate that CNTs in the bath can significantly affect the thermal properties of wet electrospun yarns when the CNT concentration is above a certain threshold.

Fabrication methods such as coaxial spinneret printing [51′] and in situ electrospinning [52′] have successfully encapsulated a CNT core in a polymeric shell, but no interactions between CNTs and the polymer occurred. In wet electrospinning, CNT-induced polymer crystallization occurred on the fiber surface. When PCL fibers were electrospun into the CNT bath, CNTs absorbed on the fiber surface altered the crystallization kinetics as well as the resultant crystal structure and crystalline degree of PCL yarns [53′-54′]. Other research groups achieved CNT-induced polymer crystallization by dispersing CNTs into polymer matrixes [55′-57′]. In our study, CNTs were introduced during the solidification process of polymer fibers instead of dispersing CNTs into polymer matrixes, eliminating the requirement of using miscible solvents to dissolve CNTs and polymers.

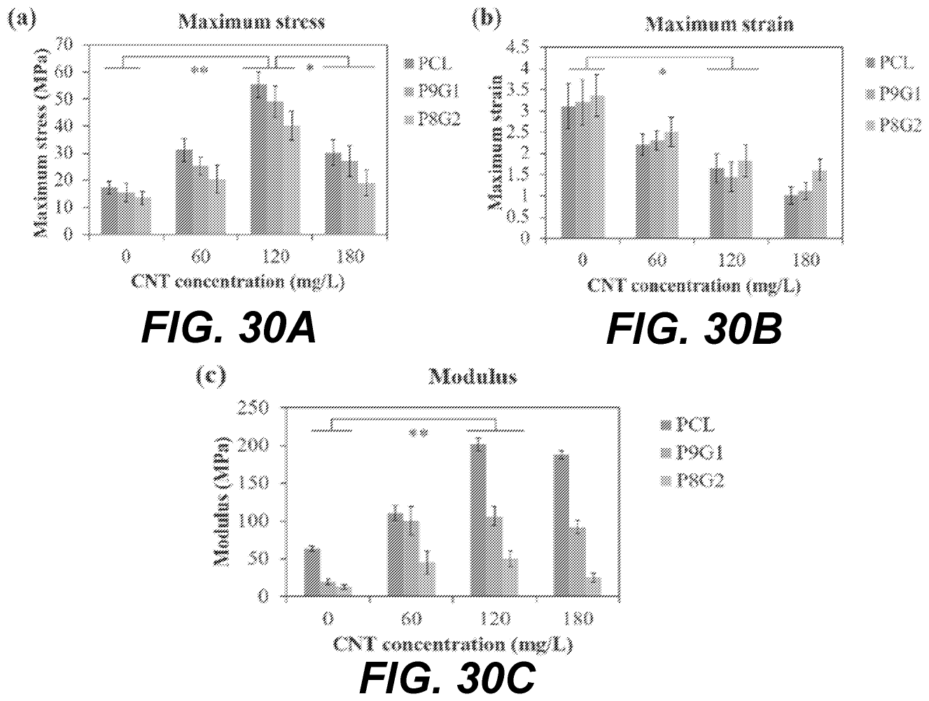

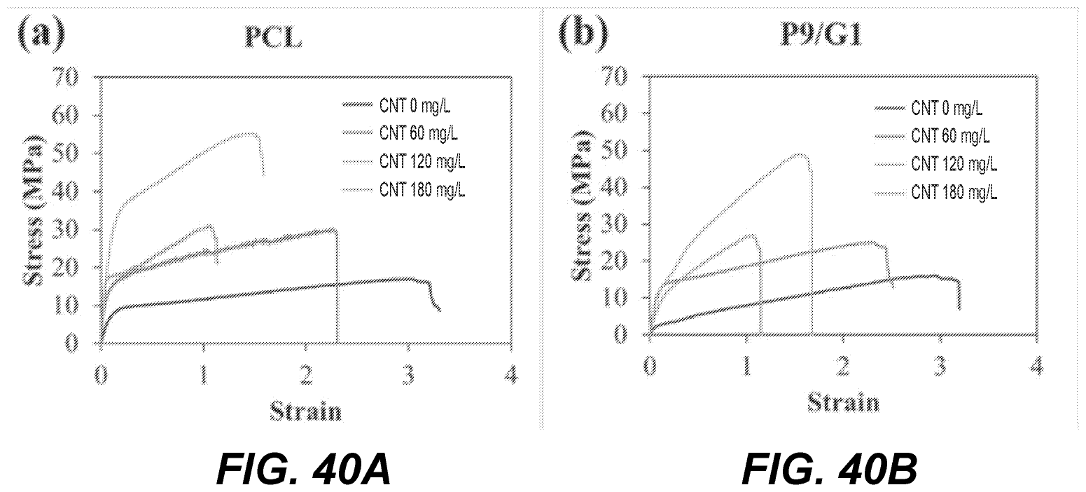

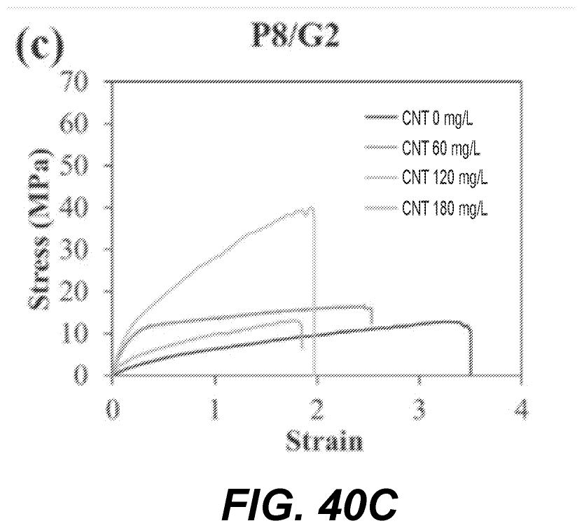

Mechanical Properties of Wet Electrospun Yarns. Tensile tests were conducted to investigate the mechanical properties of wet electrospun yarns (FIGS. 40A-40C). FIGS. 30A-30C show the maximum stress, maximum strain, and modulus of each type of wet electrospun yarns. The maximum stress and modulus of yarns decreased as the gelatin concentration increased. The negative correlation is mainly because PCL is a more elastic material with higher mechanical strength than gelatin [58′]. Thus, increasing the concentration of gelatin in PCL leads to a decreased maximum stress and modulus.

Mechanical properties of wet electrospun yarns were also affected by the CNT concentration in the bath. As the CNT concentration increased in the bath, the maximum stress and modulus of yarns first started to increase and then decreased after reaching a peak, while the maximum strain of yarns decreased to a low plateau and then remained stable. Other research groups have demonstrated that the mechanical properties of polymer/CNT composite materials were largely dependent on the content of CNTs [59′-61′]. Within a limited weight fraction of CNTs, the maximum strength and modulus of composites significantly increase with the addition of CNTs. The thread morphologies of CNTs can overlap with each other, and strong π-π bond effects and van der Waals forces among adjacent nanotubes can lead to interweaving between nanotubes, resulting in a network-like structure. The structure has an excellent stress-transfer effect, improving the tensile strength and modulus of composites [62′]. However, when the weight fraction of CNTs exceeds a certain value, cluster structures are formed, weakening the reinforcement of CNTs. The decrease of the composites' maximum strain can be explained by the fact that brittle CNTs in an elastic polymer matrix restrict the plastic deformation of the matrix [63′].

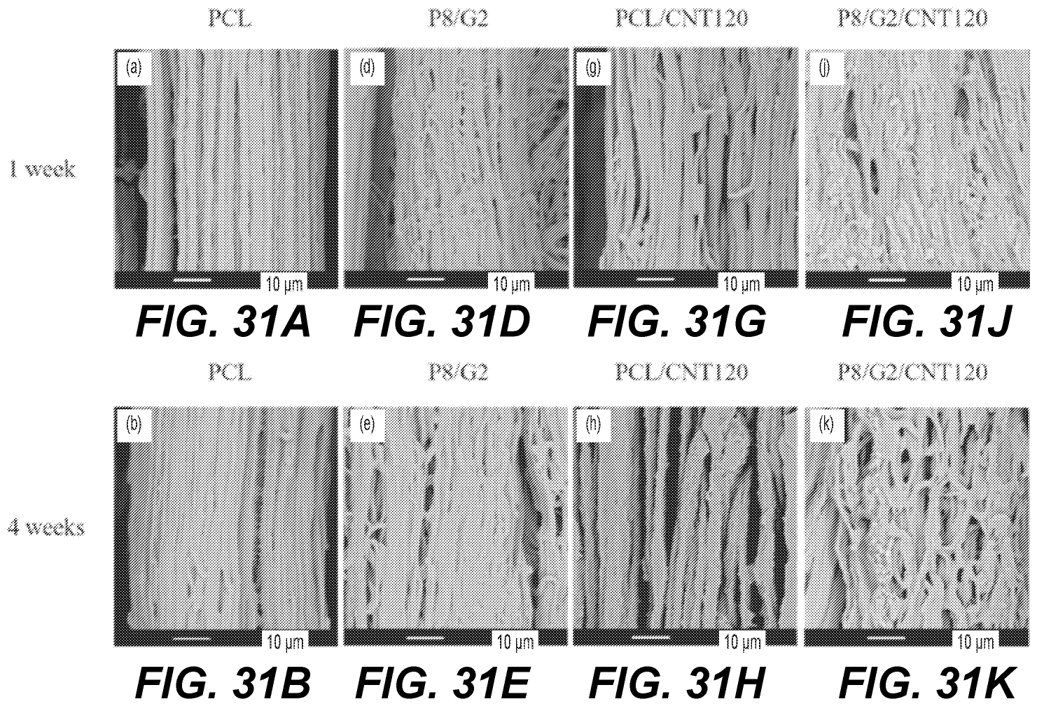

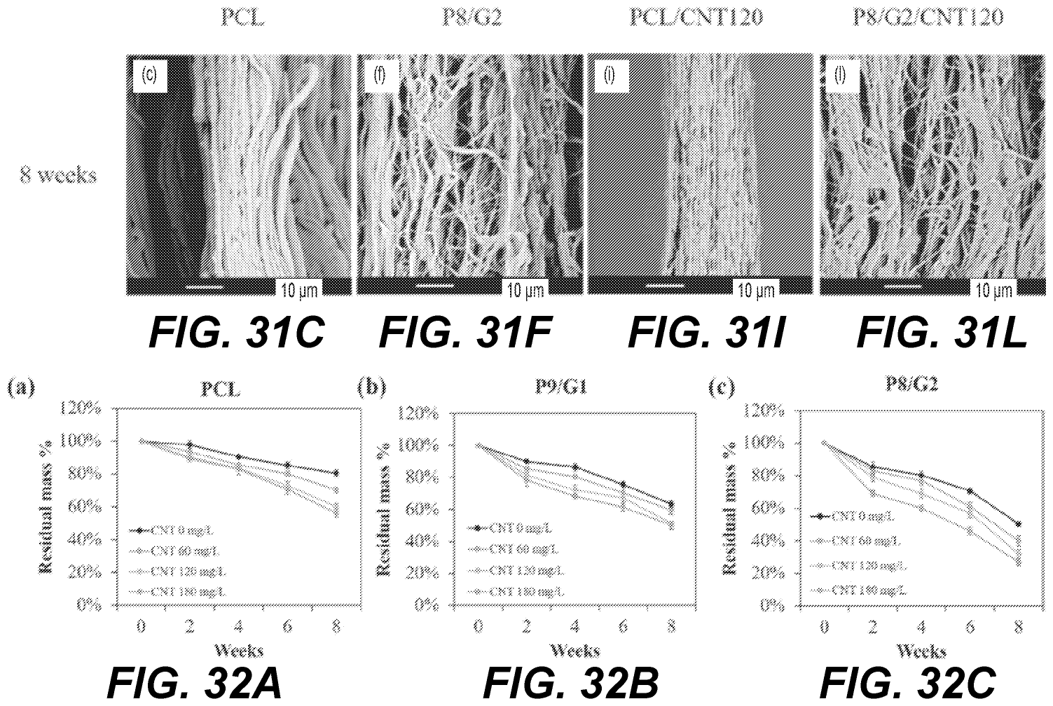

Degradation Behavior of Wet Electrospun Yarns. Scaffold degradability is an important parameter for tissue engineering as the scaffolds need to gradually degrade with tissue regeneration after implantation. The degradation behavior of each type of wet electrospun yarns was analyzed. FIGS. 31A-31L show the change in the surface morphology of wet electrospun yarns after degradation. Images of pure PCL yarns (FIGS. 31A-31C) showed that fibers became thinner after 8 weeks of degradation, suggesting a possible surface erosion of the pure PCL yarns. When gelatin was added to the pure PCL yarns (FIGS. 31D-31F), a fusion of fibers was observed after 4 weeks of degradation, and the fibrous morphology was destroyed after 8 weeks of degradation. This suggests the possibility of bulk erosion of the PCL/gelatin yarns. Images of PCL/CNT yarns (FIGS. 31G-31I) showed that the fiber surface became rougher with further degradation. When gelatin was added to the PCL/CNT yarns (FIGS. 31J-31L), both fiber fusion and surface roughening were observed after 4 weeks of degradation.

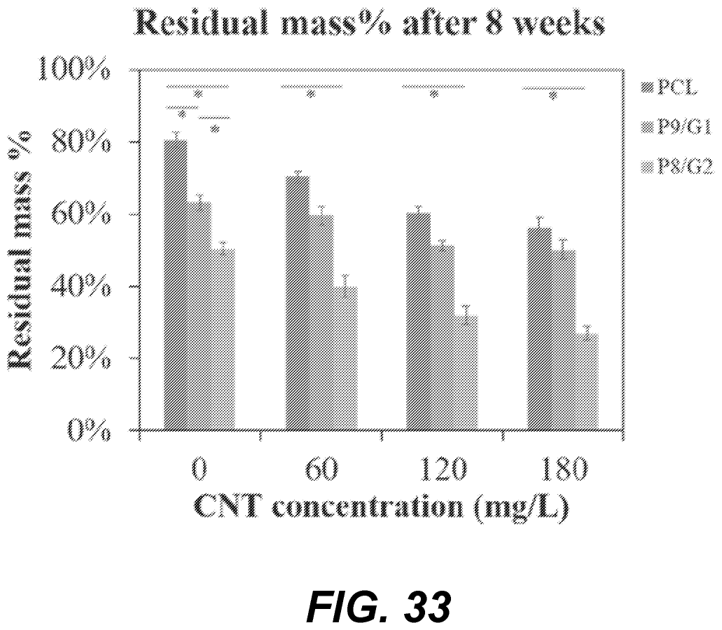

FIG. 32 shows the residual mass (%) of each type of wet electrospun yarns. The addition of CNTs accelerated the degradation of yarns, which can be attributed to potentially higher enzyme binding to the CNTs in the polymer/CNT composite yarns [56′]. FIG. 33 shows the residual mass (%) of wet electrospun yarns after 8 weeks of degradation. The addition of gelatin accelerated the degradation of yarns because gelatin increased the hydrophilicity of PCL yarns. Nearly 73% of weight loss occurred after 8 weeks of degradation, indicating a rapid rate of degradation of P8/G2/CNT180.

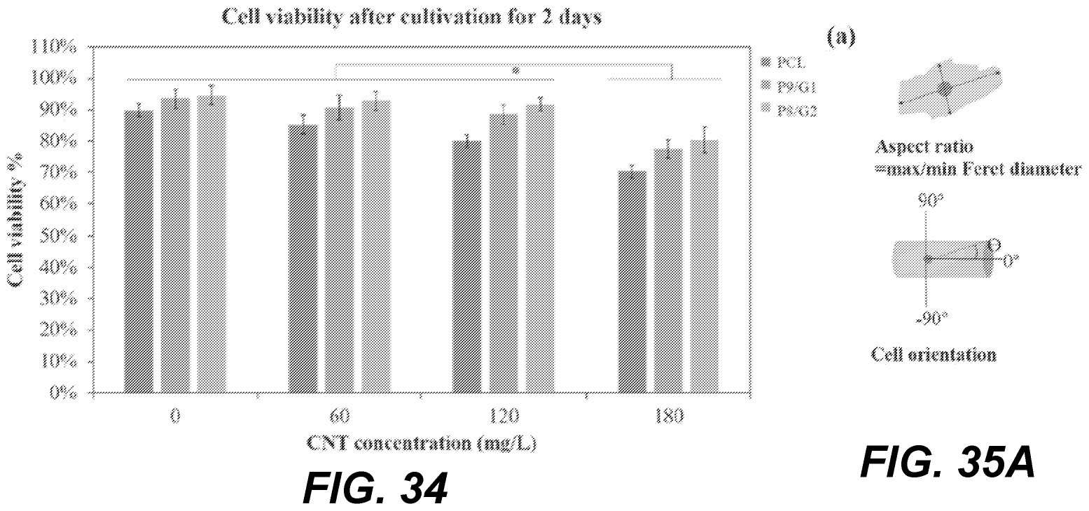

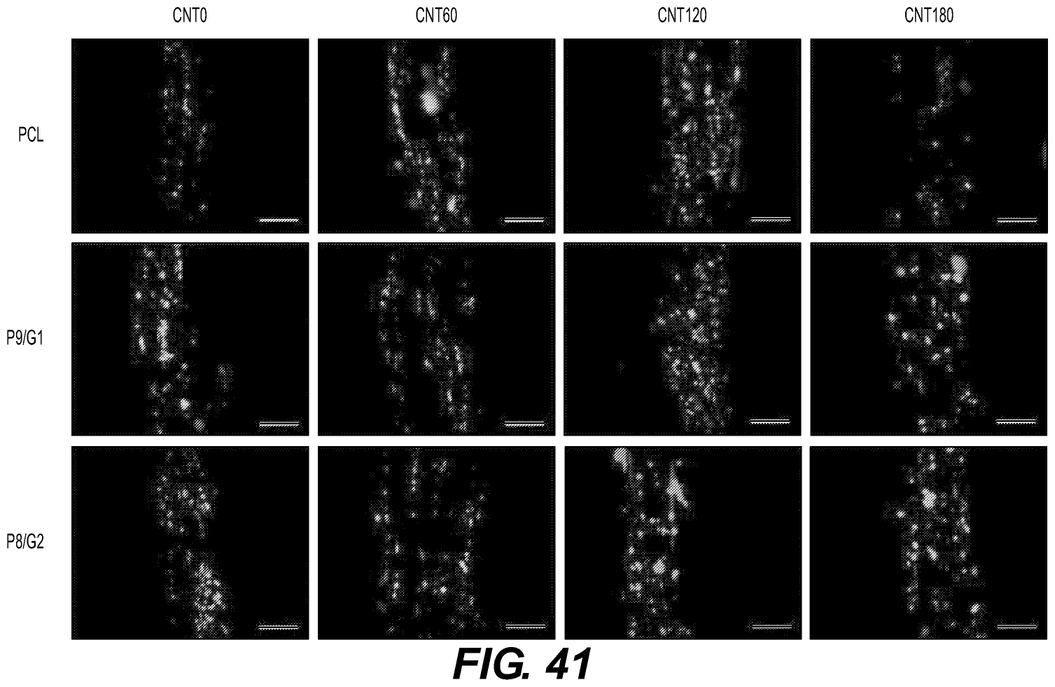

Cell Viability, Elongation, and Alignment on Wet Electrospun Yarns. To investigate the effect of the composition of wet electrospun yarns on cell viability, elongation, and alignment, ECs were seeded on wet electrospun yarns with different compositions. After culturing for 2 days, the live/dead fluorescence assay was performed to measure the cell viability (FIGS. 41 and 34). The results indicated that within a limited weight fraction of CNTs, CNTs have no significant impact on cell viability. However, cell viability significantly decreased when the concentration of CNTs reached up to 180 mg/L in the bath. A possible explanation is that the high concentration of CNTs in the bath leads to an agglomeration of CNTs on the surface of wet electrospun yarns, creating a toxic surrounding environment for the cells. Such concentration-dependent cytotoxicity of CNTs has also been demonstrated in other studies,^{64′-66′} indicating a critical CNT concentration specific to each cell type.

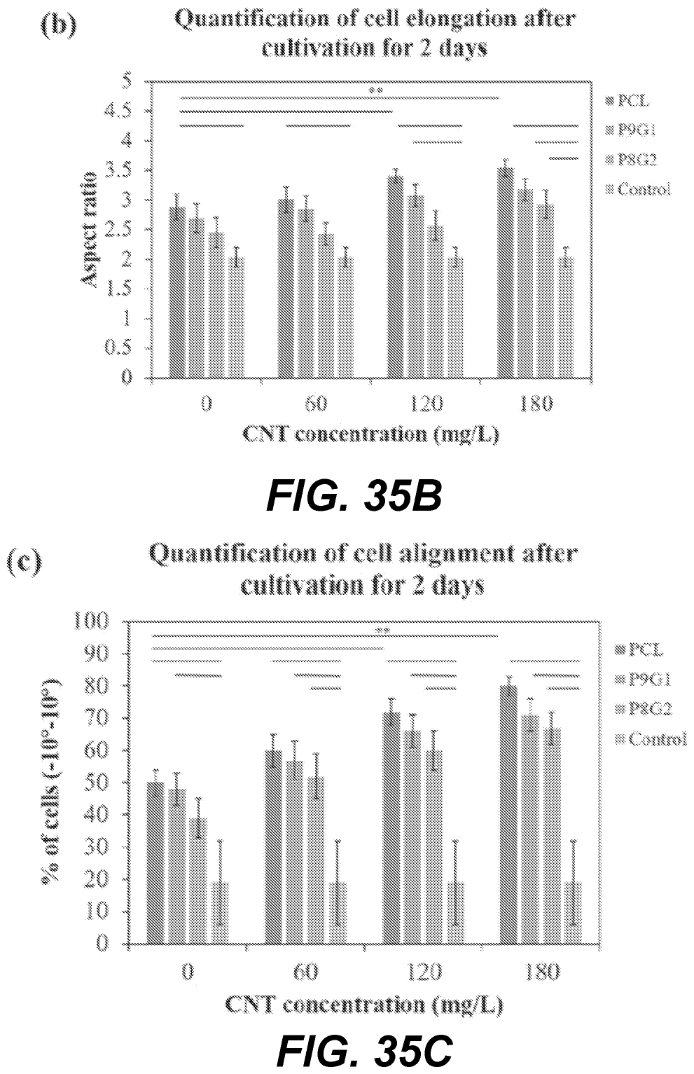

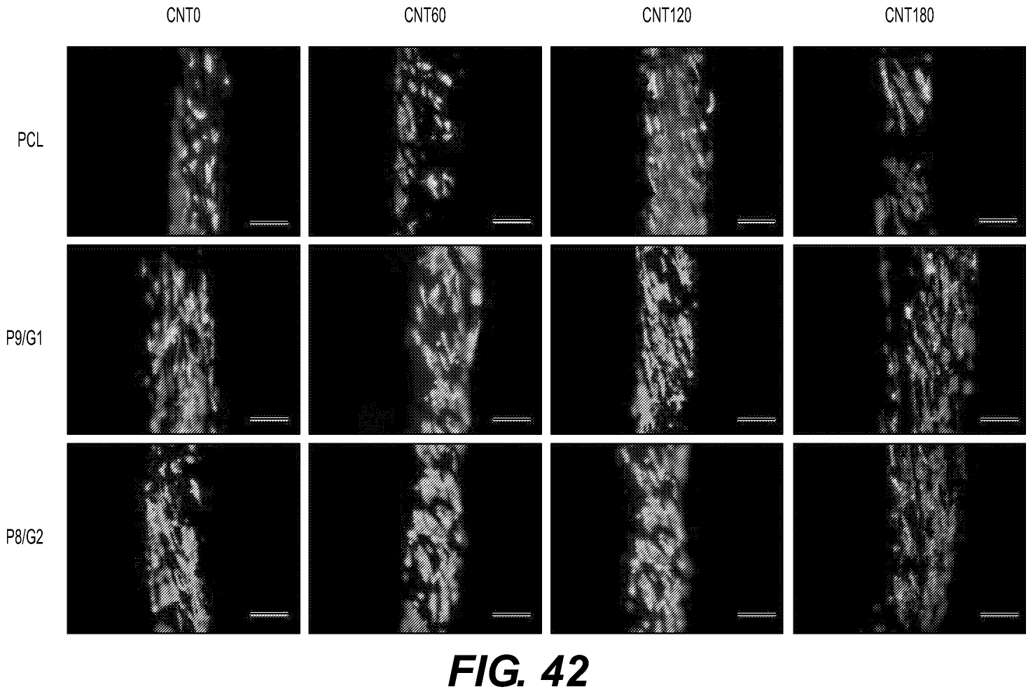

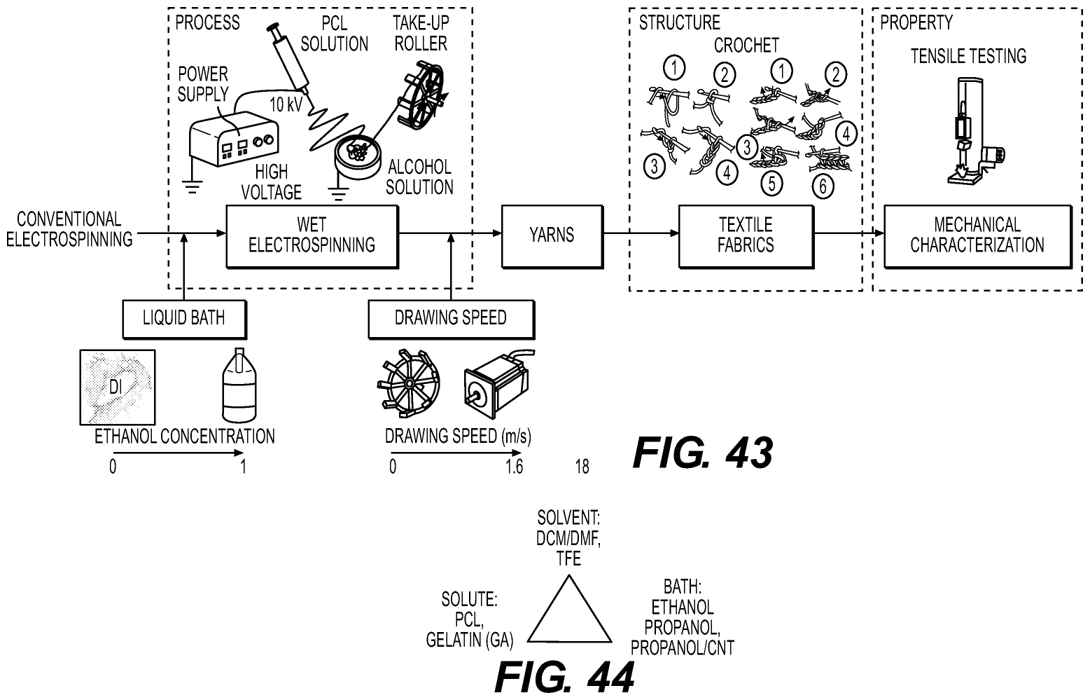

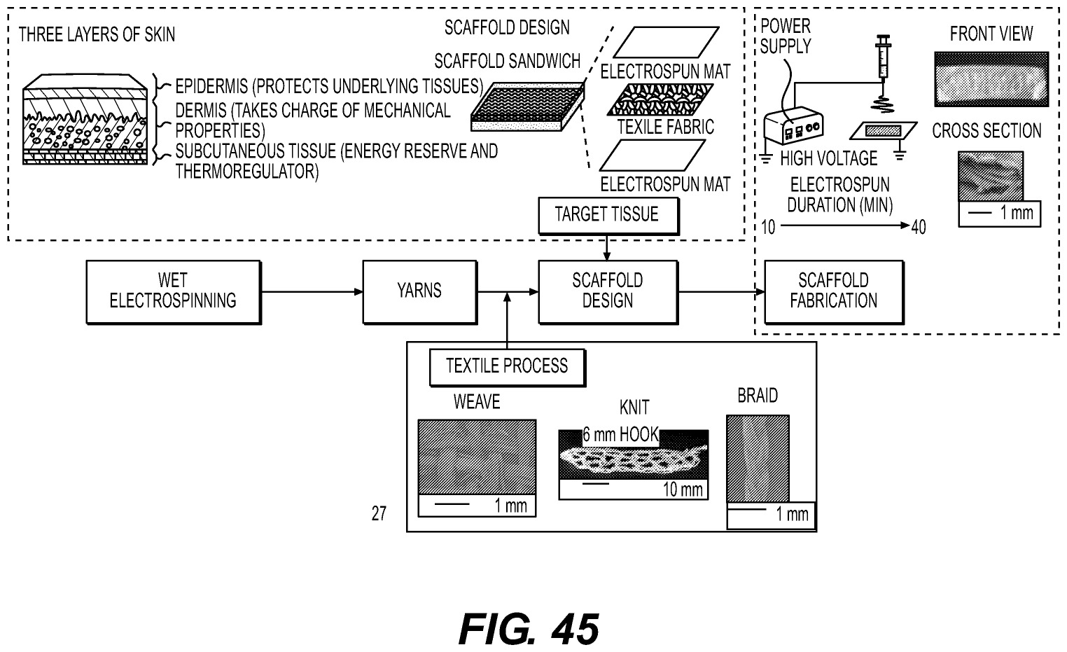

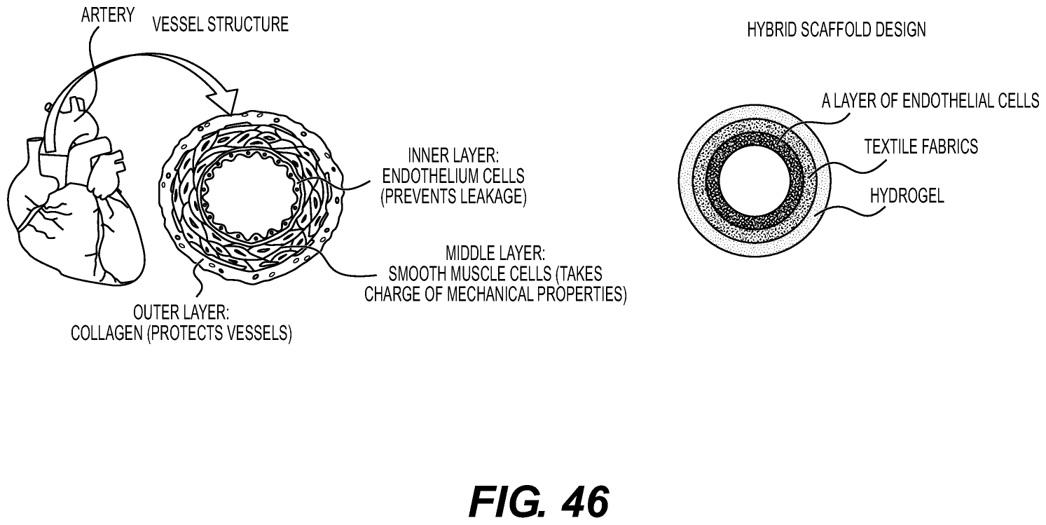

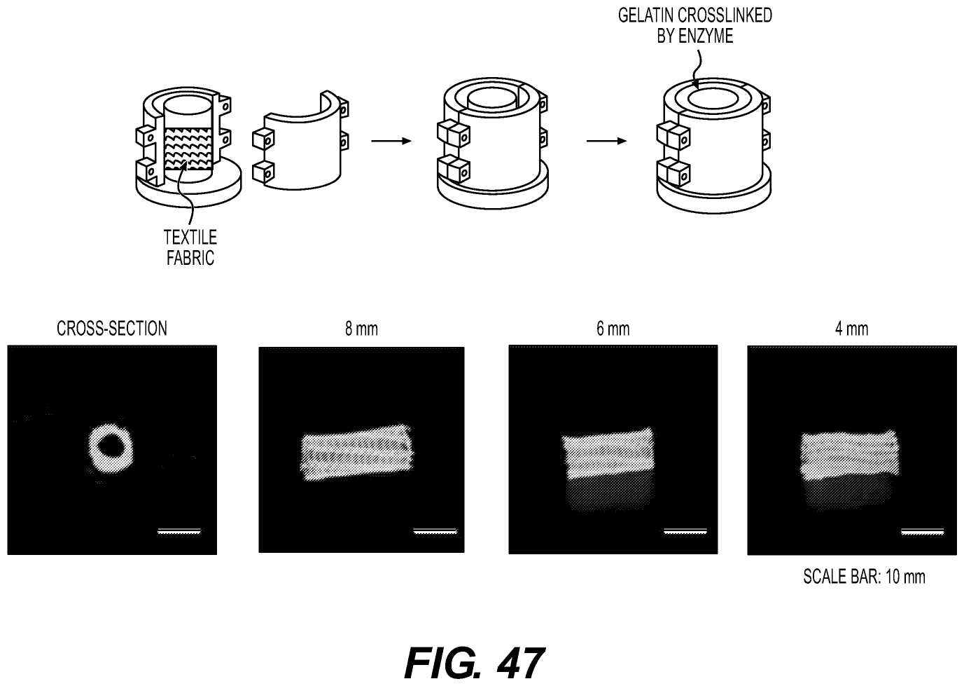

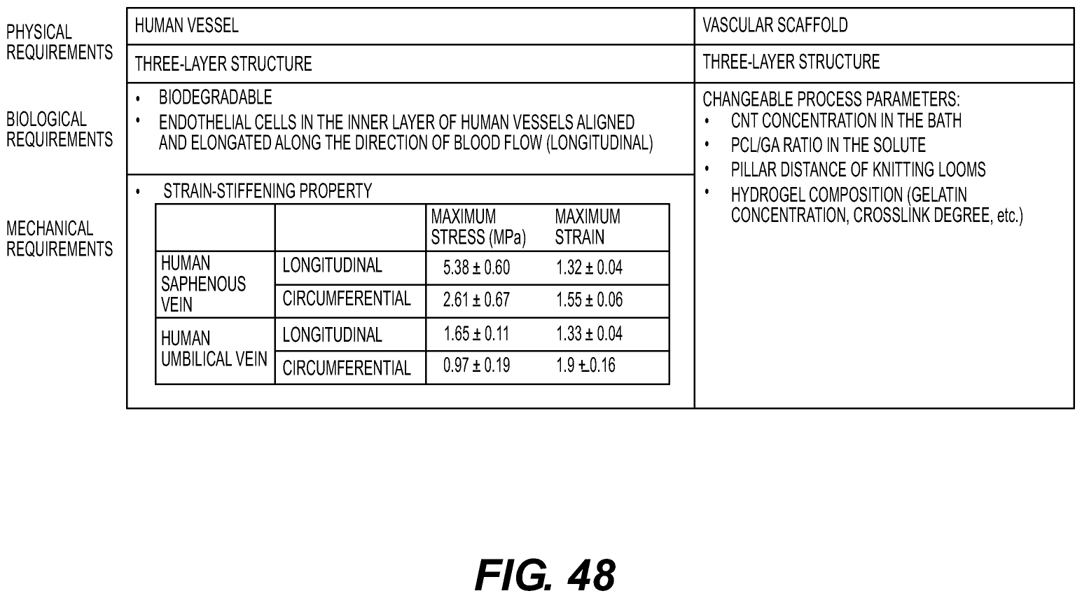

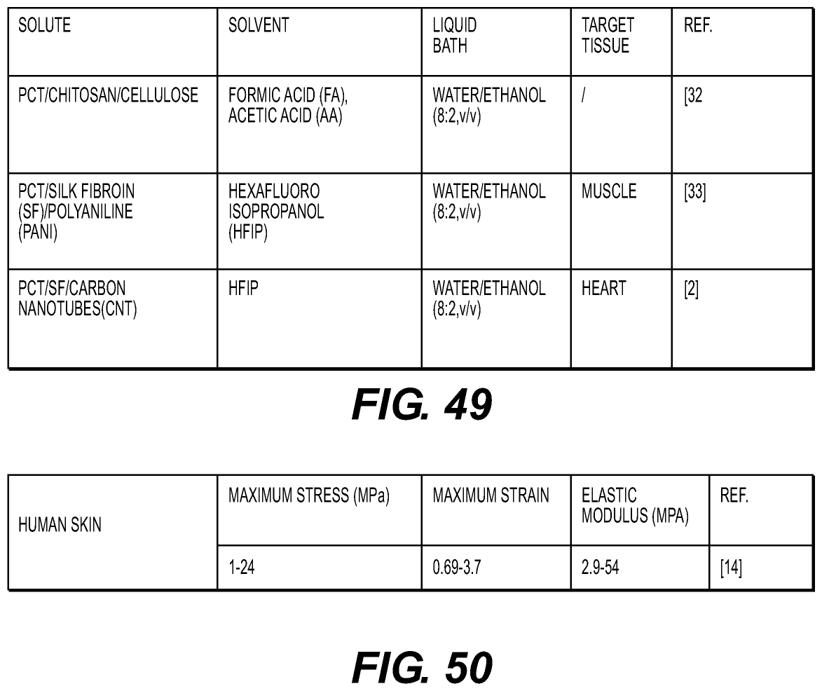

After culturing for 2 days, Alexa Fluor and DAPI assays were performed to measure the cell elongation and alignment (FIG. 42). Quantitative analysis of cell elongation on wet electrospun yarns was performed by the cellular aspect ratio, which is defined as the ratio between the length of the longest line and the shortest line across the nuclei (FIG. 35A). Cells grown on Petri dishes were used as a control group. The results (FIG. 35B) showed that cells grown on PCL yarns had significantly higher aspect ratios than those on Petri dishes. The aspect ratios of cells grown on PCL yarns increased from 2.89±0.21 to 3.54±0.14 when the CNT concentration in the bath increased from 0 to 180 mg/L. It was also found that cellular aspect ratios decreased with the addition of gelatin, possibly due to the fusion of PCL/gelatin fibers in culture media.

Quantitative analysis of cell alignment on wet electrospun yarns was estimated by the orientation of cells by measuring the angle between the long axis of cells and the direction of aligned fibers (FIG. 35A). Cells that angled within ±100 were considered to be aligned. The results (FIG. 35C) showed that cell alignments on PCL yarns were significantly higher than those on Petri dishes. When no CNT existed in the bath, roughly 50% of cells aligned within a ±10° orientation. When the CNT concentrations increased to 120 and 180 mg/L, roughly 72 and 80% of cells aligned within a ±10° orientation. It was also found that cell alignments on PCL yarns decreased with the addition of gelatin.

Controlled cell organization plays a critical role in the microarchitecture of many human tissues such as vessels [67′] and connective tissues [68′], deciding their mechanical and biological function [69′]. Many critical components of the cell organization, such as cell elongation and cell alignment, can be modulated with the aid of a patterned substrate [70′], flow rate [71′], electrical stimulation [72′], and cyclic stress [73′]. Other research groups have shown that wet electrospun yarns supported cell elongation and alignment because of their anisotropic fibrous structures [42′, 74′]. In the current study, the inclusion of CNTs within wet electrospun yarns was found to enhance the elongation and alignment of ECs, suggesting that CNTs exerted more facilitative cues for directing cells into anisotropic arrangements. Future work should focus on whether topographical cues or the conductivity of CNTs accounts for the enhanced cell elongation and alignment.

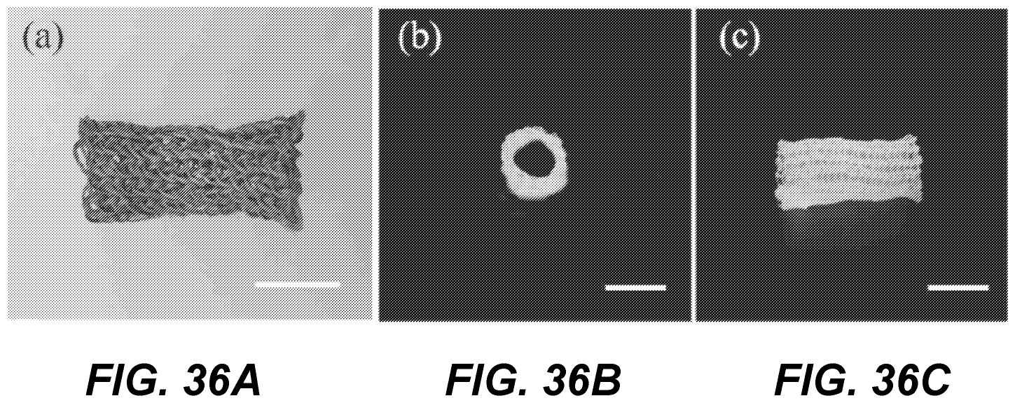

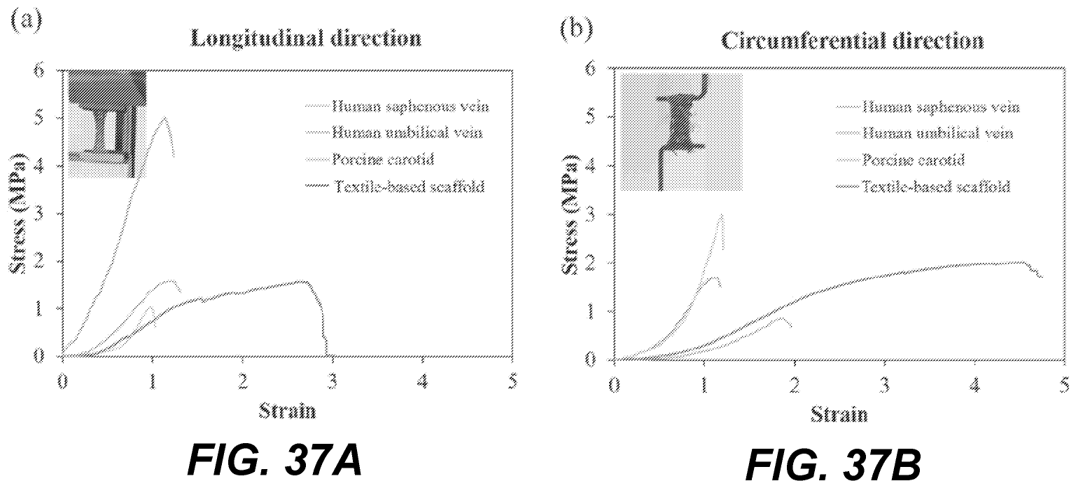

Mechanical Properties of Scaffolds. The study used P8/G2/CNT120 yarns to fabricate the textile fabric for the vascular scaffold (FIGS. 36A-36C). The study measured the stress-strain curves for the textile-based scaffolds and compared them with those of native vessels (FIGS. 37A-37B). The textile-based scaffold displayed the characteristic J-shape curve of native vessels. A comparison of maximum stress and strain between various native vessels and the scaffold is displayed in Table 9. The maximum stress of the scaffold in the longitudinal direction was significantly lower than that of the human saphenous vein but comparable to that of the human umbilical vein [75′]. The maximum strains of the scaffold in both longitudinal and circumferential directions were significantly higher than those of native vessels [75′, 76′]. Adding stiffer materials, such as collagen, to the yarn materials or the hydrogel part of the vascular scaffold can potentially improve the maximum stress of the scaffold.

TABLE 9
Comparison of the Maximum Stress and Strain of Various
Native Vessels and Textile-Based Scaffolds^{a}

| | | Maximum Stress | Maximum |
|---|---|---|---|
| | | (MPA) | Strain |
| Human saphenous vein [75′] | Longitudinal | 5.38 ± 0.60** | 1.32 ± 0.04** |
| | Circumferential | 2.61 ± 0.67 | 1.55 ± 0.06** |
| Human umbilical vein [75′] | Longitudinal | 1.65 ± 0.11 | 1.33 ± 0.04** |
| | Circumferential | 0.97 ± 0.19 | 1.9 ± 0.16** |
| Porcine carotid [76′] | Longitudinal | 0.95 ± 0.13 | 1.05 ± 0.11** |
| | Circumferential | 2.59 ± 0.31 | 1.25 ± 0.15** |
| Textile-based scaffold | Longitudinal | 1.56 ± 0.43 | 2.66 ± 0.33 |
| | Circumferential | 1.61 ± 0.59 | 4.55 ± 0.15 |

^{a}Compared to the scaffolds in the same orientation,
**represents p < 0.01.

Under tensile stress, native vessels will generally display the nonlinear or J-shape tensile curve to protect vessels from mechanical trauma [77′-80′]. In this study, the fabricated scaffold showed a similar nonlinear mechanical behavior to the native vessels because the initial stress was used to align the curved yarns in the textile fabric. Many research groups have used textile-based scaffolds to recapitulate human tissues' nonlinear mechanical properties [47′, 48′, 81′-83′]. However, most of them used commercial yarns with a limited range of material properties that were hard to customize. In this study, the wet electrospun yarns was fabricated with sufficient mechanical strength to withstand the textile fabrication process. In addition, the study evaluated the tuning of the mechanical properties of yarns by changing the content and ratio of materials in the wet electrospinning solute and bath. To the inventor's knowledge, this is the first study to add functional materials in the bath of wet electrospinning to improve the properties of yarns, providing a novel method to customize yarns for constructing textile-based scaffolds.

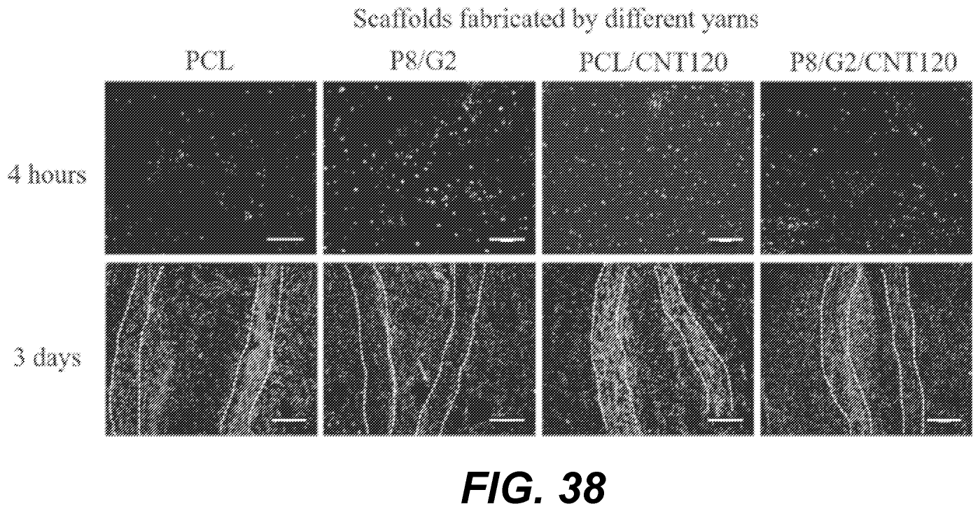

Cell Proliferation on Scaffolds. To investigate cell proliferation on the scaffolds, ECs were seeded in the lumen of the scaffolds fabricated by different yarns. CellTracker CM-Dil Dye was used to stain both cells on scaffolds after culturing for 4 h and cells after culturing 3 days (FIG. 38). Images taken at 4 h of culturing showed cells attached well and scattered randomly on the scaffolds, indicating the scaffold's good biocompatibility. Images taken at 3 days of culturing showed cells spread well on the scaffolds and aligned with the yarn directions on the textile fabrics. These observations corroborated the findings that the yarns supported cell alignment.

The coronary artery wall consists of three layers [84′]. The outer layer is composed of elastic and collagen fibers, which can protect the artery. The middle layer comprises smooth muscle and elastic fibers, responsible for maintaining the artery's mechanical properties. The inner layer is composed of a membrane of aligned endothelial cells [85′], which can protect the artery from inflammation and permeability [86′]. The fabricated textile-based scaffold resembles the structure of the coronary artery and displays similar properties. The hydrogel layer is responsible for protecting the scaffold, while the textile fabric maintains its mechanical strength. The biocompatibility of the hydrogel and textile fabric supports cell attachment and proliferation, making it possible to form a confluent cell membrane in the lumen of the scaffold. Furthermore, the inclusion of CNTs in the textile fabrics improves cell alignment to a closer match with the cell arrangement of the human arteries. Overall, the textile-based scaffold with a similar structure and similar properties to the human arteries can be a good candidate for vascular tissue engineering.

### REFERENCES

[1′] CDC. Underlying Cause of Death 2000-2013 on CDC WONDER Online Database. http://www.cdc.gov/nchs/data/health_policy/AADR_drug_poisoning_involving_OA_Heroin US_2000-2014.pdf (accessed Sep. 5, 2020).
[2′] Virani, S. S.; Alonso, A.; Benjamin, E. J.; Bittencourt, M. S.; Callaway, C. W.; Carson, A. P.; Chamberlain, A. M.; Chang, A. R.; Cheng, S.; Delling, F. N.; Djousse, L.; Elkind, M. S. V.; Ferguson, J. F.; Fornage, M.; Khan, S. S.; Kissela, B. M.; Knutson, K. L.; Kwan, T. W.; Lackland, D. T.; Lewis, T. T.; Lichtman, J. H.; Longenecker, C. T.; Loop, M. S.; Lutsey, P. L.; Martin, S. S.; Matsushita, K.; Moran, A. E.; Mussolino, M. E.; Perak, A. M.; Rosamond, W. D.; Roth, G. A.; Sampson, U. K. A.; Satou, G. M.; Schroeder, E. B.; Shah, S. H.; Shay, C. M.; Spartano, N. L.; Stokes, A.; Tirschwell, D. L.; VanWagner, L. B.; Tsao, C. W.; Wong, S. S.; Heard, D. G. Heart Disease and Stroke Statistics 2020 Update. A Report from the American Heart Association. Circulation 2020, 141, E139-E596.
[3′] Dozois, M. D.; Bahlmann, L. C.; Zilberman, Y; Tang, X.; Shirley. Carbon Nanomaterial-Enhanced Scaffolds for the Creation of Cardiac Tissue Constructs: A New Frontier in Cardiac Tissue Engineering. Carbon 2017, 120, 338-349.
[4′] Torgbo, S.; Sukyai, P. Bacterial Cellulose-Based Scaffold Materials for Bone Tissue Engineering. Appl. Mater. Today 2018, 11, 34-49.
[5′] Asghari, F.; Samiei, M.; Adibkia, K.; Akbarzadeh, A.; Davaran, S. Biodegradable and Biocompatible Polymers for Tissue Engineering Application: A Review. Artif. Cells, Nanomed., Biotechnol. 2017, 45, 185-192.
[6′] Balint, R.; Cassidy, N. J.; Cartmell, S. H. Conductive Polymers: Towards a Smart Biomaterial for Tissue Engineering. Acta Biomater. 2014, 10, 2341-2353.
[7′] Martins, A. M.; Eng, G.; Caridade, S. G.; Mano, J. F.; Reis, R. L.; Vunjak-Novakovic, G. Electrically Conductive Chitosan/Carbon Scaffolds for Cardiac Tissue Engineering. Biomacromolecules 2014, 15, 635-643.
[8′] Kai, D.; Prabhakaran, M. P.; Jin, G.; Ramakrishna, S. Polypyrrole-Contained Electrospun Conductive Nanofibrous Mem-branes for Cardiac Tissue Engineering. J. Biomed. Mater. Res., Part A 2011, 99, 376-385.
[9′] Qazi, T. H.; Rai, R.; Dippold, D.; Roether, J. E.; Schubert, D. W.; Rosellini, E.; Barbani, N.; Boccaccini, A. R. Development and Characterization of Novel Electrically Conductive PANI-PGS Composites for Cardiac Tissue Engineering Applications. Acta Biomater. 2014, 10, 2434-2445.
[10′] Zhao, Y; Wang, S.; Guo, Q.; Shen, M.; Shi, X. Hemocompatibility of Electrospun Halloysite Nanotube- and Carbon Nanotube-Doped Composite Poly(Lactic-Co-Glycolic Acid) Nano-fibers. J. Appl. Polym. Sci. 2013, 127, 4825-4832.
[11′] Zhang, Y; Yu, Y; Dolati, F.; Ozbolat, I. T. Effect of Multiwall Carbon Nanotube Reinforcement on Coaxially Extruded Cellular Vascular Conduits. Mater. Sci. Eng. C 2014, 39, 126-133.
[12′] Lu, X.; Zhu, Y; Bai, R.; Wu, Z.; Qian, W.; Yang, L.; Cai, R.; Yan, H.; Li, T.; Pandey, V.; Liu, Y; Lobie, P. E.; Chen, C.; Zhu, T. Long-Term Pulmonary Exposure to Multi-Walled Carbon Nanotubes Promotes Breast Cancer Metastatic Cascades. Nat. Nanotechnol. 2019, 14, 719-727.
[13′] He, S.; Hu, Y; Wan, J.; Gao, Q.; Wang, Y; Xie, S.; Qiu, L.; Wang, C.; Zheng, G.; Wang, B.; Peng, H. Biocompatible Carbon Nanotube Fibers for Implantable Supercapacitors. Carbon N. Y 2017, 122, 162-167.
[14′] Foldvari, M.; Bagonluri, M. Carbon Nanotubes as Functional Excipients for Nanomedicines: II. Drug Delivery and Biocompatibility Issues. Nanomed. Nanotechnol. Biol. Med. 2008, 4, 183-200.
[15′] Smart, S. K.; Cassady, A. I.; Lu, G. Q.; Martin, D. J. The Biocompatibility of Carbon Nanotubes. Carbon 2006, 44, 1034-1047.
[16′] Omrani, M. M.; Ansari, M.; Kordestani, S. S.; Kiaie, N.; Salati, A. Enhanced Bone Marrow Stem Cell Attachment and Differentiation on PCL/CNT Substrate. Inorg. Nano-Met. Chem. 2019, 49, 136-142.
[17′] Chao, T.-I.; Xiang, S.; Chen, C.-S.; Chin, W.-C.; Nelson, A. J.; Wang, C.; Lu, J. Carbon Nanotubes Promote Neuron Differentiation from Human Embryonic Stem Cells. Biochem. Biophys. Res. Commun. 2009, 384, 426-430.
[18′] Chao, T.-I.; Xiang, S.; Lipstate, J. F.; Wang, C.; Lu, J. Poly(Methacrylic Acid)-Grafted Carbon Nanotube Scaffolds Enhance Differentiation of HESCs into Neuronal Cells. Adv. Mater. 2010, 22, 3542-3547.
[19′] Liang, X.; Li, H.; Dou, J.; Wang, Q.; He, W.; Wang, C.; Li, D.; Lin, J. M.; Zhang, Y Stable and Biocompatible Carbon Nanotube Ink Mediated by Silk Protein for Printed Electronics. Adv. Mater. 2020, 32, 2000165.
[20′] Shin, S. R.; Jung, S. M.; Zalabany, M.; Kim, K.; Zorlutuna, P.; Kim, S. B.; Nikkhah, M.; Khabiry, M.; Azize, M.; Kong, J.; Wan, K.-t.; Palacios, T.; Dokmeci, M. R.; Bae, H.; Tang, X.; Khademhosseini, A. Carbon-Nanotube-Embedded Hydrogel Sheets for Engineering Cardiac Constructs and Bioactuators. ACS Nano 2013, 7, 2369-2380.
[21′] Kharaziha, M.; Shin, S. R.; Nikkhah, M.; Topkaya, S. N.; Masoumi, N.; Annabi, N.; Dokmeci, M. R.; Khademhosseini, A. Tough and Flexible CNT-Polymeric Hybrid Scaffolds for Engineering Cardiac Constructs. Biomaterials 2014, 35, 7346-7354.
[22′] Sahoo, N. G.; Rana, S.; Cho, J. W.; Li, L.; Chan, S. H. Polymer Nanocomposites Based on Functionalized Carbon Nanotubes. Prog. Polym. Sci. 2010, 35, 837-867.
[23′] Gnanasekaran, K.; Heijmans, T.; van Bennekom, S.; Woldhuis, H.; Wijnia, S.; de With, G.; Friedrich, H. 3D Printing of CNT- and Graphene-Based Conductive Polymer Nanocomposites by Fused Deposition Modeling. Appl. Mater. Today 2017, 9, 21-28.
[24′] Mombini, S.; Mohammadnejad, J.; Bakhshandeh, B.; Narmani, A.; Nourmohammadi, J.; Vahdat, S.; Zirak, S. Chitosan-PVA-CNT Nanofibers as Electrically Conductive Scaffolds for Cardiovascular Tissue Engineering. Int. J. Biol. Macromol. 2019, 140, 278-287.
[25′] Chen, I.-H.; Wang, C.-C.; Chen, C.-Y Preparation of Carbon Nanotube (CNT) Composites by Polymer Functionalized CNT under Plasma Treatment. Plasma Processes Polym. 2010, 7, 59-63.
[26′] Wei, G.; Zhang, J.; Xie, L.; Jandt, K. D. Biomimetic Growth of Hydroxyapatite on Super Water-Soluble Carbon Nanotube-Protein Hybrid Nanofibers. Carbon 2011, 49, 2216-2226.
[27′] Prusty, R. K.; Rathore, D. K.; Ray, B. C. CNT/Polymer Interface in Polymeric Composites and Its Sensitivity Study at Different Environments. Adv. Colloid Interface Sci. 2017, 240, 77-106.
[28′] Khan, M. U.; Reddy, K. R.; Snguanwongchai, T.; Haque, E.; Gomes, V. G. Polymer Brush Synthesis on Surface Modified Carbon Nanotubes via in Situ Emulsion Polymerization. Colloid Polym. Sci. 2016, 294, 1599-1610.
[29′] Spitalsky, Z.; Tasis, D.; Papagelis, K.; Galiotis, C. Carbon Nanotube-Polymer Composites: Chemistry, Processing, Mechanical and Electrical Properties. Prog. Polym. Sci. 2010, 35, 357-401.
[30′] Homenick, C. M.; Lawson, G.; Adronov, A. Polymer Grafting of Carbon Nanotubes Using Living Free-Radical Polymerization. Polym. Rev. 2007, 47, 265-290.
[31′] Simmons, T. J.; Bult, J.; Hashim, D. P.; Linhardt, R. J.; Ajayan, P. M. Noncovalent Functionalization as an Alternative to Oxidative Acid Treatment of Single Wall Carbon Nanotubes with Applications for Polymer Composites. ACS Nano 2009, 3, 865-870.
[32′] Yu, H.; Jin, Y; Peng, F.; Wang, H.; Yang, J. Kinetically Controlled Side-Wall Functionalization of Carbon Nanotubes by Nitric Acid Oxidation. J. Phys. Chem. C 2008, 112, 6758-6763.
[33′] Fan, D.-Q.; He, J.-P.; Tang, W.; Xu, J.-T.; Yang, Y-L. Synthesis of Polymer Grafted Carbon Nanotubes by Nitroxide Mediated Radical Polymerization in the Presence of Spin-Labeled Carbon Nanotubes. Eur. Polym. J. 2007, 43, 26-34.
[34′] Choi, I. H.; Park, M.; Lee, S.-S.; Hong, S. C. Pyrene-Containing Polystyrene Segmented Copolymer from Nitroxide Mediated Polymerization and Its Application for the Noncovalent Functionalization of as-Prepared Multiwalled Carbon Nanotubes. Eur. Polym. J. 2008, 44, 3087-3095.
[35′] Wang, X.; Min, M.; Liu, Z.; Yang, Y; Zhou, Z.; Zhu, M.; Chen, Y; Hsiao, B. S. Poly(Ethyleneimine) Nanofibrous Affinity Membrane Fabricated via One Step Wet-Electrospinning from Poly(Vinyl Alcohol)-Doped PolyfEthyleneimine) Solution System and Its Application. J. Membr. Sci. 2011, 379, 191-199.
[36′] Farzamfar, S.; Naseri-Nosar, M.; Vaez, A.; Esmaeilpour, F.; Ehterami, A.; Sahrapeyma, H.; Samadian, H.; Hamidieh, A.-A.; Ghorbani, S.; Goodarzi, A.; Azimi, A.; Salehi, M. Neural Tissue Regeneration by a Gabapentin-Loaded Cellulose Acetate/Gelatin Wet-Electrospun Scaffold. Cellulose 2018, 25, 1229-1238.
[37′] Zheng, Y; Miao, J.; Maeda, N.; Frey, D.; Linhardt, R. J.; Simmons, T. J. Uniform Nanoparticle Coating of Cellulose Fibers during Wet Electrospinning. J. Mater. Chem. A 2014, 2, 15029-15034.
[38′] Shaker, A.; Hassanin, A. H.; Shaalan, N. M.; Hassan, M. A.; El-Moneim, A. A. Micropatterned Flexible Strain Gauge Sensor Based on Wet Electrospun Polyurethane/PEDOT: PSS Nanofibers. Smart Mater. Struct. 2019, 28, 075029.
[39′] Smit, E.; Büttner, U.; Sanderson, R. D. Continuous Yarns from Electrospun Fibers. Polymer (Guildf). 2005, 46, 2419-2423.
[40′] Wang, H.; Kong, L.; Ziegler, G. R. Aligned Wet-Electrospun Starch Fiber Mats. Food Hydrocolloids 2019, 90, 113-117.
[41′] Khil, M.-S.; Bhattarai, S. R.; Kim, H.-Y; Kim, S.-Z.; Lee, K.-H. Novel Fabricated Matrix via Electrospinning for Tissue Engineering. J. Biomed. Mater. Res., Part B 2005, 72, 117-124.
[42′] Wang, L.; Wu, Y; Guo, B.; Ma, P. X. Nanofiber Yarn/Hydrogel Core-Shell Scaffolds Mimicking Native Skeletal Muscle Tissue for Guiding 3D Myoblast Alignment, Elongation, and Differentiation. ACS Nano 2015, 9, 9167-9179.
[43′] Abedalwafa, M.; Wang, F.; Wang, L.; Li, C. Biodegradable Poly-Epsilon-Caprolactone (PCL) for Tissue Engineering Applications: A Review. Rev. Adv. Mater. Sci. 2012, 34, 123-140.
[44′] Ren, K.; Wang, Y; Sun, T.; Yue, W.; Zhang, H. Electrospun PCL/Gelatin Composite Nanofiber Structures for Effective Guided Bone Regeneration Membranes. Mater. Sci. Eng. C 2017, 78, 324-332.
[45′] Hwang, P. T. J.; Murdock, K.; Alexander, G. C.; Salaam, A. D.; Ng, J. I.; Lim, D.-J.; Dean, D.; Jun, H.-W. Poly(F-Caprolactone)/Gelatin Composite Electrospun Scaffolds with Porous Crater-like Structures for Tissue Engineering. J. Biomed. Mater. Res., Part A 2016, 104, 1017-1029.
[46′] Coimbra, P.; Santos, P.; Alves, P.; Miguel, S. P.; Carvalho, M. P.; de Sa, K. D.; Correia, I. J.; Ferreira, P. Coaxial Electrospun PCL/Gelatin-MA Fibers as Scaffolds for Vascular Tissue Engineering. Colloids Surf, B 2017, 159, 7-15.
[47′] Akbari, M.; Tamayol, A.; Bagherifard, S.; Serex, L.; Mostafalu, P.; Faramarzi, N.; Mohammadi, M. H.; Khademhosseini, A. Textile Technologies and Tissue Engineering: A Path Toward Organ Weaving. Adv. Healthcare Mater. 2016, 5, 751-766.
[48′] Pedde, R. D.; Mirani, B.; Navaei, A.; Styan, T.; Wong, S.; Mehrali, M.; Thakur, A.; Mohtaram, N. K.; Bayati, A.; Dolatshahi-Pirouz, A.; Nikkhah, M.; Willerth, S. M.; Akbari, M. Emerging Biofabrication Strategies for Engineering Complex Tissue Constructs. Adv. Mater. 2017, 29, 1606061.
[49′] Gupta, B.; Geeta; Ray, A. R. Preparation of Poly(F-Caprolactone)/Poly(R-Caprolactone-Co-Lactide) (PCL/PLCL) Blend Filament by Melt Spinning. J. Appl. Polym. Sci. 2012, 123, 1944-1950.
[50′] ASTM D3039/D3039M-17: Standard Test Method for Tensile Properties of Polymer Matrix Composite Materials. Annual Book of ASTM Standards. ASTM International, 2017, pp 1-13.
[51′] Zhang, M.; Zhao, M.; Jian, M.; Wang, C.; Yu, A.; Yin, Z.; Liang, X.; Wang, H.; Xia, K.; Liang, X.; Zhai, J.; Zhang, Y Printable Smart Pattern for Multifunctional Energy-Management E-Textile. Matter 2019, 1, 168-179.
[52′] Yin, Z.; Jian, M.; Wang, C.; Xia, K.; Liu, Z.; Wang, Q.; Zhang, M.; Wang, H.; Liang, X.; Liang, X.; Long, Y; Yu, X.; Zhang, Y Splash-Resistant and Light-Weight Silk-Sheathed Wires for Textile Electronics. Nano Lett. 2018, 18, 7085-7091.
[53′] Li, H.; Yan, S. Surface-Induced Polymer Crystallization and the Resultant Structures and Morphologies. Macromolecules 2011, 44, 417-428.
[54′] Walczak, Z. K. Processes of Fiber Formation; Elsevier Science, 2002.
[55′] Li, L.; Li, B.; Hood, M. A.; Li, C. Y Carbon Nanotube Induced Polymer Crystallization: The Formation of Nanohybrid Shish-Kebabs. Polymer 2009, 50, 953-965.
[56′] Singh, N. K.; Singh, S. K.; Dash, D.; Gonugunta, P.; Misra, M.; Maiti, P. CNT Induced β-Phase in Polylactide: Unique Crystallization, Biodegradation, and Biocompatibility. J. Phys. Chem. C 2013, 117, 10163-10174.
[57′] Urquijo, J.; Aranburu, N.; Dagréou, S.; Guerrica-Echevarria, G.; Eguiazábal, J. I. CNT-Induced Morphology and Its Effect on Properties in PLA/PBAT-Based Nanocomposites. Eur. Polym. J. 2017, 93, 545-555.
[58′] Vatankhah, E.; Semnani, D.; Prabhakaran, M. P.; Tadayon, M.; Razavi, S.; Ramakrishna, S. Artificial Neural Network for Modeling the Elastic Modulus of Electrospun Polycaprolactone/Gelatin Scaffolds. Acta Biomater. 2014, 10, 709-721.
[59′] Liu, S.; Luo, G.; Wei, F. Poly(p-Phenylene Terephthalamide)/Carbon Nanotube Composite Membrane: Preparation via Polyanion Solution Method and Mechanical Property Enhancement. Compos. Sci. Technol. 2015, 118, 135-140.
[60′] Jyoti, J.; Basu, S.; Singh, B. P.; Dhakate, S. R. Superior Mechanical and Electrical Properties of Multiwall Carbon Nanotube Reinforced Acrylonitrile Butadiene Styrene High Performance Composites. Composites, Part B 2015, 83, 58-65.
[61′] Zhang, P.; Qiu, D.; Chen, H.; Sun, J.; Wang, J.; Qin, C.; Dai, L. Preparation of MWCNTs Grafted with Polyvinyl Alcohol through Friedel-Crafts Alkylation and Their Composite Fibers with Enhanced Mechanical Properties. J. Mater. Chem. A 2015, 3, 1442-1449.
[62′] Coleman, J. N.; Khan, U.; Blau, W. J.; Gun'ko, Y K. Small but Strong: A Review of the Mechanical Properties of Carbon Nanotube-Polymer Composites. Carbon 2006, 44, 1624-1652.
[63′] Diez-Pascual, A. M.; Ashrafi, B.; Naffakh, M.; González-Dominguez, J. M.; Johnston, A.; Simard, B.; Martinez, M. T.; Gómez-Fatou, M. A. Influence of Carbon Nanotubes on the Thermal, Electrical and Mechanical Properties of Poly(Ether Ether Ketone)/Glass Fiber Laminates. Carbon 2011, 49, 2817-2833.
[64′] Firme, C. P.; Bandaru, P. R. Toxicity Issues in the Application of Carbon Nanotubes to Biological Systems. Nanomed. Nanotechnol. Biol. Med. 2010, 6, 245-256.
[65′] Wick, P.; Manser, P.; Limbach, L.; Dettlaffweglikowska, U.; Krumeich, F.; Roth, S.; Stark, W.; Bruinink, A. The Degree and Kind of Agglomeration Affect Carbon Nanotube Cytotoxicity. Toxicol. Lett. 2007, 168, 121-131.
[66′] Sun, H.; Zhou, J.; Huang, Z.; Qu, L.; Lin, N.; Liang, C.; Dai, R.; Tang, L.; Tian, F. Carbon Nanotube-Incorporated Collagen Hydro-gels Improve Cell Alignment and the Performance of Cardiac Constructs. Int. J. Nanomed. 2017, 12, 3109-3120.
[67′] Chiu, J.-J.; Chen, L.-J.; Chen, C.-N.; Lee, P.-L.; Lee, C.-I. A Model for Studying the Effect of Shear Stress on Interactions between Vascular Endothelial Cells and Smooth Muscle Cells. J. Biomech. 2004, 37, 531-539.
[68′] Vunjak-Novakovic, G.; Altman, G.; Horan, R.; Kaplan, D. L. Tissue Engineering of Ligaments. Annu. Rev. Biomed. Eng. 2004, 6, 131-156.
[69′] Aubin, H.; Nichol, J. W.; Hutson, C. B.; Bae, H.; Sieminski, A. L.; Cropek, D. M.; Akhyari, P.; Khademhosseini, A. Directed 3D Cell Alignment and Elongation in Microengineered Hydrogels. Biomaterials 2010, 31, 6941-6951.
[70′] Ranjan, A.; Webster, T. J. Increased Endothelial Cell Adhesion and Elongation on Micron-Patterned Nano-Rough Poly-(Dimethylsiloxane) Films. Nanotechnology 2009, 20, 305102.
[71′] Wang, C.; Baker, B. M.; Chen, C. S.; Schwartz, M. A. Endothelial Cell Sensing of Flow Direction. Arterioscler. Thromb. Vasc. Biol. 2013, 33, 2130-2136.
[72′] Tandon, N.; Goh, B.; Marsano, A.; Chao, P. H. G.; Montouri-Sorrentino, C.; Gimble, J.; Vunjak-Novakovic, G. Alignment and Elongation of Human Adipose-Derived Stem Cells in Response to Direct-Current Electrical Stimulation. In Proceedings of the 31st Annual International Conference of the IEEE Engineering in Medicine and Biology Society: Engineering the Future of Biomedicine; EMBC 2009, 2009; pp 6517-6521.
[73′] Iba, T.; Sumpio, B. E. Morphological Response of Human Endothelial Cells Subjected to Cyclic Strain in Vitro. Microvasc. Res. 1991, 42, 245-254.
[74′] Wu, Y; Wang, L.; Guo, B.; Ma, P. X. Interwoven Aligned Conductive Nanofiber Yarn/Hydrogel Composite Scaffolds for Engineered 3D Cardiac Anisotropy. ACS Nano 2017, 11, 5646-5659.
[75′] Hamedani, B. A.; Navidbakhsh, M.; Tafti, H. A. Comparison between Mechanical Properties of Human Saphenous Vein and Umbilical Vein. Biomed. Eng. Online 2012, 11, 59.
[76′] McKenna, K. A.; Hinds, M. T.; Sarao, R. C.; Wu, P.-C.; Maslen, C. L.; Glanville, R. W.; Babcock, D.; Gregory, K. W. Mechanical Property Characterization of Electrospun Recombinant Human Tropoelastin for Vascular Graft Biomaterials. Acta Biomater. 2012, 8, 225-233.
[77′] Claes, E.; Atienza, J. M.; Guinea, G. V.; Rojo, F. J.; Bernal, J. M.; Revuelta, J. M.; Elices, M. Mechanical Properties of Human Coronary Arteries. In 2010 Annual International Conference of the IEEE Engineering in Medicine and Biology Society; EMBC 2010, 2010; pp 3792-3795.
[78′] Motte, S.; Kaufman, L. J. Strain Stiffening in Collagen i Networks. Biopolymers 2013, 99, 35-46.
[79′] Wang, J. H.-C.; Guo, Q.; Li, B. Tendon Biomechanics and Mechanobiology—A Minireview of Basic Concepts and Recent Advancements. J. Hand Ther. 2012, 25, 133-141.
[80′] Jaspers, M.; Dennison, M.; Mabesoone, M. F. J.; MacKintosh, F. C.; Rowan, A. E.; Kouwer, P. H. J. Ultra-Responsive Soft Matter from Strain-Stiffening Hydrogels. Nat. Commun. 2014, 5, 5808.
[81′] Freeman, J. W.; Woods, M. D.; Laurencin, C. T. Tissue Engineering of the Anterior Cruciate Ligament Using a Braid-Twist Scaffold Design. J. Biomech. 2007, 40, 2029-2036.
[82′] Maziz, A.; Concas, A.; Khaldi, A.; Stålhand, J.; Persson, N.-K.; Jager, E. W. H. Knitting and Weaving Artificial Muscles. Sci. Adv. 2017, 3, No. e1600327.
[83′] Mironov, V.; Visconti, R. P.; Kasyanov, V.; Forgacs, G.; Drake, C. J.; Markwald, R. R. Organ Printing: Tissue Spheroids as Building Blocks. Biomaterials 2009, 30, 2164-2174.
[84′] Taki, A.; Kermani, A.; Ranjbarnavazi, S. M.; Pourmodheji, A. Overview of Different Medical Imaging Techniques for the Identification of Coronary Atherosclerotic Plaques. In Computing and Visualization for Intravascular Imaging and Computer-Assisted Stenting; Elsevier Science, 2017; pp 79-106.
[85′] Dong, H.-M.; Ichimura, K.; Sakai, T. Structural Organization of Hepatic Portal Vein in Rat with Special Reference to Musculature, Intimal Folds, and Endothelial Cell Alignment. Anat. Rec. 2010, 293, 1887-1895.
[86′] Kroon, J.; Heemskerk, N.; Kalsbeek, M. J. T.; De Waard, V.; Van Rijssel, J.; Van Buul, J. D. Flow-Induced Endothelial Cell Alignment Requires the RhoGEF Trio as a Scaffold Protein to Polarize Active Rac1 Distribution. Mol. Biol. Cell 2017, 28, 1745-53.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method of making a scaffold for tissue engineering comprising:
providing a textile layer formed of a plurality of yarns, wherein the plurality of yarns are formed of interlocking bundles of fibers formed from a first polymer or a first polymer mixture; and
forming one or more substrate layers of a second polymer or a second polymer mixture onto the textile layer having a pre-defined thickness.

2. The method of claim 1, wherein the textile layer is formed of the plurality of yarns by a weaving operation, a knitting operation, a crocheting operation, a knotting operation, a tatting operation, a felting operation, a bonding operation, or a braiding operation to have a pre-defined textile pattern for cell growth of a population of cells onto the scaffold.

3. The method of claim 1, further comprising:
forming a yarn of the plurality of fibers by electrospinning the first polymer or the first polymer mixture into the plurality of fibers;
drawing the plurality of fibers out of an electrospinning bath to form the bundles; and
forming the yarn from the bundles.

4. The method of claim 1, wherein the one or more substrate layers is an electrospun mat.

5. The method of claim 3, wherein electrospinning of the fiber comprises wet electrospinning.

6. The method of claim 3, wherein the step of drawing the plurality of fibers out of the electrospinning bath to form the bundles further comprises winding the plurality of drawn fibers around a roller.

7. The method of claim 3, further comprising varying a drawing speed to generate (i) a desired fiber alignment of the plurality of fibers, (ii) a desired bundle diameter of the plurality of fibers, or a combination thereof.

8. The method of claim 3, wherein the first polymer or the first polymer mixture comprises polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, alginate, chitosan, agarose, fibrin, hyaluronic acid, a polyethylene glycol (PEG), elastin, silk fibroin, copolymers thereof, and blends thereof.

9. The method of claim 4, wherein the second polymer or the second polymer mixture comprises polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, alginate, chitosan, agarose, fibrin, hyaluronic acid, a polyethylene glycol (PEG), elastin, silk fibroin, or copolymers thereof, and blends thereof.

10. The method of claim 1, further comprising seeding a population of cells onto the scaffold.

11. The method of claim 1, wherein forming the textile layer comprises crocheting the plurality of yarns with a pre-defined crochet hook size to provide the scaffold with a pre-defined mechanical property selected from the group consisting of a pre-defined resilience or range, a pre-defined elastic modulus or range, a pre-defined maximum strain or range, and a pre-defined maximum stress or range, or any combination thereof.

12. The method of claim 2, further comprising:
electrospinning the one or more substrate layers; and
varying the duration of the electrospinning step to achieve a desired substrate layer thickness.

13. The method of claim 3, wherein the electrospinning bath comprises a liquid having a concentration in the electrospinning bath to form the plurality of fibers having a pre-defined mechanical property with the one or more substrate layers, wherein the pre-defined mechanical property is selected from the group consisting of a pre-defined resilience or range, a pre-defined elastic modulus or range, a pre-defined maximum strain or range, and a pre-defined maximum stress or range, or any combination thereof.

14-26. (canceled)

27. A scaffold for tissue engineering formed by the method of claim 1, the scaffold comprising:
a textile layer formed of a plurality of yarns, wherein the plurality of yarns are formed of interlocking bundles of fibers formed from a first polymer or a first polymer mixture; and
one or more substrate layers comprising a second polymer or a second polymer mixture formed onto or attached to the textile layer.

28-34. (canceled)

35. A therapeutic method comprising:
providing the scaffold of claim 27; and
implanting the scaffold into or onto a subject.

36. A method of fabricating composite yarns, the method comprising:
forming fibers of one or more polymers in a carbon nanomaterial bath, the carbon nanomaterial bath can include a carbon nanomaterial suspended in a liquid;
coating the fibers with the carbon nanomaterial to form fibers;
extracting the fibers from the carbon nanomaterial bath; and
interlocking bundles of fibers to form composite yarns, the composite yarns can include the one or more polymers and carbon nanomaterial.

37-43. (canceled)

44. A method of fabricating a scaffold for tissue engineering, the method comprising:
fabricating composite yarns according to the method of claim 36; and
forming a scaffold comprising the composite yarns.

45-50. (canceled)

51. A composite yarn comprising:
a yarn core comprising one or more polymers, and
carbon nanomaterial on the surface of the yarn core,
wherein the yarn core is used to form a textile layer of a scaffold for tissue engineering, wherein the yarns are formed of interlocking bundles of fibers comprising the one or more polymers and the carbon nanomaterial.

52-57. (canceled)

58. A tissue engineering scaffold comprising the composite yarns of claim 51.

59. A method of promoting cell adhesion to a tissue engineered scaffold, the method comprising:
fabricating composite yarns according to the method of claim 36;
fabricating a tissue engineered scaffold according to the method of claim 44; and
contacting the tissue engineered scaffold with cells in an environment that promotes cell viability.

60-64. (canceled)
